(19) United States
(12) Patent Application Publication
(10) Pub. No.: US 2024/0410020 A1
(43) Pub. Date: Dec. 12, 2024

(54) CANCER DRIVER MUTATION DIAGNOSTICS

(71) Applicant: Asaf HELLMAN, Kibbutz Yotvata (IL)

(72) Inventor: Asaf HELLMAN, Kibbutz Yotvata (IL)

(21) Appl. No.: 18/726,588

(22) PCT Filed: Jan. 3, 2022
(86) PCT No.: PCT/IL2022/050008

§ 371 Date: Jul. 3, 2024

(51) Int. Cl. C12Q 1/6886; G16B 40/00

(52) U.S. Cl. CPC C12Q 1/6886; G16B 40/00; C12Q 2600/154; C12Q 2600/156; C12Q 2600/158

(57) ABSTRACT

Methods for determining a driver gene of a pathological condition are provided. Kits and computer program products for doing same are also provided.

### CROSS-REFERENCE TO RELATED APPLICATIONS

This application claims the benefit of priority of U.S. Provisional Patent Application No. 63/133,393 filed Jan. 3, 2021, the contents of which are incorporated herein by reference in their entirety.

### FIELD OF INVENTION

The present invention is in the field of cancer diagnostics.

### BACKGROUND OF THE INVENTION

While malfunction of five to eight cancer-initiating (driver) genes is assumed to stand at the root of all cancers, alterations of protein-coding sequences have not been accountable for most common malignancies, including human glioblastoma multiforme (GBM). Non-coding regulatory mutations have been suggested to drive these “dark matter” tumors, but limited resolution of available cis-regulatory maps has hindered full examination of this theory. Shadowing and redundancy, frequently observed among cis-residing regulatory elements, further confound detection of causative mutation events. Hence, mapping of cis-regulatory circuits of cancer genes and clarifying their structures, components and interactions, are key to understanding cancer development.

Transcriptional silencers, also referred to as negative-or anti-enhancers, are DNA sequences that, upon binding of repressors or co-repressors, reduce transcription potential of interacting gene promoters. Silencers are well documented in model genomes, as well as in humans. Silencers and enhancers co-exist in mouse and human cancer gene regions and may interact over short or long (tens to millions of base pairs) distances to co-regulate gene expression. Thorough analyses of silencers and their interactions with enhancers in relation to cancer gene regulation have not yet been reported.

Among chromatin markers, DNA methylation is unique as a quantitative and sensitive indicator of regulatory activity. It also distinctively discriminates activity levels at site-specific resolution. Methylation of gene promoters often limits accessibility to transcriptional activators, denoting a negative effect on expression. Among non-promoter regulatory sites, however, positive and negative associations of methylation with gene expression are mutually common and may reflect various regulatory mechanisms. One of the mechanisms underlying positive associations is methylation-mediated silencing of repressor genes, which promotes expression of controlled genes. Such secondary effects may be efficiently detected by analyzing inter-genic expression interactions. Another mechanism is coupling of methylation with transcription, which is particularly notable along the transcribed regions of genes (gene bodies). Alternatively, positive correlations that are not due to secondary effects or to the gene body methylation pattern, might reflect primary regulatory activities, e.g., methylation-driven binding of activators to enhancers, or elimination of repressors from silencers. An abundance of methyl-attracting and methyl-avoiding activators and repressors has been described in the human genome, allowing a range of such scenarios. Evidence for direct effects of DNA methylation on transcriptional enhancers have been presented, but the effect on silencers remains unknown.

The spectrum of possible interactions between enhancers, silencers and various methyl-attracting and methyl-avoiding activators and repressors, hinders the elucidation of gene regulatory circuits. There is a great need to resolve this complexity and uncover gene cis-regulatory structures and the rules governing their normal and malignant activities. Such a discovery will help map driver mutations that are outside of the coding region of genes and open new avenues for treatment of these heretofore poorly defined malignancies.

### SUMMARY OF THE INVENTION

The present invention provides methods for determining a driver gene of a pathological condition by measuring DNA methylation of non-promoter cis-regulatory elements of potential driver genes and selecting at least one gene whose cis-regulatory methylation produces an abhorrent regulatory effect.

According to a first aspect, there is provided a method for determining a driver gene of a pathological condition in a subject in need thereof, the method comprising:
a. receiving measurements of DNA methylation within a plurality of non-promoter cis-regulatory sequences from the subject;
b. determining from the received measurements a total regulatory effect of non-promoter cis-regulatory sequences upon at least one potential driver gene of the pathological condition; and
c. selecting the at least one potential driver gene as a driver of the pathological condition in the subject when the total regulatory effect is beyond a predetermined threshold;
thereby determining a driver of a pathological condition in a subject.

According to another aspect, there is provided a kit, comprising nucleotide probes that hybridize to non-promoter cis-regulatory sequences of a plurality of genes selected from genes provided in Table 3, Table 4 or Table 6.

According to another aspect, there is provided a computer program product for determining a driver gene for a pathological condition, comprising a non-transitory computer-readable storage medium having program code embodied thereon, the program code executable by at least one hardware processor to:
a. receive measurements of DNA methylation within a plurality of non-promoter cis-regulatory sequences;
b. determine from the received measurements a total regulatory effect of non-promoter cis-regulatory sequences upon at least one potential driver gene of the pathological condition; and
c. select the at least one potential driver gene as a driver of the pathological condition when the total regulatory effect is beyond a predetermined threshold.

According to some embodiments, the measurements of DNA methylation are obtained by:
a. obtaining DNA from a biological sample from the subject;
b. isolating a plurality of cis-regulatory sequences from the obtained DNA; and
c. measuring DNA methylation within the plurality of isolated cis-regulatory sequences.

According to some embodiments, the measuring DNA methylation comprises bisulfite sequencing of the plurality of isolated sequences.

According to some embodiments, the DNA is selected from genomic DNA (gDNA), mitochondrial DNA (mtDNA), cell-free DNA (cfDNA) and cell-free fetal DNA (cffDNA).

According to some embodiments, the biological sample is selected from: tissue, blood, lymph, cerebral spinal fluid, urine, breast milk, feces, saliva, tumor tissue and tumor fluid.

According to some embodiments, the tissue is a tumor biopsy.

According to some embodiments, the isolating comprises binding probes to the cis-regulatory sequences and isolating the hybridized probes.

According to some embodiments, the probe binds histone 3 lysine 4 monomethylated (H3K4me1) chromatin.

According to some embodiments, the probe is a nucleic acid probe that hybridizes to the cis-regulatory sequence.

According to some embodiments, the probe comprises a non-nucleic acid capture moiety and wherein the isolating comprises capturing the capture moiety to a capturing molecule.

According to some embodiments, the plurality of non-promoter cis-regulatory sequences are located within 1 megabase upstream or downstream of a transcriptional start site of the at least one potential driver gene.

According to some embodiments, the plurality of non-promoter cis-regulatory sequences are selected from enhancer and repressor elements.

According to some embodiments, the plurality of non-promoter cis-regulatory sequences comprises at least one repressor element.

According to some embodiments, the plurality of non-promoter cis-regulatory sequences comprises at least 4 distinct cis-regulatory sequences.

According to some embodiments, the regulatory effect of each cis-regulatory sequence is determined independently or is determined in combination with at least one other cis-regulatory sequence.

According to some embodiments, at least one measured cis-regulatory sequence comprises more than one CpG dinucleotide and wherein a measurement from at least one of the more than one CpG dinucleotides within the cis-regulatory sequence is received.

According to some embodiments, the determining comprises at least one of:
a. testing each of the plurality of non-promoter cis-regulatory sequences in an expression assay, wherein the assay measures the regulatory effect of a non-promoter cis-regulatory sequence on expression of a coding sequence and wherein the testing comprises testing methylated and unmethylated copies of each of the plurality of non-promoter cis-regulatory sequences;
b. comparing the received measurements to a database comprising potential driver genes, methylation status of non-promoter cis-regulatory sequences of the database genes, and regulatory effects of the non-promoter cis regulatory sequences on the database genes; and
c. applying a machine learning algorithm to the received measurements, wherein the machine learning algorithm has been trained on non-promoter cis-regulatory sequences with known methylation status and known regulatory effect on a driver gene.

According to some embodiments, a regulatory effect of each non-promoter cis-regulatory sequence is determined separately and summed to produce the total regulatory effect, or wherein total regulatory effect for at least two non-promoter cis-regulatory sequences is determined simultaneously.

According to some embodiments, the machine learning algorithm has been trained on:
a. single non-promoter cis-regulatory sequences;
b. genes and at least one of each gene's non-promoter cis-regulatory sequences;
c. genes and a plurality of each gene's non-promoter cis-regulatory sequences; or
d. genes and all of each gene's non-promoter cis-regulatory sequences.

According to some embodiments, the predetermined threshold is derived from a predetermined standard regulatory effect for the non-promoter cis-regulatory sequences of the at least one potential driver gene, and wherein the predetermined standard regulatory effect is determined in any one of:
a. cells grown in culture;
b. cells from a healthy subject; and
c. cells from a subject suffering from a pathological condition.

According to some embodiments, measurements of DNA methylation within non-promoter cis-regulatory sequences of a panel of potential driver genes are received.

According to some embodiments, the method further comprises confirming aberrant expression of the selected driver gene in a sample from the subject.

According to some embodiments, the pathological condition is cancer.

According to some embodiments, the cancer is glioblastoma.

According to some embodiments, a potential driver gene is any one of the driver genes provided in Table 3 or any of the genes provided in Table 6.

According to some embodiments, total regulatory effect on a panel of driver genes is determined, and the panel is selected from the genes provided in Table 6.

According to some embodiments, the non-promoter cis-regulatory sequences are selected from sequences located between genomic positions provided in Table 4.

According to some embodiments, the method of the invention is for diagnosing a pathological condition or increased risk of developing a pathological condition.

According to some embodiments, the method further comprises administering a medicament that targets the driver, DNA methylation, or DNA methylation machinery.

According to some embodiments, the plurality of genes is selected from the genes provided in Table 6.

According to some embodiments, the non-promoter cis-regulatory sequences are located between genomic positions provided in Table 4.

According to some embodiments, the kit of the invention is for diagnosing and/or prognosing a pathological condition.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D: Methylation-centered interrogation of functional gene-associated regulatory networks. (1A) Cartoon showing regulatory chromatin blocks were identified among glioblastoma (GBM) tumors in 2-Mb regions surrounding 125 driver and 52 reference cancer genes. H3K4Mel-marked/H3K27ac-variable chromatin segments encompassing methylation and sequence variations were captured from GBM tumor biopsies using biotinylated RNA probes. (1B-C) The obtained target-enriched libraries, representing the spectrum of GBM regulatory variation, were used for functional annotation of the targeted regions (1B) before or after DNA methylation (1C), or subjected to deep bisulfite sequencing providing methylation-site resolution of gene-associated positive and negative regulatory circuits. (1D) The integration of functional and gene-associated data allows disclosing of cis-regulatory structures.

FIGS. 2A-G. DNA methylation modifies the transcriptional effect of enhancers and silencers. (2A) Method: Putative regulatory DNA segments were captured from GBM tumors and allowed to drive self-transcription in T98G GBM cells, following complete de-methylation or after in-vitro methylation of the expression vector. Local DNA to RNA ratios, relative to the total DNA to RNA ratio, denotes transcriptional activity score (TAS) of the evaluated DNA segments. (2B) Maps of example genomic regions containing enhancers and silencers. Local activity scores are shown as bars which are positive for enhancers and negative for silencers. H3K27ac bars denote the fraction of the analyzed GBM tumors which displayed this marker of active regulatory chromatin. Bound TFs in a variety of different cell types are given as a reference for the general regulatory activity of the regions (2C) Pie chart of frequencies of regulatory elements that were annotated as functional silencers or enhancers along the targeted gene domains. (2D) Bar charts of regulatory chromatin characteristics of enhancer and silencer loci. Level of transcription factors binding (TFB), factor variety (breadth), and DNase I hyper-sensitivity are shown across a variety of different cell types (ENCOD data). (2E) Effect of DNA methylation on 20-quantiles groups of regulatory elements. Average activity levels of the groups before and after methylation (TAS, Methyl.TAS), as well as the average shift in activity upon methylation (ATAS), are shown. (2F) Pie chart of methylation effects on silencers and enhancers. (2G) Graph of the effect of DNA methylation on TAS level of the regulatory groups shown in panel 2F. The arrow heads indicate TAS level post-methylation. Fractions of sites that switched activities are given below. ** p<1E-20.

FIGS. 3A-E. Methylation-based deciphering of cis-regulatory networks in bona fide tumor chromatin. (3A) Methylation-based association of regulatory sites with controlled genes. (3B) Top: Examples of functional enhancer and silencer elements that were identified along the SMO driver gene domain through the massively parallel assay presented in FIG. 2A-B. Even-sized windows (about ×20 larger than the median size of regulatory units) are shown. Bottom: Correlations between DNA methylation and SMO expression levels across GBM tumors, for representative methylation sites in the functional elements. (3C) Bar charts showing validation of the predicted effects of SMO regulatory units via manipulations of GBM genomes. Left: Effects of deletions the enhancer labelled ‘A’ in FIG. 3B, or of the silencer ‘D’, versus mock genomic targeting by scrambled targeting guides. Right: Effect of enhancer deletion on the background of a silencer deletion. Bars represent standard deviations based on ≥ four biological replications. (3D) Gene-associated sites reveal networks of homogenous, positive or negative regulatory units that cooperatively control SMO expression variation. (3E) A heatmap diagram showing the correlation between the methylation level of each methylation sites in the SMO domain, and the methylation levels of all other sites in the domain, across 24 GBM tumors. In the tumors with the highest expression of the gene, enhancers were unmethylated and silencer were methylated, and vice versa. * p<0.05; ** p<0.005

FIGS. 4A-E. Networks of epigenetically-tuned transcriptional silencers and enhancers govern disease driver-gene malfunction. (4A) Development of methylation-based models of gene expression variation. (4B) Example models. Left: methylation versus expression of the sites consisting of the best prediction model of the TNFAIP3 gene. Right: predicted versus observed variation of the TNFAIP3 and the SMO genes across the tumors. SMO model was based on the four sites shown in FIG. 3B. (4C) Verified models of gene-expression variation. Models with up to 2-fold difference between predicted and observed expression levels in at least 20 of the 24 leave-one-out rounds considered success. Verified models of driver genes are presented. Verified models of reference genes are given in FIG. 15. (4D) Pie graph of participation of silencers and enhancers in confirmed cis-regulatory networks. (4E) Table of the numbers of driver-gene per tumors that are affected by sequence or methylation mutations in their coding or regulatory components. SNV: Single Nucleotide Variation; CNV: Copy Number Variation. Mis-regulated genes are genes that display >2-fold expression deviation from normal brain in the given tumor sample. Highlighted cell indicates tumors with at least five (orange) or eight (yellow) mutated driver-genes.

FIGS. 5A-B. Methylation-expression associations in various cancer types. (5A) Bar chart of percentages of negatively and positively-associated sites that carry H3K4me1 marks, out of all gene-associated sites across various types of cancer. The analysis was performed on public TCGA data. (5B) Bar chart of percentages of gene-associated methylation sites in given types of cancers, which displayed the opposite effects on expression of the associated genes in at least one other cancer type.

FIGS. 6A-B. Overlapping between targeted gene domains (+/−1 Mb of TSS) and Hi-C-based topological associated domains (TAD). (6A) Bar graph of fractions of genes without TADs following Hi-C analysis of three GBM samples (25 kb resolution), and fractions of gene-associated sites that related to genes without TADs, out of all uncovered gene-associated sites. (6B) Bar graph of fractions of genes with Hi-C-based TADs, for which the targeting criteria provide full coverage of the gene TAD, and fractions of gene-associated sites within Hi-C-based TADs, out of all uncovered gene-associated sites.

FIG. 7. Overall flow and terminology of the study. (1) Domains of the human genome that have been explored, including one million base pairs to each side of the transcription start sites (TSSs) of 177 driver and reference cancer genes. (2) Within these domains, the regions showing marking of regulatory chromatin are located across the analyzed tumors. (3) Biotinylated RNA Probes (120 bp each) were designed to cover all CpG methylation sites within the identified chromatin regions. (4) Randomly sheared DNA segments of tumor genomic DNAs were allowed to attach to (partially or fully) overlapping RNA probes. (5) Pulling-out the attached segments yielded a library of captured DNA segments of various sizes (mean=224 bp). The distribution of the sizes of the captured segments in an exemplary library (sample #100) is shown. (6) The captured segments were then integrated into gene-reporting vectors, forming a library of reporter assays. (7) Enhancer or silencer functionalities were analyzed in 500 bp (50% overlapping) windows across the studied regions, before or after methylation of the vectors, thus allowing location of significant (FDR q value <0.05) methylation-sensitive and insensitive enhancer & silencer elements and uncovering the general rules of enhancers' and silencers' responses to extreme methylation conditions (8). (9) In parallel, the libraries of captured DNA segments were sequenced with or without bisulfite treatment. (10) The correlation between the methylation levels of each methylation site and the expression of the explored genes over the tumors were analyzed, and the data was used to produce domain-wide correlation maps. (11) Finally, the general roles learned from the simplified experimental assay, together with the actual data collected from the tumors, were used to deduce the actual size of enhancer and silencer regulatory units (average size=834 bp, median=333 bp), and their participation in cis-regulatory networks.

FIGS. 8A-B. Functional annotation of isolated regulatory elements. (8A) Bar chart of the distribution of silencer and enhancer elements in the targeted gene domains. (8B) Chart of fractions of enhancers and silencers that bind activating, repressing, or both activating and repressing transcription factors across ENCODE cell lines. The list of activators includes: RNAP, GATA2, GATA3, EP300, BCL3, NFATC1, HNF4A, HNF4G, ELK4, ELK1 and IRF1. The repressors list includes: REST, YY1, ZBTB33, SUZ12, EZH2, RCOR1, CTCF, SMC3, RAD21, PAX5 and RUNX3.

FIGS. 9A-G. Characteristics of methylation-sensitive and methylation-insensitive elements. (9A) Assay: Genomic segments (mean size=224 bp) were captured from a GBM tumor, ligated downstream to minimal promoters and allowed to drive transcription in T98G glioblastoma (GBM) cells. Plasmid DNA and RNA were then extracted from the GBM cells and sequenced. The ratio between DNA and RNA copy numbers, normalized to total DNA and RNA levels, denotes the transcriptional activity of the targeted elements. Example enhancer and silencer elements are shown. DNA and RNA copy numbers are indicated to the left of each segment. (9B) The enhancer and silencer shown in 9A are shown following in-vitro DNA methylation. (9C) Pie chart of the fractions of methylation-sensitive and methylation-insensitive elements. (9D) Bar graph of transcription-factor binding (TFB) scores. (9E) Bar graph of transcription factor (TF) variety (breadth). (9F) Bar graph of DNase I hyper-sensitivity (HS). (9G) Bar graph of average number of CpG methylation sites per element (density). For reference, analyses in 500 bp, 50% overlapping windows across the genome are presented.

FIG. 10. Eliminated associations due to possible secondary effects. Prohibited association between 1) methylation of a promoter site and expression of a possible activator of the indicated gene A; 2) methylation of a promoter site and expression of a possible repressor of the indicated gene A; 3) methylation of a gene-body site and expression of a possible activator of the indicated gene A; and 4) methylation of a gene-body site and expression of a possible repressor of the indicated gene A.

FIG. 11. Alignment of positive and negative units with silencers and enhancers. A schematic map showing the five regulatory units of the SMO driver gene. Grey: negative methylation-expression associations. White: positive associations. Functional and methylation analyses of SMO enhancer and silencer units. Transcriptional Activity Score (TAS) analyzed through reporter assay analysis is shown, as is DNA methylation levels of the 24 analyzed GBM tumors. Chromatin marks and bound transcription factors are also shown. Genomic coordination of the knockout regions in the genomic editing experiments (Scc FIGS. 3C and 12A-C).

FIGS. 12A-C. Compliance between assays. (12A) Pie charts of fractions of functional elements located by the gene-reporting assay, adjacent (≤500 bp) to a GBM-related site. TAS was calculated in 500 bp (50% overlapping) windows. (12B) Pic charts of fractions of GBM-related sites adjacent to a functional element. TAS was calculated in 500 bp (50% overlapping) windows. (12C) Pie chart of the impact of DNA methylation on regulatory activity of GBM-related sites. The analysis performed as in FIG. 2F, but for 4,434 negatively-correlating sites with positive TAS (enhancers) and 3,274 positively-correlating sites with negative TAS (silencers). TAS was calculated for the DNA segments overlapping the given sites.

FIGS. 13A-B. Methylation-methylation coordination maps of genes with multiple regulatory circuits. (13A-B) Coordination between the methylation levels of (13A) SMO-associated sites and (13B) TNFAIP3-associated sites. Genomic locations of the associated sites are given to the left. Red label with rightward slope: positive methylation versus expression associations. Blue label with leftward slope: negative methylation versus expression associations. The sites producing best prediction models (see FIG. 4A-E) are highlighted. Each square in the matrixes show the methylation versus methylation correlation (R) between two of the associated sites. Genomic maps showing the locations of the associated sites (red and blue bars), of the associated genes (purple), and the site order in the matrix are shown above. Two representative genes are provided.

FIG. 14. Gene-specific networks. Matrix showing the coordination between the methylation levels of sites associated with the GDF15 (purple) or the IF130 (green) genes are shown. Each square in the matrixes show the methylation versus methylation correlation (R) between two of the associated sites. Blue label with leftward slope: negative methylation versus expression associations. Red label with rightward slope: positive associations. White squares denote no correlation (R2<0.1). A representative gene is shown.

FIG. 15. Log 2 of the differences between predicted and observed gene expression levels for reference (non-driver) genes with developed models. Box plots describe the distributions of prediction accuracy in 24 independent tests.

FIG. 16. Prediction qualities of gene-expression models developed by lasso-type analysis. Gene-expression models were developed and validated as described in FIG. 4C but using Lasso regression without limiting the number of participating methylation sites. The distribution of (log 2) predicted-versus-observed expression differences over 24 model-developing repeats using the leave-one-out method are presented for the genes shown in FIG. 4C.

FIG. 17. Cellular functions of mis-regulated driver genes for which a methylation-based model of expression variation was developed and verified.

### DETAILED DESCRIPTION OF THE INVENTION

The present invention, in some embodiments, provides methods for determining a driver gene of a pathological condition. The present invention further concerns kits and computer program products for performance of the methods of the invention.

The invention is based on the surprising finding that DNA methylation induces enhancers and silencers to acquire new activity setpoints within wide ranges of potential regulatory effects, varying between strong transcriptional enhancing to strong silencing. Extensive analysis of methylation-expression associations revealed the organization of domain-wide cis-regulatory networks and highlighted key regulatory sites which provide pivotal contributions to the network outputs. Consideration of these effects through mathematical models of gene expression variations identified prime molecular events underlying cancer-genes mis-regulation in hitherto unexplained tumors. Of the observed gene-malfunctioning events, gene mis-regulation due to epigenetic retuning of networked enhancers and silencers dominated driver-genes mutagenesis, compared with other types of mutation including coding and regulatory sequence alterations.

Silencers and enhancers are known to cooperate in the regulation of gene transcription, but without thorough understanding of the mechanism and the factors that guide the mode of action of regulatory sites and the cooperation between them, it had been impossible to characterize the effect on normal and abnormal gene activities. To deal with this challenge, a method for detection and annotation of the organization, activities and interactions of silencers and enhancers in cancer tumors was developed.

By a first aspect, there is provided a method for determining a driver gene of a condition in a subject in need thereof, the method comprising:
a. receiving measurements of DNA methylation within a plurality of cis-regulatory sequence from the subject;
b. determining from the received measurements a total regulatory effect of cis-regulatory sequences upon at least one potential driver gene of the pathological condition; and
c. selecting the at least one potential driver gene as a driver of the pathological condition in the subject when the total regulatory effect is beyond a predetermined threshold;
thereby determining a driver gene of a pathological condition in a subject.

In some embodiments, the subject is a mammal. In some embodiment, the subject is a human. In some embodiments, the subject suffers from the condition. In some embodiments, the condition is a pathological condition. In some embodiments, the subject suffers from cancer. In some embodiments, the pathological condition is cancer. In some embodiments, the condition is a pathological condition. In some embodiments, the condition is a condition driven by at least one gene. In some embodiments, the condition is a condition driven by a driver gene.

In some embodiments, the cancer is a neurological cancer. In some embodiments, the cancer is a brain cancer. In some embodiments, the cancer is glioblastoma. In some embodiments, the cancer is glioblastoma multiforme. In some embodiments, the cancer is driven by a driver gene. In some embodiments, the cancer is driven by at least one driver gene. In some embodiments, the cancer is selected from breast cancer, lung cancer, uterine cancer, head and neck cancer, colon cancer, rectal cancer, bladder cancer, urothelial cancer, kidney cancer, renal cancer, ovarian cancer, and leukemia. In some embodiments, the cancer is selected from an adenocarcinoma, carcinoma, endometrial carcinoma, blastoma, glioblastoma, squamous cell carcinoma, clear cell carcinoma, and serous carcinoma. In some embodiments, the cancer is selected from breast adenocarcinoma, lung adenocarcinoma, lung squamous cell carcinoma, uterine corpus endometrial carcinoma, glioblastoma multiforme, head and neck squamous cell carcinoma, colon and rectal carcinoma, bladder urothelial carcinoma, kidney renal clear cell carcinoma, ovarian serous carcinoma, and acute myeloid leukemia.

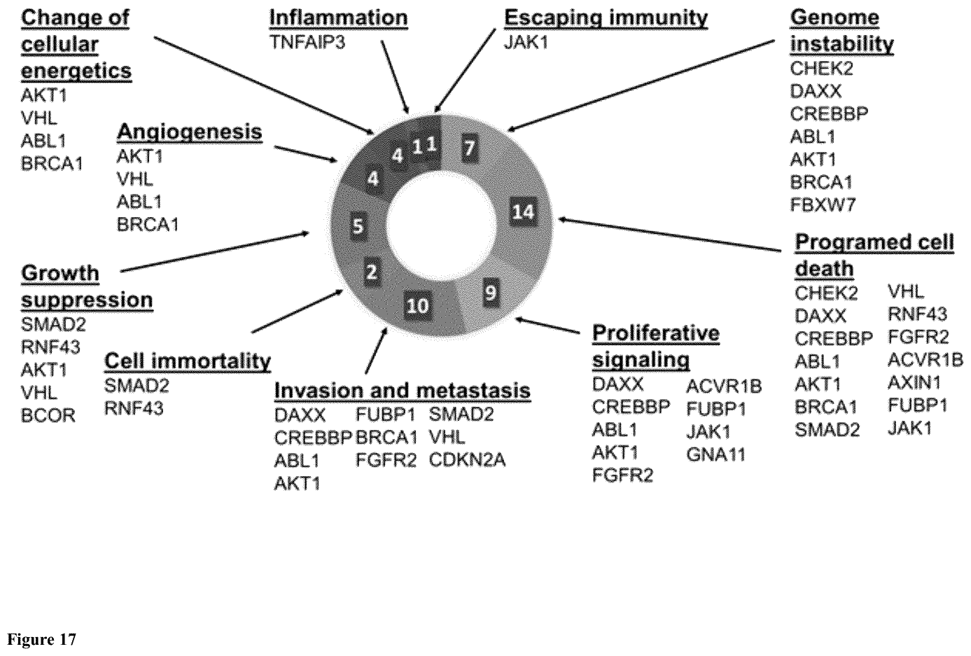

In some embodiments, a driver gene is a gene whose misexpression causes the condition. In some embodiments, a driver gene is a gene whole misexpression sustains the condition. In some embodiments, the driver gene is a gene provided herein below. In some embodiments, the driver gene is a gene provided in a Table. In some embodiments, the driver gene is a driver gene provided in a Table. In some embodiments, the Table is Table 3. In some embodiments, the Table is Table 4. In some embodiments, the Table is Table 6. In some embodiments, the driver gene is a gene provided in FIG. 17. In some embodiments, the driver gene is a gene selected from Vogelstein et al. (Vogelstein, B., et al., (2013, “Cancer Genome Landscapes.”, Science 339, 1546-1558), the pan-cancer or GBM-specific genes listed by Kandoth et al. (Kandoth, C., et al., 2013, “Mutational landscape and significance across 12 major cancer types.”, Nature 502, 333-339.), and 840 genes published by Verhaak et al., (Verhaak et al., 2010, “Integrated genomic analysis identifies clinically relevant subtypes of glioblastoma characterized by abnormalities in PDGFRA, IDH1, EGFR, and NF1.”, Cancer cell 17 (1): 98-110) the contents of which are all hereby incorporated by reference in their entirety.

In some embodiments, the driver gene is selected from ABL1, CASP8, DNMT1, EGFR, FGFR3, ACVR1B, AKT1, ALK, APC, AR, ARID1A, ARID1B, ARID2, ASXL1, ATM, ATRX, AXIN1, B2M, BAP1, BCL2, BCOR, BRAF, BRCA1, BRCA2, CARD11, CBL, CDC73, CDH1, CDKN2A, CDKN2C, CEBPA, CHEK2, CIC, CREBBP, CSFIR, CTNNB1, CYLD, DAXX, DNMT3A, EP300, ERBB2, EZH2, FBXW7, FGFR2, FLT3, FOXL2, FUBP1, GATA1, GATA2, GATA3, GNA11, GNAQ, GNAS, H3F3A, HNFIA, HRAS, IDH1, IDH2, JAK1, JAK2, JAK3, KDM5C, KDM6A, KIT, KLF4, KMT2C, KMT2D, KRAS, MAP2K1, MAP3K1, MED12, MEN1, MET, MLH1, MPL, MSH2, MSH6, MYD88, NCOR1, NF1, NF2, NFE2L2, NOTCH1, NOTCH2, NPM1, NRAS, PAX5, PBRM1, PDGFRA, PHF6, PIK3CA, PIK3R1, PPP2RIA, PRDM1, PTCH1, PTEN, PTPN11, RB1, RET, RNF43, RPL5, RUNX1, SETBP1, SETD2, SF3B1, SMAD2, SMAD4, SMARCA4, SMARCB1, SMO, SOCS1, SOX9, SPOP, SRSF2, STAG2, STK11, TET2, TNFAIP3, TP53, TRAF7, TSC1, TSHR, U2AF1, VHL, and WT1. In some embodiments, the driver gene is selected from ABL1, AKT1, AKT2, ASXL1, AXIN1, BCOR, BRCA2, CA12, CDKN2A, CHEK2, CHI3L1, CIC, CREBBP, DAXX, DLL3, DSCAML1, EGFR, EN1, ERBB2, FGF17, FGFR2, FGFR3, GATA1, GDF15, GNA11, GNAS, H3F3A, HK3, HRAS, KDM5C, KLF4, KMT2D, MBP, MEN1, MLH1, MYD88, NES, OLIG2, PBRM1, PDGFA, PDGFR1, PRDM1, RELB, SGCD, SMAD2, SMARCB1, SMO, SOCS1, SOX10, SOX9, SRSF2, STK11, TNFAIP3, TRAF7, VHL, VIPR2, AND ZIC2. In some embodiments, the driver gene is selected from ABL1, ACVRIB, AKT1, BCOR, BRCA1, CHEK2, CREBBP, CTNNB1, DAXX, DNMT3A, FBXW7, FGFR2, FUBP1, H3F3A, JAK1, KDM5C, KMT2D, MEN1, MLH1, MSH2, PBRM1, PRDM1, RNF43, SMAD2, SMO, SOCS1, SOX9, SRSF2, TNFAIP3, TRAF7, U2AF1, VHL, AR, CARD11, CASP8, CDKN2C, and MSH6.

In some embodiments, the driver gene is selected from AKT1, VHL, ABL1, AND BRCA1. In some embodiments, the driver gene is selected from SMAD2, RNF43, AKT1, VHL AND BCOR. In some embodiments, the driver gene is TNFAIP3. In some embodiments, the driver gene is selected from SMAD2 and RNF43. In some embodiments, the driver gene is selected from DAXX, CREBBP, ABL1, AKT1, FUBP1, BRCA1, FGFR2, SMAD2, VHL and CDKN2A. In some embodiments, the driver gene is JAK1. In some embodiments, the driver gene is selected from DAXX, ACVRIB, CREBBP, FUBP1, ABL1, AKT1, FGFR2, JAK1 and GNA11. In some embodiments, the driver gene is selected from CHEK2, DAXX, CREBBP, ABL1, AKT1, BRCA1, and FBXW7. In some embodiments, the driver gene is selected from CHEK2, DAXX, CREBBP, ABL1, AKT1, BRCA1, SMAD2, VHL, RNF43, FGFR2, ACVRIB, AXIN1, FUBP1, and JAK1.

In some embodiments, the measurements of DNA methylation are obtained from DNA from a biological sample from the subject. In some embodiments, the method comprises obtaining DNA from a biological sample from the subject. In some embodiments, the biological sample is selected from: tissue, blood, lymph, serum, cerebral spinal fluid, urine, breast milk, feces, saliva, tumor tissue and tumor fluid. In some embodiments, the tissue is a tumor biopsy. In some embodiments, the biological sample is blood.

In some embodiments, the DNA is genomic DNA. In some embodiments, the DNA is mitochondrial DNA. In some embodiments, the DNA is cDNA. In some embodiments, the DNA is cell free DNA (cfDNA). In some embodiments, the DNA is cancer cell free DNA (ccfDNA). In some embodiments, the DNA is cell free fetal DNA (cffDNA).

In some embodiments, the measurements of DNA methylation are obtained by obtaining DNA from a biological sample from the subject, isolating a plurality of cis-regulatory sequences from the obtained DNA and measuring DNA methylation within the plurality of isolated cis-regulatory sequences. In some embodiments, the method further comprises isolating a plurality of cis-regulatory sequences from the obtained DNA. In some embodiments, the method further comprises measuring DNA methylation within the plurality of isolated cis-regulatory sequences. In some embodiments, measurements of DNA methylation within cis-regulatory sequences of more than one potential driver gene are received. In some embodiments, measurements of DNA methylation within cis-regulatory sequences of a panel of potential driver genes are received. In some embodiments, a panel is at least 2, 3, 4, 5, 10, 15, 20, 25, 30, 35, 40, 45, 50, 60, 70, 75, 80, 90 or 100 potential driver genes.

In some embodiments, isolating comprises binding probes to the cis-regulatory sequences. In some embodiments, the isolating further comprises isolating the hybridized probes. In some embodiments, the probes are nucleic acid probes. In some embodiments, the probes are DNA probes. In some embodiments, the probes are RNA probes. In some embodiments, the probes are provided in Supplemental Table 3 of Edrei et al., 2021, “Methylation-mediated retuning of the enhancer-to-silencer activity scale of networked regulatory elements guides driver-gene misregulation”, doi.org/10.1101/2021.03.02.433521, herein incorporated by reference in its entirety. In some embodiments, a probe binds a protein indicative of the cis-regulatory sequence. In some embodiments, the probe binds chromatin bearing a protein wherein the chromatin is indicative of the cis-regulatory sequence. In some embodiments, the probe binds the cis-regulatory sequence. In some embodiments, the protein is a DNA-binding protein. In some embodiments, the protein is a histone. In some embodiments, the histone is a modified histone. In some embodiments, the modification is selected from methylation, acetylation, phosphorylation, sumoylation, and ubiquitination. In some embodiments, the histone is a histone variant. In some embodiments, the protein is H3. In some embodiments, the protein is H4. In some embodiments, a lysine of a histone is modified. In some embodiments, the lysine is selected from H3K4, H3K9, H3K14, H3K18, H3K23, H3K27, H3K36, H3K56, H3K79, H4K5, H4K8, H4K12, H4K16, and H4K20. In some embodiments, an arginine of a histone is modified. In some embodiments, the arginine is selected from H3R2, H3R17, and H4R3. In some embodiments, a serine of a histone is modified. In some embodiments, the serine is selected from H3S10, H3S28, and H4S1. In some embodiments, the modified histone is histone 3 lysine 4 monomethylation (H3K4me1). In some embodiments, the modified histone is H3K27 acetylation (H3K27ac). In some embodiments, the probes are nucleic acid probes. In some embodiments, the probes are DNA probes. In some embodiments, the probe binds the cis-regulatory sequence. In some embodiments, the probe binds the cis-regulatory sequence. In some embodiments, the probe is specific to the cis-regulatory sequence.

In some embodiments, the probe comprises a capture moiety. As used herein, a capture moiety is a molecule that can be isolated by binding to a capturing molecule. For example, the oligonucleotide can be conjugated to biotin (capture moiety) and then captured by a streptavidin column (the capturing molecule). Any capturing system may be used so that the polynucleotide can be isolated. In some embodiments, the capture moiety is a non-nucleic acid capture moiety. In some instances, the capture moiety comprises biotin, such that the nucleic acid molecule is biotinylated. In some instances, the capture moiety may comprise a capture sequence (e.g., nucleic acid sequence). In some instances, a sequence of the probe molecule may function as a capture sequence. In other instances, the capture moiety may comprise another nucleic acid molecule comprising a capture sequence. In some instances, the capture moiety may comprise a magnetic particle capable of capture by application of a magnetic field. In some instances, the capture moiety may comprise a charged particle capable of capture by application of an electric field. In some instances, the capture moiety may comprise one or more other mechanisms configured for, or capable of, capture by a capturing molecule. In some embodiments, the capture moiety is non-naturally occurring. In some embodiments, a probe comprising a capture moiety is non-naturally occurring. In some embodiments, the probe is a nucleic acid probe, and the capture moiety is a moiety not associated with nucleic acid molecules in nature. In some embodiments, the isolating comprises capturing the capture moiety to a capturing molecule. In some embodiments, the capturing molecule comprises avidin. In some embodiments, avidin is streptavidin.

In some embodiments, a plurality of cis-regulatory sequences is at least 2 cis-regulatory sequences. In some embodiments, a plurality of cis-regulatory sequences is at least 2, 3, 4, 5, 6, 7, 8, 9, or 10 cis-regulatory sequences. Each possibility represents a separate embodiment of the invention. In some embodiments, the plurality of cis-regulatory sequences regulates at least one potential driver gene. In some embodiments, the measurements are for at least two regulatory sequences that regulate a single gene. It will be understood by a skilled artisan that in order to determine a total regulatory effect for a gene there must be at least two regulatory sequences whose impact on the gene can be combined to generate the total effect. In some embodiments, the plurality of cis-regulatory sequences comprises at least 3 distinct cis-regulatory sequences. In some embodiments, the plurality of cis-regulatory sequences comprises at least 4 distinct cis-regulatory sequences.

In some embodiments, the cis-regulatory sequence comprises Histone 3 lysine 4 (H3K4) methylation. In some embodiments, methylation is mono-methylation. In some embodiments, the cis-regulatory sequence is marked by H3K4 methylation. In some embodiments, the cis-regulatory sequence is associated with histones comprising H3K4 methylation. In some embodiments, the cis-regulatory sequence comprises Histone 3 lysine 27 acetylation (H3K27ac). In some embodiments, the cis-regulatory sequence has variable H3K27 acetylation.

In some embodiments, the cis-regulatory sequence is not a promoter. In some embodiments, the cis-regulatory sequence is not in a promoter region. As used herein, the term “promoter” refers to the DNA sequence which is bound by the core transcriptional machinery to initiate transcription. In some embodiments, a promoter comprises the 100 bases upstream of the transcriptional start site (TSS) of the gene (−100 to −1 relative to the TSS). In some embodiments, a promoter comprises the 200 bases upstream of the transcriptional start site (TSS) of the gene (−200 to −1 relative to the TSS). In some embodiments, a promoter comprises the 300 bases upstream of the transcriptional start site (TSS) of the gene (−300 to −1 relative to the TSS). In some embodiments, a promoter comprises the 400 bases upstream of the transcriptional start site (TSS) of the gene (−400 to −1 relative to the TSS). In some embodiments, a promoter comprises the 500 bases upstream of the transcriptional start site (TSS) of the gene (−500 to −1 relative to the TSS). In some embodiments, a promoter comprises the 1000 bases upstream of the transcriptional start site (TSS) of the gene (−1000 to −1 relative to the TSS). In some embodiments, a promoter comprises the 1000 bases downstream of the transcriptional start site (TSS) of the gene (1000 to 0 relative to the TSS). In some embodiments, a promoter comprises the 500 bases downstream of the transcriptional start site (TSS) of the gene (500 to 0 relative to the TSS). In some embodiments, a promoter comprises the 400 bases downstream of the transcriptional start site (TSS) of the gene (400 to 0 relative to the TSS). In some embodiments, a promoter comprises the 300 bases downstream of the transcriptional start site (TSS) of the gene (300 to 0 relative to the TSS). In some embodiments, a promoter comprises the 200 bases downstream of the transcriptional start site (TSS) of the gene (200 to 0 relative to the TSS). In some embodiments, a promoter comprises the 100 bases downstream of the transcriptional start site (TSS) of the gene (100 to 0 relative to the TSS). In some embodiments, the promoter is the minimal promoter. In some embodiments, the promoter does not comprise enhancer elements. In some embodiments, the promoter does not comprise silencer elements.

In some embodiments, the cis-regulatory sequence is located within 1 megabase upstream or downstream of a transcriptional start site of a gene regulated by the cis-regulatory sequence. In some embodiments, a gene regulated by the cis-regulatory sequence is a potential driver gene. In some embodiments, the cis-regulatory sequence is not within 2 kb of a transcriptional start site of a gene regulated by the cis-regulatory sequence. In some embodiments, the cis-regulatory sequence is not within 2 kb up stream of a transcriptional start site of a gene regulated by the cis-regulatory sequence. In some embodiments, the cis-regulatory sequence is not within 1 kb up stream of a transcriptional start site of a gene regulated by the cis-regulatory sequence. In some embodiments, the cis-regulatory sequence is not within 50, 100, 150, 200, 250, 300, 400, 500, 600, 700, 750, 800, 900, 1000, 1250, 1500 or 2000 bases up stream of a transcriptional start site of a gene regulated by the cis-regulatory sequence. Each possibility represents a separate embodiment of the invention. In some embodiments, the promoter is defined by the above enumerated distances from the transcriptional start site.

In some embodiments, the cis-regulatory sequence is an enhancer element. In some embodiments, the cis-regulatory sequence is a repressor element. In some embodiments, the plurality of cis-regulatory sequences is selected from enhancer and repressor elements. In some embodiments, the plurality of cis-regulatory sequences comprises at least one repressor element. In some embodiments, the plurality of cis-regulatory sequences comprises at least one enhancer element. In some embodiments, a cis-regulatory sequence comprises at least one CpG dinucleotide. In some embodiments, a cis-regulatory sequence comprises a plurality of CpG dinucleotides. In some embodiments, a cis-regulatory sequence comprises more than one CpG dinucleotide. In some embodiments, the cis-regulatory sequences are located between genomic positions provided in Table 3. In some embodiments, the cis-regulatory sequences are located in the genomic intervals provided in Table 3. In some embodiments, the cis-regulatory sequences are located between genomic positions provided in Table 4. In some embodiments, the cis-regulatory sequences are located in the genomic intervals provided in Table 4.

In some embodiments, an activator is selected from RNAP, GATA2, GATA3, EP300, BCL3, NFATC1, HNF4A, HNF4G, ELK4, ELK1 and IRF1. In some embodiments, a repressor is selected from REST, YY1, ZBTB33, SUZ12, EZH2, RCOR1, CTCF, SMC3, RAD21, PAX5 and RUNX3

In some embodiments, the regulatory effect of a cis-regulatory sequence is determined independently. In some embodiments, the regulatory effects of at least two cis-regulatory sequences are determined separately. In some embodiments, the regulatory effect of a cis-regulatory sequence is determined in combination with at least one other cis-regulatory sequence. In some embodiments, the regulatory effect of each cis-regulatory sequence is determined independently. In some embodiments, the regulatory effect of each cis-regulatory sequence is determined in combination with at least one other cis-regulatory sequence. In some embodiments, the regulatory effect of a plurality of cis-regulatory sequences are determined together. In some embodiments, the measured regulatory effects are summed to produce the total regulatory effect. In some embodiments, the regulatory effects of at least two cis-regulatory sequences are determined separately and summed to produce the total regulatory effect. In some embodiments, the regulatory effect of the plurality of cis-regulatory sequences are each determined separately and summed to produce the total regulatory effect. In some embodiments, the total regulatory effect for at least two cis-regulatory sequences is determined simultaneously. In some embodiments, the total regulatory effect for at least two cis-regulatory sequences is determined in combination.

In some embodiments, at least one measured cis-regulatory sequence comprises more than one CpG dinucleotide. In some embodiments, a measurement from at least one CpG dinucleotide within the cis-regulatory sequence is received. In some embodiments, a measurement from at least one of the plurality or more than one CpG dinucleotide within the cis-regulatory sequence is received. In some embodiments, the methylation status of the CpG dinucleotide is measured. In some embodiments, methylation of the cystine in the CpG dinucleotide is measured.

In some embodiments, the determining comprises testing each of the plurality of cis regulatory sequences. In some embodiments, the testing produces a measure of a regulatory effect of the sequences. In some embodiments, the measure is a magnitude. In some embodiments, a positive magnitude is an enhancing effect. In some embodiments, a negative magnitude is a silencing effect. In some embodiments, effect is a transcriptional effect. In some embodiments, the test is an expression assay. In some embodiments, the test measures expression. In some embodiments, expression is expression of a coding sequence. In some embodiments, the assay measures regulatory effect of a cis-regulatory sequence. In some embodiments, effect is effect on expression of a coding sequence. In some embodiments, expression is transcription. In some embodiments, a coding sequence is a control coding sequence. In some embodiments, a coding sequence is an irrelevant coding sequence. In some embodiments, a coding sequence is a detectable coding sequence. In some embodiments, a coding sequence is a test coding sequence. In some embodiments, the coding sequence is not expressed in a cell used for the assay. In some embodiments, the coding sequence is not expressed in a cell used for the testing. In some embodiments, the testing comprises testing methylated and unmethylated copies of the plurality of cis-regulatory sequences. In some embodiments, copies of the plurality are copies of each of the plurality of cis-regulatory sequences. In some embodiments, the tested regulatory effect is used to produce the total regulatory effect. In some embodiments, the tested regulatory effect is summed to produce the total regulatory effect.

In some embodiments, determining comprises comparing the received measurements to a database. In some embodiments, the database comprises potential driver genes, methylation status of at least one cis-regulatory sequences of a database gene, and regulatory effects of the cis-regulatory sequence on the database gene. In some embodiments, the database comprises potential driver genes, methylation status of a plurality of cis-regulatory sequences of a database gene, and regulatory effects of the plurality of cis-regulatory sequence on the database gene. In some embodiments, the database comprises potential driver genes, methylation status of cis-regulatory sequences of a database gene, and regulatory effects of the cis-regulatory sequences on the database gene. In some embodiments, the database comprises the regulatory effect of individual cis-regulatory sequences. In some embodiments, the database comprises a combined regulatory effect of a plurality or more than one cis-regulatory sequence.

In some embodiments, determining comprises applying a machine learning algorithm to the received measurements. In some embodiments, the machine learning algorithm is or has been trained on cis-regulatory sequences with known methylation status. In some embodiments, the machine learning algorithm is or has been trained on cis-regulatory sequences with known regulatory effect on a driver gene. In some embodiments, the machine learning algorithm is or has been trained on cis-regulatory sequences with known methylation status and known regulatory effect on a driver gene.

Machine learning is well known in the art, and by performing the methods of the invention on cis-regulatory sequences with known methylation status and known regulatory effect the machine learning algorithm can learn to recognize total regulatory effect based on methylation status. In some embodiments, at least 1, 2, 3, 4, 5, 6, 7, 8, 9, 10, 15, or 20 cis-regulatory sequences are analyzed before the algorithm can identify the total regulatory effect on a given gene.

In some embodiments, the machine learning algorithm has been trained on single cis-regulatory sequences. In some embodiments, the machine learning algorithm has been trained on genes and at least one of each gene's cis-regulatory sequences. In some embodiments, the machine learning algorithm has been trained on genes and a plurality of each gene's cis-regulatory sequences. In some embodiments, the machine learning algorithm has been trained on genes and all of each gene's cis-regulatory sequences.

In some embodiments, the predetermined threshold is derived from a predetermined standard regulatory effect for the cis-regulatory sequences of the at least one potential driver gene. In some embodiments, the predetermined standard regulatory effect is determined in cells grown in culture. In some embodiments, the predetermined standard regulatory effect is determined in cells from a healthy subject. In some embodiments, the predetermined standard regulatory effect is determined in cells from a subject suffering from a pathological condition.

In some embodiments, the method further comprises confirming aberrant expression of the selected driver gene in a sample. In some embodiments, the sample is from the subject. In some embodiments, the method further comprises measured expression of the selected driver gene in a sample. In some embodiments, the method further comprises administering a therapeutic agent that targets the selected driver gene. In some embodiments, the method further comprises administering a therapeutic agent that treats the selected driver gene. In some embodiments, the method further comprises administering a therapeutic agent that targets DNA methylation. In some embodiments, the method further comprises administering a therapeutic agent that targets DNA methylation machinery. In some embodiments, the targeted DNA methylation is methylation in cis-regulatory sequences. In some embodiments, the targeted DNA methylation is methylation in cis-regulatory sequences of a target driver gene.

In some embodiments, a potential driver gene is selected from the genes provided in Table 3. In some embodiments, a potential driver gene is a gene selected from the genes provided in Table 3. In some embodiments, a potential driver gene is any one of the genes provided in Table 3. In some embodiments, a potential driver gene is selected from the driver genes provided in Table 3. In some embodiments, a potential driver gene is a gene selected from the driver genes provided in Table 3. In some embodiments, a potential driver gene is any one of the driver genes provided in Table 3. In some embodiments, a potential driver gene is selected from Table 4. In some embodiments, a potential driver gene is a gene selected from Table 4. In some embodiments, a potential driver gene is any one of the genes provided in Table 4. In some embodiments, a potential driver gene is selected from Table 5. In some embodiments, a potential driver gene is a gene selected from Table 5. In some embodiments, a potential driver gene is any one of the genes provided in Table 5. In some embodiments, a potential driver gene is selected from a driver gene in Table 5. In some embodiments, a potential driver gene is a driver gene selected from Table 5. In some embodiments, a potential driver gene is any one of the driver genes provided in Table 5. In some embodiments, the condition is glioblastoma, and a potential driver gene is selected from a gene in Tables 3, 4 and 5. In some embodiments, the condition is glioblastoma, and a potential driver gene is selected from a driver gene in Tables 3 and 5. In some embodiments, the panel comprises at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, or 125 driver genes. Each possibility represents a separate embodiment of the invention. In some embodiments, the panel comprises at most, 20, 25, 30, 40, 50, 60, 70, 75, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 2000, 3000, 4000, 5000 or 10000 driver genes. Each possibility represents a separate embodiment of the invention.

In some embodiments, total regulatory effect on a panel of driver genes are determined. In some embodiments, the total regulatory effect is determined for each driver gene of the panel. In some embodiments, the panel is selected from the genes provided in Table 3. In some embodiments, the panel is selected from the genes provided in Table 4. In some embodiments, the panel is selected from the genes provided in Table 5. In some embodiments, the panel is selected from the driver genes provided in Table 3. In some embodiments, the panel is selected from the driver genes provided in Table 4. In some embodiments, the panel is selected from the driver genes provided in Table 5. In some embodiments, the panel comprises the genes provided in Table 5. In some embodiments, the panel comprises the driver genes provided in Table 3. In some embodiments, the panel comprises the driver genes provided in Table 4. In some embodiments, the panel consists of the driver genes provided in Table 5. In some embodiments, the panel consists of the driver genes provided in Table 4. In some embodiments, the panel consists of the driver genes provided in Table 3.

In some embodiments, the method of the invention is for use in diagnosing a pathological condition. In some embodiments, the method of the invention is for use in diagnosing increased risk of developing a pathological condition. In some embodiments, the method of the invention is for use in determining increased risk of developing a pathological condition.

By another aspect, there is provided a kit comprising probes that hybridize to cis-regulatory sequences of a plurality of target genes.

In some embodiments, the probes are protein probes. In some embodiments, the probes a nucleic acid probes. In some embodiments, the probes are nucleotide probes. In some embodiments, the nucleic acid is DNA. In some embodiments, the nucleic acid is RNA. In some embodiments, the probes are at least 10, 12, 15, 17, 20, 25, or 30 nucleotides in length. Each possibility represents a separate embodiment of the invention. In some embodiments, the probe comprises a capture moiety.

In some embodiments, the kit comprises at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 150, 200, 250, 300, 350, 375, 400, 450, 500, 600, 700, 750, 800, 900 or 1000 probes. Each possibility represents a separate embodiment of the invention. In some embodiments, the kit comprises at most, 20, 25, 30, 40, 50, 60, 70, 75, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 2000, 3000, 4000, 5000, 10000, 15000, 20000, 25000, 30000, 35000, 38000, 38077, 38100, 39000, 40000, 45000, 50000, 60000, 70000, 80000, 90000, or 100000 probes. Each possibility represents a separate embodiment of the invention.

In some embodiments, the probes are selected from the probe sequences provided in SEQ ID NO: 28-38077. In some embodiments, the probes comprise sequences from SEQ ID NO: 28-38077. In some embodiments, the probes comprise SEQ ID NO: 28-38077. In some embodiments, the probes consist of SEQ ID NO: 28-38077.

In some embodiments, the target gene is a potential driver gene. In some embodiments, the target gene is a gene provided hereinabove. In some embodiments, the cis-regulatory sequences are sequences provided hereinabove. In some embodiments, the kit further comprises a capturing molecule.

In some embodiments, the kit of the invention is for use in diagnosing a pathological condition. In some embodiments, the kit of the invention is for use is prognosing a pathological condition.

By another aspect, there is provided a computer program product for determining a driver gene for a pathological condition, comprising a non-transitory computer-readable storage medium having program code embodied thereon, the program code executable by at least one hardware processor to:
a. receive measurements of DNA methylation within a plurality of cis-regulatory sequences;
b. determine from the received measurements a total regulatory effect of cis-regulatory sequences upon at least one potential driver gene of the pathological condition; and
c. select the at least one potential driver gene as a driver of the pathological condition when the total regulatory effect is beyond a predetermined threshold.

In some embodiments, the computer program product is for performing a method of the invention. In some embodiments, the computer program product is for determining a driver gene of a pathological condition.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Overall Research-Flow and Terminology

Herein, the term “gene domains” refers to 2 MB genomic windows centered at the Transcription Start Sites (TSSs) of the targeted genes. Within these windows, blocks of chromatin were located which showed variable levels of regulatory activity across the studied GBM tumors. RNA probes (120 bp each) were designed to capture the CpG methylation sites within these chromatin blocks. Genomic tumor DNAs were arbitrarily sheared using a sonication device into collections of DNA fragments of various sizes. Throughout, these fragments are referred to as “DNA Segments”. These DNA segments were then allowed to attach the RNA probes, which fully or partially overlapped their span. The resulting collection of Captured DNA Segments (median size=224 bp) was integrated into gene-reporting vectors or underwent regular or methylation sequencing.

Following, the regulatory outputs of contiguous segments, captured by contiguous probes, were analyzed, and Transcriptional Activity Scores (TASs) were calculated in 500 bp (50% overlapping) windows along the targeted regions. This process revealed functional “regulatory elements” (i.e., methylation-sensitive and methylation-insensitive enhancers and silencers), of them 26,152 showed FDR q value <0.05. The above experiments were used to elucidate the basic roles of methylation effects on enhancers and silencers under simplified genomic arrangements and extreme methylation or unmethylation conditions.

Based on this understanding, actual tumor chromatins were studied. It was found that clusters of gene-associated methylation sites formed defined “regulatory units” of tens to thousands (average 834, median 333) bp-long spans, containing homogenous (positive or negative), contiguous gene-associated methylation sites. Each of these units mediate positive or negative input to the transcription of a particular gene (Table 5). Note that these regulatory units are learned features of the GBM genome, as no pre-assumptions regarding the size or organization of the units were applied.

### GBM Samples and Data

Tumor biopsies and associated clinical data were collected and encoded at the DKFZ Institute, Heidelberg, Germany. Whole-genome and whole-exome, H3K4me1 and H3K27ac chromatin immunoprecipitation (GSE121719) and RNA sequencing of the GBM biopsies and the normal brain samples (GSE121720), and the analyses of coding DNA mutation, gene expression and DNA copy number variation, were performed at the DKFZ. Encoded de-personalized DNA samples and data were used as input materials for target enrichment of gene regulatory regions and associated DNA methylation and non-coding DNA mutation analyses, which were performed at the Hebrew University, Jerusalem, Israel (HUJI).

### Genes

Genes analyzed in the study included the pan-cancer driver genes listed by Vogelstein et al. (Vogelstein, B., et al., 2013b, “Cancer Genome Landscapes.”, Science 339, 1546-1558, herein incorporated by reference in its entirety) and the pan-cancer or GBM-specific driver genes listed by Kandoth et al. (Kandoth, C., et al., (2013)., “Mutational landscape and significance across 12 major cancer types.” Nature 502, 333-339, herein incorporated by reference in its entirety), but excluding the HIST1, H3B and CRLF2 genes due to missing expression data, and the AMERI gene for which probe design failed. Cancer type-specific genes (n=23) were selected from a published list of 840 genes (Verhaak et al., 2010, “Integrated genomic analysis identifies clinically relevant subtypes of glioblastoma characterized by abnormalities in PDGFRA, IDH1, EGFR, and NF1”, Cancer cell 17 (1): 98-110, herein incorporated by reference in its entirety). Non-driver variable genes (n=22) were defined as those showing top expression variation among the 70 analyzed GBM samples for which there was found at least two correlative sites in the TCGA-GBM dataset. The genomic coordinates for gene features from the hg19 refGene table of the UCSC Genome Browser were used.

### Public Databases

The Cancer Genome Atlas (TCGA): Gene expression (RNAseqV2 normalized RSEM) and DNA methylation data (HumanMethylation450) were download in May 2019 using TCGAbiolinks for the following cancer types: BRCA (778 genomes), CESC, (304), COAD (306), ESCA (161), GBM (50), KICH (65), KIRC (320), KIRP (273), LIHC (371), LUAD (463), PAAD (177), SKCM (103), THYM (119).

NIH Roadmap Epigenomic Project: H3K4me1 broad peaks of corresponded TCGA tumor types and DNasel cell specific narrow peaks of normal brain (E081 and E082).

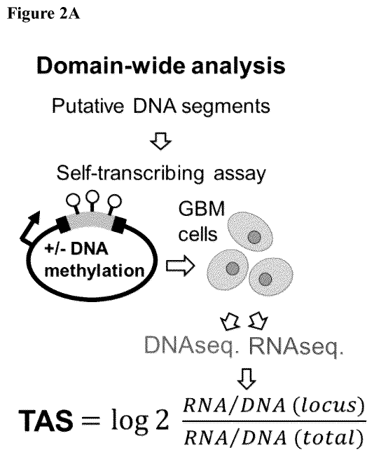

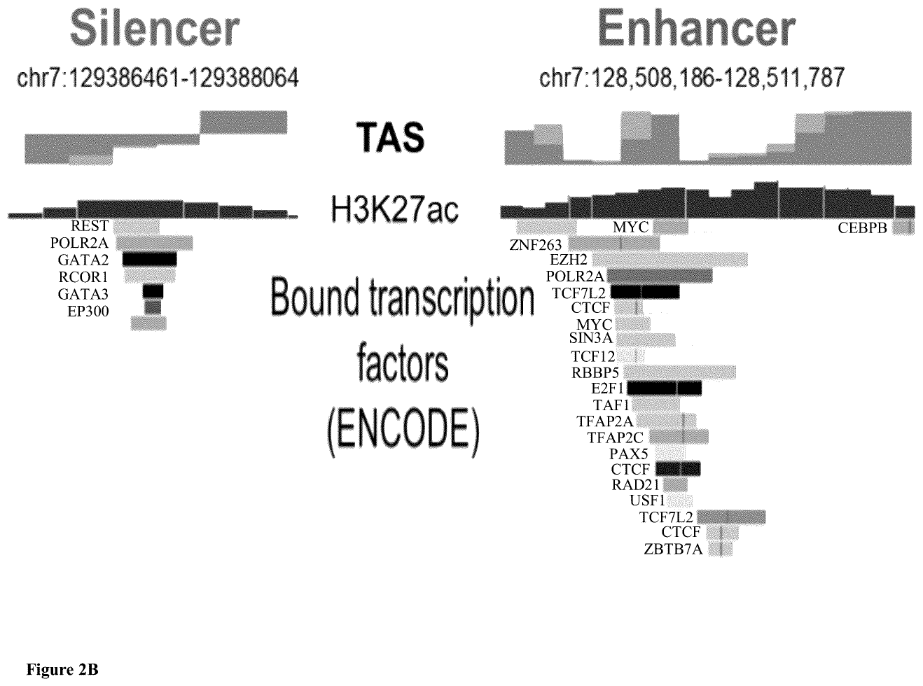

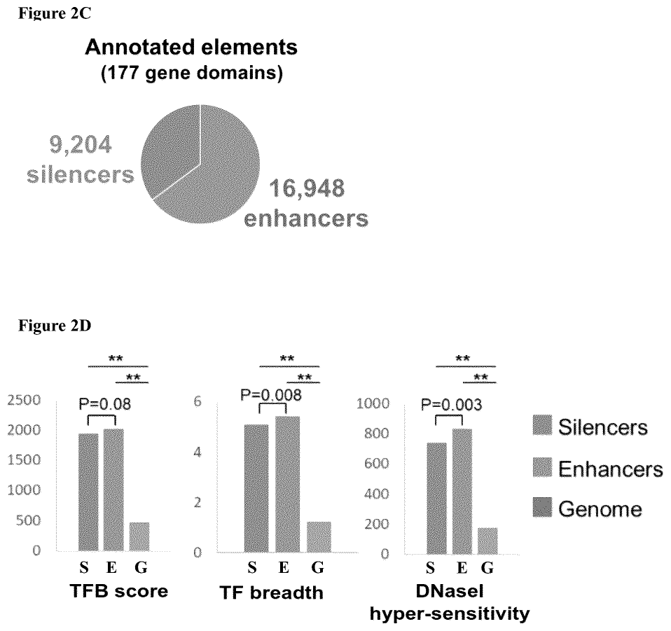

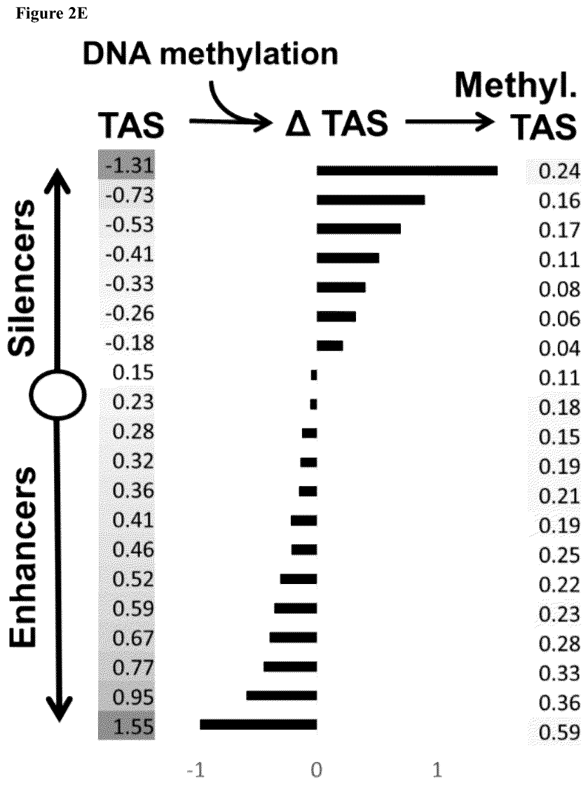

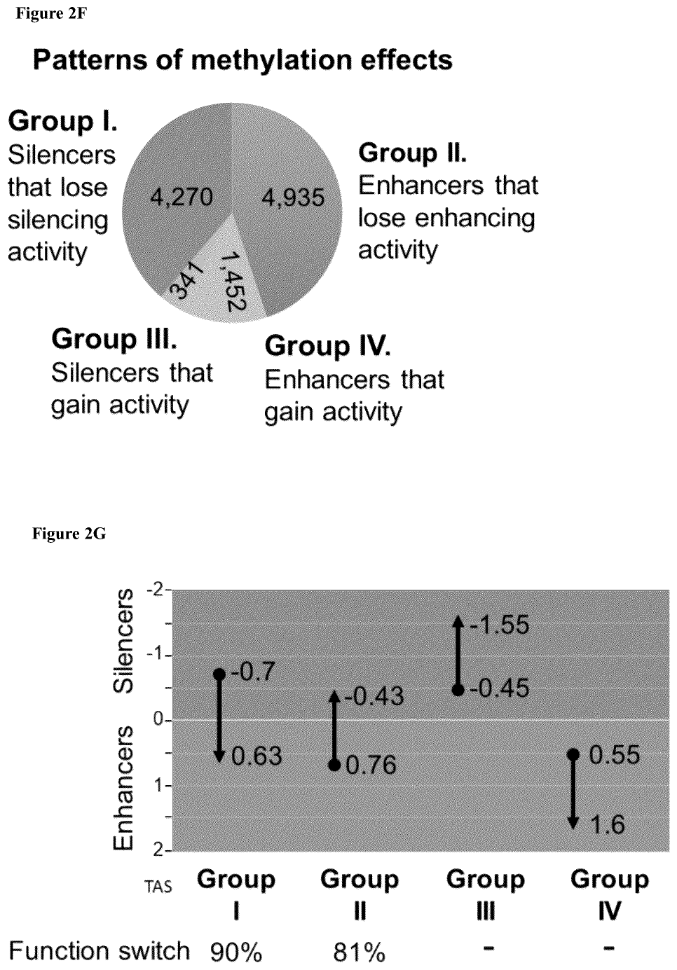

Encyclopedia of DNA Elements (ENCODE): DNasel hypersensitivity peak clusters (wgEncodeRegDnaseClusteredV3.bed.gz) and transcription factor ChIP-seq clusters (wgEncodeRegTfbsClusteredWithCellsV3.bed.gz) and DNase brain tumors data (Gliobla and SK-N-SH). The ENCODE transcription factor binding (TFB) scores presented in FIG. 2 represent the peaks of transcription factor occupancy from uniform processing of ENCODE ChIP-seq data by the ENCODE Analysis Working Group. Scores were assigned to peaks by multiplying the input signal values by a normalization factor calculated as the ratio of the maximum score value (1000) to the signal value at one standard deviation from the mean, with values exceeding 1000 capped at 1000. Peaks for 161 transcription factors in 91 cell types are combined here into clusters to produce a summary display showing occupancy regions for each factor and motif sites within the regions when identified. One-letter code for the different cell lines is given in hgsv.washington.cdu/cgi-bin/hgTrackUi?hgsid=2654998_09Di2gB797ixpn70898j4DsMV3Ro&g=wgEncodeRegTf bsClusteredV3.

Additional public data: HiC Data for TADs were downloaded from wangftp.wustl.edu/hubs/johnston_gallo/.

### Cell Lines

Human GBM T98G cells were purchased from the ATCC collection (ATCC® CRL-1690™), and cultured in minimum essential medium-Eagle #01-025-1A (Biological Industries), supplemented with 10% heat-inactivated FBS #04-127-1A (Biological Industries), 1% penicillin/streptomycin P/S #03-031-1B (Biological Industries), 1% L-glutamine #03-020-1C (Biological Industries;), 1% non-essential amino acids, #01-340-1B (Biological Industries) and 1% sodium pyruvate #03-042-1B (Biological Industries), at 37° C. and 5% CO₂.

### Target Enrichment Assays

Variable regulatory regions were defined as the regions carrying H3K4me1 marks in all tumors, and also H3K27ac in at least 25% of the tumors, but not in at least another 25% of the tumors. RNA probes were designed to target methylation sites within these regions, utilizing the SureDesign tool (earray.chem.agilent.com/suredesign/). Probe duplication was applied in cases (n=8,652) of >5 CpG sites within the 120 bp span of the probes. Repetitive regions were identified by BLAT and excluded from the design. Custom-designed biotinylated RNA probes were ordered from Agilent Technologies (agilent.com). The probe sequences are provided in SEQ ID NO: 28-38077.

Genomic tumor DNAs were arbitrarily sheared using a sonication device into collections of DNA fragments of various sizes. These DNA segments were then allowed to attach the probes which fully or partially overlapped their span. The resulting collection of captured DNA segments (median size=224 bp) was integrated into gene-reporting vectors or underwent sequencing.

Enrichment libraries of GBM-targeted regulatory DNA segments were constructed using the SureSelect #G9611A protocol (Agilent) for Illumina multiplexed sequencing, which used 200 nanograms genomic DNA per reaction, or the SureSelect Methyl-Seq #G9651A protocol using 1 microgram genomic DNA per reaction. Quality and size distribution of the captured genomic segments were verified using the TapStation nucleic acids system (Agilent) assessments of regular or bisulfite-converted libraries. Target enrichment efficiency and coverage was evaluated via sequencing.

### Massively Paralleled Reporter Assay

Massively parallel functional assays were performed as described (Arnold et al., 2013, “Genome-wide quantitative enhancer activity maps identified by STARR-seq”, Science 339 (6123): 1074-1077, herein incroporated by reference in its entirety), with the following modifications:
1) Reporter backbone: The pGL3-Promoter #E1761 GenBank accession number U47298 backbone (Promega) was used as a screening vector. The vector was modified as follows: The sequence between the Sacl and the Afel sites in the original pGL3-promoter vector (Promega, GenBank accession number U47298) was replaced with synthetic sequence. The modified vector produced a certain amount of basal transcription when no regulatory elements were presented. To evaluate regulatory functionality, putative silencer or enhancer elements were incorporated between the Agel and the Sall sites.
2) Genomic inputs: Plasmid libraries were constructed using a target-enriched library as input materials: One microliter of adaptor-ligated DNA fragments from the AK100 target enrichment library was amplified in eight independent PCR reactions, using KAPA Hifi Hot Start Ready Mix #KK2601 (KAPA Biosystems). Reaction conditions included 45 seconds(s) at 95° C., 10 cycles of 15s at 98° C., 30s at 65° C., 30s at 72° C., and 2 min final extension at 72° C., applying forward Ilumina universal primer: 5′-TAGAGCATGCACCGGTAATGATACGGCGACCACCGAGATCT-3′ (SEQ ID NO: reverse Indexed 1) and Ilumina primer: 5′-GGCCGAATTCGTCGACCAAGCAGAAGACGGCATACGAGAT-3′ (SEQ ID NO: 2), containing Illumina adapter sequences. A specific 15nt extension was added to each adapter as homology arms for directional cloning. PCR reactions were pooled and purified on NucleoSpin Gel and PCR Clean-up #740609 columns (Macherey-Nagel). The screening vector was linearized with Agel-HF and Sall-HF restriction enzymes (NEB) and purified through electrophoresis and gel extraction. Purified PCR products were cloned into the linearized vector by recombination with the adaptor-ligated homology arms in 12 reactions of 10 μl each, applying the In-Fusion HD #639649 kit (Clontech). The reactions were then pooled and purified with 1× Agencourt AMPureXP #A63881 DNA beads (Beckman Coulter) and eluted in 24 μl nuclease-free water.
3) Library propagation: Aliquots (n=12, 20 μl each) of MegaX DH10B TI Electrocomp bacteria #C640003 (Invitrogen) were transformed with 2 μl of the plasmid DNA library, according to the manufacturer's protocol, except for the electroporation step, which was performed using the Nucleofactor 2b platform (Lonza) Bacteria program 2. Every three transformation reactions were pooled (total of 4 reactions) for a one-hour recovery at 37° C., in SOC medium, while shaking at 225 rpm, after which, each reaction was transferred to 500 ml LBAMP (Luria Broth Ampicillin) for overnight 37° C. incubation, while shaking at 225 rpm. Propagated plasmid libraries were extracted using NucleoBond Xtra Maxi Plus Kit (#740416) (MAcherey-Nagel). To verify unbiased amplification of the targeted genomic segments, size distribution and coverage of the library were analyzed before and after the propagation step.
4) In-vitro methylation assay: Complete de-methylation stages were achieved by propagation of the libraries in bacteria following PCR amplification stages. In-vitro methylation of the de-methylated plasmid DNA was performed using the New England Biolabs CpG Methyltransferase M.Sssl #M0226M according to the manufacturer's instructions. Efficient methylation level was confirmed by using a DNA protection assay against FastDigest Hpall #FD0514 (Thermo Scientific) digestion.
5) Transfection to GBM cells: 20 μg of DNA were transfected into 2×10∧6 T98G and U87 cells at 70-80% confluence, using the Lipofectamine 3000 transfection kit #L3000-015 (Invitrogen), according to the manufacturer's protocol. In each experiment, 5×10∧7 T98G cells were transformed and incubated at 37° C., for 24 h.
6) Isolation of plasmid DNA and RNA from GBM cells: Plasmid DNA was extracted from 2.5×10∧7 cells, 24 h post-transfection. Cells were rinsed twice with PBS pH 7.4 using the NucleoSpin Plasmid EasyPure kit #740727250 (Macherey-Nagel), according to the manufacturer's protocol. Total RNA was extracted from 2.5×10∧7 cells 24h post-transfection using GENEZOL reagent #GZR200 (Geneaid), according to the manufacturer's protocol. The polyA+RNA fraction was isolated using Dynabeads Oligo-(dT) 25 #61002 (Thermo scientific), scaling up the manufacturer's protocol 5-fold per tube, and treated with 10 U turboDNase #AM2238 (Invitrogen) at 20 ng/μl 37° C., for 1 h. Two reactions of 50 μl each, were pooled and subjected to RNeasy MinElute #74204 reaction clean up (Qiagen) to inactivate turbo DNase and concentrate the polyA+RNA.
7) Reverse transcription: First strand cDNA synthesis was performed with 1-1.5 μg polyA+RNA in a total of 4 reactions of 20 μl each, using the Verso cDNA Synthesis Kit #AB1453B (Thermo scientific) at 42° C. for 30 min, 95° C. for 2 min, with a reporter-RNA specific primer (5′-CAAACTCATCAATGTATCTTATCATG-3′, (SEQ ID NO: 3)). cDNA (50 ng) was amplified by PCR, at 98° C. for 3 min, followed by 15 cycles at 98° C. for 20s each, 65° C. for 15s, 72° C. for 30s. Final extension was performed at 72° C. for 2 min, using Hifi Hot Start Ready Mix (KAPA), with reporter-specific primers. Forward primer: 5′-GGGCCAGCTGTTGGGGTG*T*C*C*A*C-3′ (SEQ ID NO: 4) which spans the splice junction of the synthetic intron and reverse primer: 5′-CTTATCATGTCTGCTCGA*A*G*C-3′ (SEQ ID NO: 5), where “*” indicates phosphorothioate bonds. In total, 16-20 reactions were performed. The amplified products were purified with 0.8× AMPureXP DNA beads (Agencourt) and eluted in 20 μl nuclease-free water. The resultant purified products served as a template for a second PCR performed under the following conditions: 98° C. for 3 min, 12 cycles of 98° C. for 15s, 65° C. for 30s, 72° C. for 30s. Final extension was performed at 72° C. for 2 min, with forward Ilumina universal primer: 5′-TAGAGCATGCACCGGTAATGATACGGCGACCACCGAGATCT-3′ (SEQ ID NO: 1) and reverse Indexed Ilumina primer: 5′-GGCCGAATTCGTCGACCAAGCAGAAGACGGCATACGAGAT-3′ (SEQ ID NO: 2). PCR products were purified with 0.8× AMPureXP DNA beads (Agencourt), eluted in 10 μL nuclease-free water, and pooled.

### Transcriptional Activity Analysis

Quality and size distribution of extracted plasmid DNAs and RNAs were verified using TapStation. DNA and cDNA samples were sequenced using the HiSeq2500 device (Illumina), as per the 125 bp paired-end protocol. Alignment with the hg 19 reference genome was performed on the first 40 bp from both sides of the DNA segments, using Bowtie2. Reads with mapping quality value above 40 aligned with the probe targets were considered for further analyses. Each of the captured genomic segments was given a unique ID according to genomic location and indicated the total number of DNA and RNA reads. Only on-target segments with at least one RNA read (n=623,223 pre-methylation; 304,998 post-methylation) were included. >99% of the targeted regions were presented following the propagation in bacteria and re-extraction from T98 cells. Technical and biological replications performed using illumina MiSeq sequencing.

Transcriptional activity score (TAS) was calculated as follows:
TAS=log₂((RNAj/DNAⱼ)/(RNAₜₒₜₐₗ/DNAₜₒₜₐₗ)),
where j is a genomic element and RNAₜₒₜₐₗ or DNAₜₒₜₐₗ are the sum of all segment reads.

For the analyses of isolated regulatory elements, TAS was determined in 500 bp, 50% overlapping windows, across the genome, based on DNA and RNA reads of segments overlapping with the given window. TAS significance was tested by Chi-square against total RNA to DNA. Multiple comparisons were corrected by applying False Discovery Rate (FDR). Functional regulatory elements were defined as elements with FDR q value <0.05 and minimum 100 RNA reads, where positive TASs were defined as enhancers, and negative as silencers. The methylation effect was analyzed by calculating TAS difference between treatments, where regulatory elements with a difference of ≥1.5-fold activity were counted.

### Inferring Cis-Regulatory Circuits

Methylation sequencing: Methyl-seq-captured libraries were sequenced using a Hiseq2500 device (Illumina), by applying paired-end 125 bp reads. Sequence alignment and DNA methylation calling were performed using Bismark VO.15.0 software against the hg19 reference genome. The sequencing yielded 52-149 million reads per sample, at an average mapping efficiency of 78.1%, average bisulfite efficiency of 97.6%, and 99.4% on target average. Overall, a mean coverage of 916 reads per site was obtained, and 86% of the targeted sites were covered by at least 100 reads. Sites that appeared in less than eight of the tumors were excluded from the analyses.

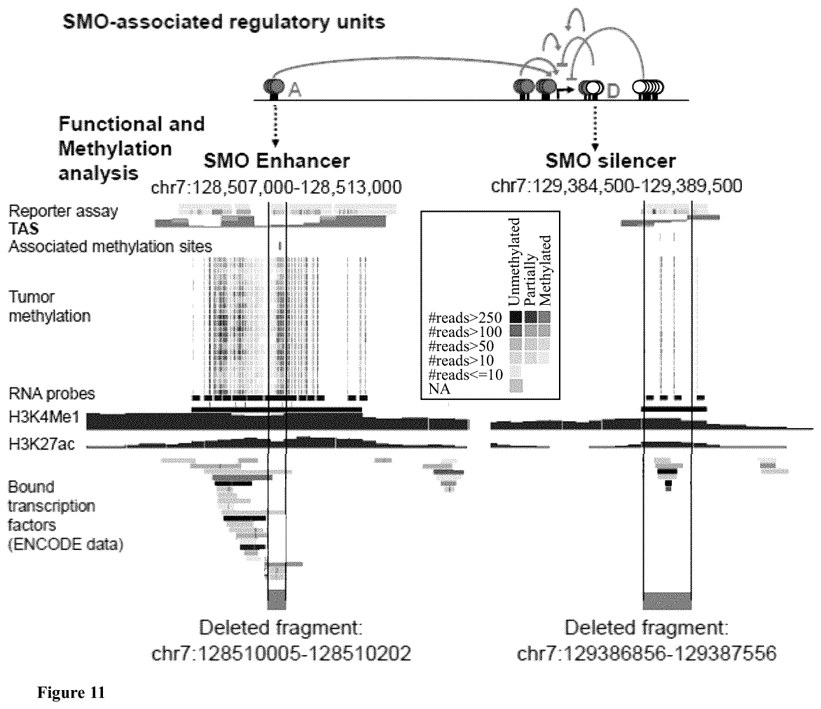

Circuit annotation: Correlation between the expression level of each targeted gene and the DNA methylation level of targeted CpG sites in a 2Mbp region flanking its transcription start site (TSS), was assessed by applying pairwise Spearman's rank correlation coefficient with Benjamini-Hochberg correction for multiple-hypothesis testing at an FDR <5%. Circuits with R2 >0.3 were included. Sites that correlated (R2 >0.1) with expression of the PTPRC (CD45) pan-blood cells marker, were considered a possible result of blood contamination and were eliminated from later analyses. Potential secondary effects were considered in two cases. (1) The correlated site was included within the prescribed portion (the gene body, excluding the first 5Kbp) of another gene; (2) The correlated site was located within the promoter (from TSS-1500 bp to TSS+2500 bp) of another gene. For these cases, correlation between the expression level of the genes was tested, and circuits with R2>0.1 that fit one of the scenarios described in FIG. 11, were excluded. For model developing, circuits which mismatched the report assay: circuits with methylation sensitive TAS (which were calculated for the DNA segments overlapping the given site and were changed by×1.5 fold by methylation) which mismatched the canonical mode (i.e., gropes I and II in FIG. 2F) were excluded.

Methylation-based prediction of gene expression: For each gene, two methods were performed (1) multiple linear regression and (2) Lasso regression. (1) Multiple linear regression should reduce the number of variables since there are only 24 samples. Thus, all the possible combinations of one to four associated sites were tested. For each combination with full data in at least 12 tumors, a predictive model of expression level based on multiple linear regression of the sites methylation levels was generated. A significant model (q value <0.05), evaluated by ANOVA for Linear Model Fit, and corrected for the number of possible models per-gene by FDR, was considered. A gene was considered to have a synergic model if the predictive value of the model was better than each of the involved sites alone.

Validation of methylation-based predictions was performed using the leave-one-out cross validation approach for assessing the generalization to an independent data set. One round of cross-validation involves 23 data sets (called training set) in which performing all the analysis, and one sample for validating the analysis (called testing set). The cross-validation was performed ×24 times. For each training data set, cis-regulatory circuits were generated (as described in Circuit annotation sub-section hereinabove) and possible predictive models were developed for the targeted genes. Prediction quality of each gene was then tested in the 24 rounds, by comparing predicted versus observed expression level. Difference up to 2-fold were considered as success. The ability to accurately predict the expression level of a gene was considered verified if it has good prediction quality in at least 20 of the 24 rounds.

### Analysis of Coding Sequence Variations

VCF files describing single nucleotide variations (SNV) were provided by the DKFZ. Synonymous SNV, SNVs overlapping with published SNPs (COMMON), or SNVs with a less than 25-read coverage or bcftools-QUAL score >20, were excluded. Copy number variations (CNV) were analyzed by whole-genome sequencing (WGS) data provided by the DKFZ. Association between gene expression and copy number was evaluated by Pearson or Spearman's correlations. p-values were adjusted for multiple-hypothesis testing using the Benjamini-Hochberg method, with FDR <5%.

### Analysis of Regulatory Sequence Variations

Pre-alignment processing: GBM tumors (n=8) were sequenced using the paired-end 250- or 300 bp read protocol on Illumina MiSeq V2 or V3 devices. FASTQ files were filtered, and sequence edges of Phred score quality >20 and trimmed up to 13 bp of Illumina adapter applying Trim Galore (bioinformatics.babraham.ac.uk/projects/trim_galore/). Reads that were shortened to 20 bp or less were discarded, along with their paired read. Exclusion of both reads was implemented after verifying that retention of unpaired reads did not significantly increase high quality alignment coverage. Quality control of the original and filtered FASTQ files was performed with FastQC (bioinformatics.babraham.ac.uk/projects/fastqc), deployed to verify the reduction in adapter content and the increase in base quality following the filtering stage. Removal of duplicates was performed at the pre-alignment stage with FastUniq. Duplicate pair-ends were removed by comparing sequences rather than post-aligned coordinates, allowing preservation of variant information.

Sequence alignment: Sequences were aligned to GRCh37/hg19 assembly of the human genome applying paired-reads Bowtie 2. Discordant pairs or constructed fragments larger than 1000 bp were discarded, thus improving mapping quality by allowing both reads to support mapping decisions. Default values (Bowtie 2 sensitive mode) were applied to end-to-end algorithm parameters, seed parameters, and bonus and penalty figures. Outputted SAM and BAM alignment files were examined using Picard CollectInsertSizeMetrics utility to verify correctness of final insert-size distribution (broadinstitute.github.io/picard. Version 1.119).

Variation calling: A BCF pileup file was generated from each BAM files using samtools mpileup function, set to consider bases of minimal Phred quality of 30 and minimal mapping quality of 30. Variant calling performed using bcftools, was initially set to output SNPs only to create SNP VCF files, according to the recommended setting for cancer. The VCF files were filtered by applying depth of coverage (DP) above 40 and statistical Quality (QUAL) above 10. DP filtering in this context refers to DP/INFO in the VCF file, which is a raw count of bases.

Variant post-processing: Post-processing of VCF SNPs included additional filtering, variant frequency calculation, mapping variants to probes and mapping variants to public databases, performed with a custom-written Python script. Additional depth coverage filtering of 20 was applied on the high-quality bases, which were selected by bcftools as appropriate for allelic counts. Frequency calculations were based on high-quality allelic depth (ratio of each allelic depth to sum of all allelic depths). SNPs were mapped to the following dbSNP and ClinVar databases: dbSNP/common version 20170710, dbSNP/All version 20170710 and clinvar_20170905.vcf. A match was determined when the position, reference and variant were all in agreement. In the analysis, de-novo variations (not in COMMON and not in ALL) which were detected in at least one sample (of eight) are referred to. For each targeted gene, the number of de-novo variations that were at a distance of +500 bp from its correlated sites were counted.

Regulatory CNVs: Non-coding CNVs were detected from WGS of 5Kbp sliding blocks in a 2Mbp region flanking gene TSSs, with a 50% overlap. Correlation of the total copy number TCN of each block with the gene expression level was assessed (at least six samples with available TCN data, Pearson and Spearman correlation). Correlation p values were adjusted for multiple-hypothesis testing using the Benjamini-Hochberg method.

### Genome Editing

Design and cloning of sgRNA: Guides to perturb SMO regulatory units were designed using the ChopChop, E-CRISP and CRISPOR softwares. 20-bp sgRNA sequences followed by the PAM ‘NGG’ for each unit, were identified and synthesized (see Table 1). For the SMO regulatory unit at chr7: 128,507,000-128,513,000 designated unit “A”, 4 guides were cloned into a backbone vector bearing Puromycin resistance (Addgene, 51133), using the Golden Gate assembly kit (NEB® Golden Gate Assembly Kit #E1601). Each guide sequence was cloned with its own U6 promoter and was followed by a sgRNA scaffold. For the regulatory unit at chr7: 129,384,500-129,389,500, designated unit “D”, two guides were cloned into the same backbone plasmid using the same method (FIG. 11).

Transfection/CRISPR-Cas9-mediated deletion: After validating the sgRNA sequences by Sanger sequencing, T98G or T98GdeltaSMO-D cells were co-transfected with a Cas9-bearing plasmid (Addgene, 48138) and either the plasmid bearing the guides targeting SMO A, the plasmid bearing the guides targeting SMO D, or the same plasmid harboring a non-targeting gRNA sequence (scramble), as a negative control. The molar ratio between the transfected guide plasmid and the Cas9 plasmid was 1:3, in favor of the plasmid not carrying the antibiotic resistance. 1.5-3*10∧5 cells/ml, >90% viable, were plated one day prior to transfection in a 6-well dish. On the transfection day, each well received 3 microliter Lipofectamine® 3000 Reagent, 5 microgram total plasmid DNA and 10 μl of Lipofectamine® 3000 Reagent (2:1 ratio). Puromycin (3 micrograms/microliter) was added to the cells one day after transfection. After 72 h, the antibiotic was washed, and the cells were left to expand. The cells were harvested 8-21d post-transfection and genomic DNA and RNA were immediately collected (Qiagen; DNeasy #69504 and RNeasy #74106, respectively).

Genotyping of mutant populations: Genomic DNA was subjected to genotyping PCR (primers listed in Table 2). Deletion or partial deletion was confirmed by gel electrophoresis or TapeStation, by Sanger sequencing and by illumina MiSeq sequencing (150 bp paired-end). Sanger sequencing was analyzed using BLAST and the sequence logo was generated using ggseqlogo R package. RNA extracted from populations of cells bearing such mutations were then checked for an effect on SMO transcription level, using qPCR (QuantStudio 3 cycler, Applied Biosystems, Thermo Fisher Scientific).

Single-cell dilution to obtain CRISPR-targeted cell clones: Puromycin-selected cells were isolated by trypsinization, counted and diluted to a concentration of 20 cells/100 microliters. Diluted cells (200 microliters) were then serially diluted, to ensure single-cell occupancy of rows 6-8 (eight dilution series). By calibrating the number of cells in the first row it was ensured that single cells could be isolated from the sixth to eighth rows onwards. Cells were incubated until the low-density wells were confluent enough to be transferred to 24-, 12- and finally to 6-well plates. Selected clones were tested for a stable DNA profile and for SMO transcription level by genotyping PCR (primers listed in Table 2), followed by gel electrophoresis or TapeStation and qPCR analysis, respectively.

RT-qPCR: Each isolated mRNA (500 ng) was transcribed to cDNA using the Verso cDNA Synthesis Kit (#AB-1453/A, Thermo Fisher Scientific) according to provided instructions, using the oligo dT primer. qPCR was performed using the Fast SYBR™ Green Master Mix (#AB-4385612, Thermo Fisher Scientific) and qPCR primers for SMO and reference genes HPRT and TBP (see Table 2), on a QuantStudio 3 cycler (Applied Biosystems, Thermo Fisher Scientific). The reaction was conducted in triplicates, and 20 ng of template were placed in each well. For each primer set, a no-template control (NTC) was also run, to check for possible contamination. QuantStudio Design & Analysis Software v1.4.3 (Applied Biosystems, Thermo Fisher Scientific) was used for analysis. All presented data were based on three or more biological replications of the genome editing experiments, each with three technical repeats of the DNA and RNA.

TABLE 1
Guide list

| | |
|---|---|
| A1 | ACCCTGCGCGCCGAGGTATC (SEQ ID NO: 6) |
| A2 | GCGACCTGGGAGCCGCCGCC (SEQ ID NO: 7) |
| A3 | ACCGCCGGTGCCGACCTTTG (SEQ ID NO: 8) |
| A4 | GCGTGGTAGTCCTTCTCCGG (SEQ ID NO: 9) |
| D1 | GTCCTGCTCTATCTTGTCGT (SEQ ID NO: 10) |
| D2 | CACATGTAGGTCTTTCTGAC (SEQ ID NO: 11) |
| N1 | CCGGCTCTGGGACTTACACCAATG (SEQ ID NO: 12) |
| N2 | CCGGACGGTGGATCTTCTTTAGTT (SEQ ID NO: 13) |
| N3 | CCGGTCCACCTTTTTGTTTCCTCT (SEQ ID NO: 14) |
| N4 | CCGGAAGATGGATGTCCCAGCACC (SEQ ID NO: 15) |

TABLE 2
Primer list

| | | |
|---|---|---|
| Genotyping SMO A (F) | 1066F | GCAGTGCGCTCACTTCAAA (SEQ ID NO: 16) |
| Genotyping SMO A (R) | 1066R | CTCCTGGGGCGAGATCAAAG (SEQ ID NO: 17) |
| Genotyping SMO D (F) | 1069F | CATGGTCCCGGTTCCCATTTGG (SEQ ID NO: 18) |
| Genotyping SMO D (R) | 955R | GCCCTCCACAGACCAAACAGC (SEQ ID NO: 19) |
| Genotyping SMO NULL (F) | 1120F | GCTCAGTCTCAGTGTGGGAG (SEQ ID NO: 20) |
| Genotyping SMO NULL (R) | 1120R | GGCGTTTCCACAAGAGATGAGC (SEQ ID NO: 21) |
| qPCR SMO F | 950F | TGCTCATCGTGGGAGGCTACTT (SEQ ID NO: 22) |
| qPCR SMO R | 950R | ATCTTGCTGGCAGCCTTCTCAC (SEQ ID NO: 23) |
| qPCR HPRT F | 442F | TGACACTGGCAAAACAATGCA (SEQ ID NO: 24) |
| qPCR HPRT R | 442R | GGTCCTTTTCACCAGCAAGCT (SEQ ID NO: 25) |
| qPCR TBP F | 850F | TGCACAGGAGCCAAGAGTGAA (SEQ ID NO: 26) |
| qPCR TBP R | 850R | CACATCACAGCTCCCCACCA (SEQ ID NO: 27) |

### Statistics and Data Visualization

All analyses were performed using both public and custom scripts written in R (R-project.org) and MATLAB (The Mathworks, Inc.). Plots were generated using plotting functionalities in base R and using ggplot2 package (ggplot2.tidyverse.org) and corrplot package (github.com/taiyun/corrplot). Sequence logos were generated using the ggseqlogo package. Heatmaps were produced using the ComplexHeatmap package. Lasso regression was performed using the default parameters of gmlnet package.

### Example 1: Integrative Genetic-Epigenetic Maps of Cis-Regulatory Domains

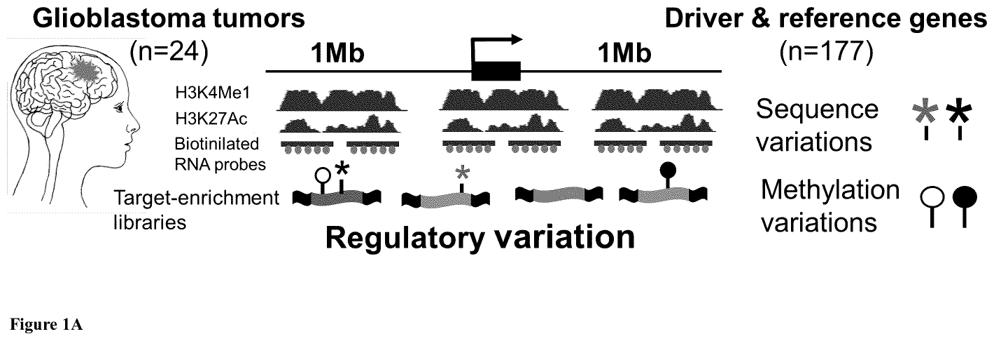

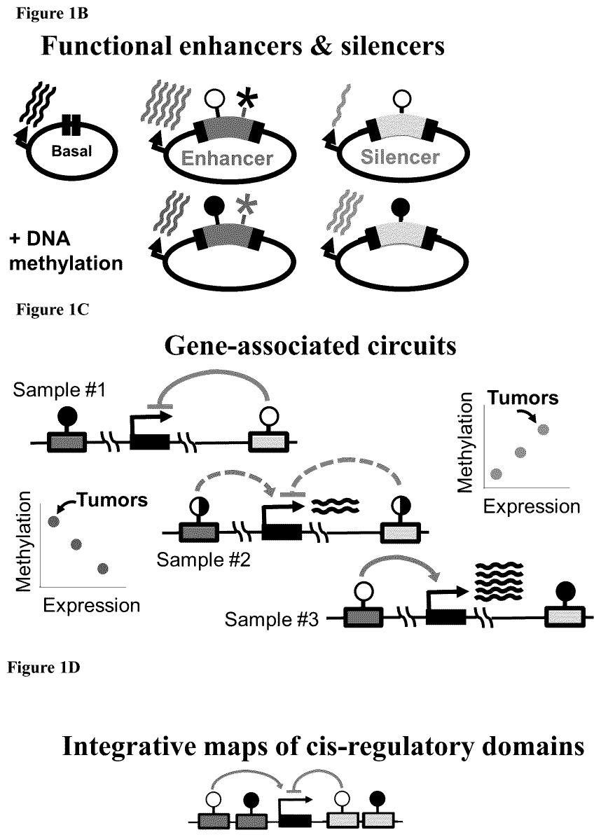

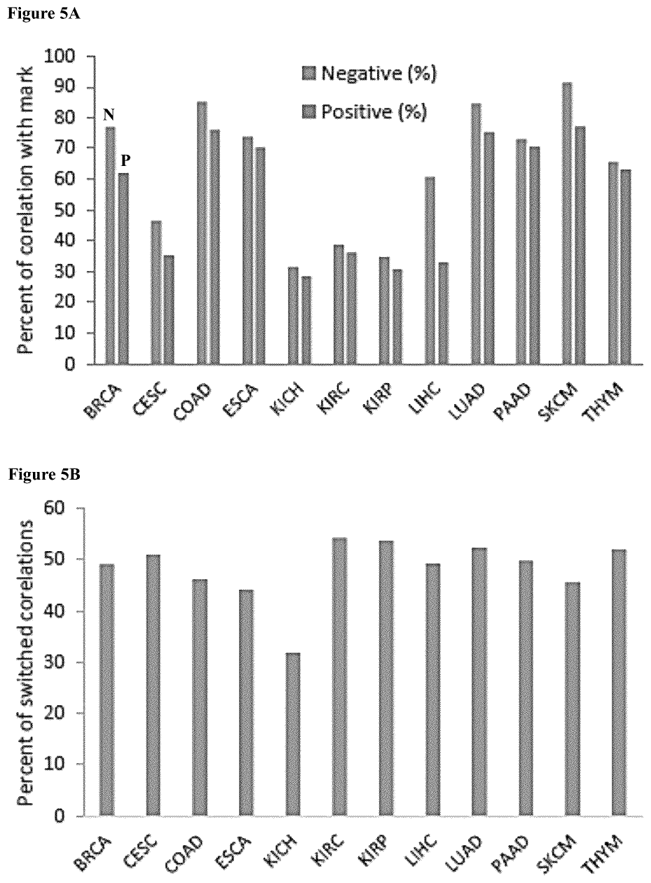

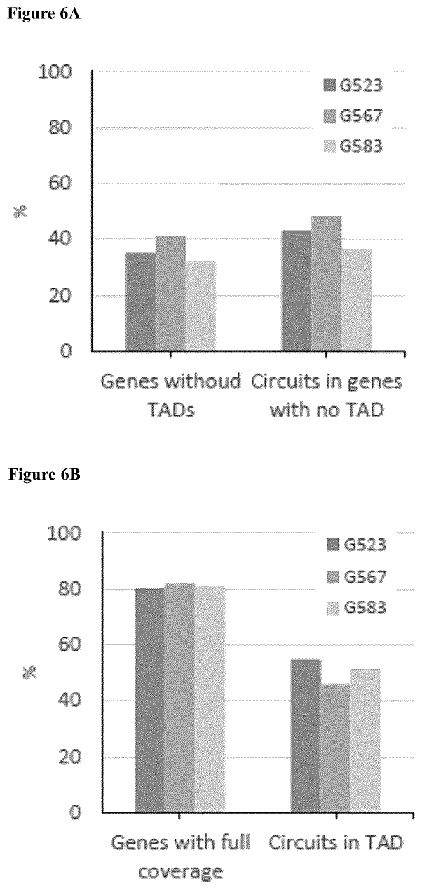

A strategy for methylation-centered interrogations of functional gene-associated regulatory elements was developed. While the method is applicable to many genes and diseases, the focus was on 125 pan-cancer and/or glioblastoma (GBM) driver genes, and 52 reference genes (Table 3). To focus on regulatory sites that may alternate their mode of action across tumors, initially the regulatory inputs provided by Histone 3 mono-methylated Lysine 4 (H3K4me1)-marked sites among various types of cancer were evaluated. Clearly, H3K4me1 sites showed similar frequencies of positive and negative associations between methylation and expression levels (FIG. 5A). Moreover, many of these sites switch between positive and negative effects on expression of the given genes, across cancers (FIG. 5B). Based on these observations, loci that carry H3K4me1 marks, and also the activity marker H3K27ac in some (but not all) of subjected glioblastoma tumors were targeted (see Materials and Methods). An analysis of normal and cancerous brains showed relative enrichment of DNase hypersensitivity signals within the targeted chromatin regions, thus confirming their regulatory potential. Many of the target genes were not firmly assigned to particular topologically-associated domains (TADs) (FIG. 6A-B). Therefore, it was chosen that all putative cis-acting regulatory elements were allocate within two million-base pair (Mbp) windows around the target gene promoters, thus ensuring unbiased evaluations of gene-associated sites within equivalent genomic spans. RNA probes (n=38,050, 120 bp each) were designed for all CpG methylation sites (n=140,494) within these chromatin blocks (SEQ ID NO: 28-38077). By targeting the RNA probes to GBM tumors across patients with age, gender and GBM-subtype ranges characteristic of this disease, libraries of captured DNA segments were obtained representing the spectrum of sequence and methylation variations of the tumors. These libraries served as input material for parallel analyses of the regulatory function and the gene-association status of the targeted loci (FIG. 1A-D, and 7).

TABLE 3
Drive and reference genes

| | | | | | | Non- | | |
|---|---|---|---|---|---|---|---|---|
| | | | | | | driver | Non- | Cancer |
| | | | | | | candidate | driver | type- |
| Gene | | | | | Driver | GBM | variable | specific |
| Symbol | Entrez ID | Chrom. | txStart | txEnd | gene | gene | gene | gene |
| ABL1 | 25 | CHR9 | 133589267 | 133763062 | Yes | 0 | 0 | 1 |
| CASP8 | 841 | CHR2 | 202098165 | 202152434 | Yes | 0 | 0 | 1 |
| DNMT1 | 1786 | CHR19 | 10244021 | 10305755 | Yes | 0 | 0 | 1 |
| EGFR | 1956 | CHR7 | 55086724 | 55275031 | Yes | 0 | 0 | 1 |
| FGFR3 | 2261 | CHR4 | 1795038 | 1810599 | Yes | 0 | 0 | 1 |
| ACVR1B | 91 | CHR12 | 52345450 | 52390863 | Yes | 0 | 0 | 0 |
| AKT1 | 207 | CHR14 | 105235686 | 105262080 | Yes | 0 | 0 | 0 |
| ALK | 238 | CHR2 | 29415639 | 30144477 | Yes | 0 | 0 | 0 |
| APC | 324 | CHR5 | 112043201 | 112181936 | Yes | 0 | 0 | 0 |
| AR | 367 | CHRX | 66763873 | 66950461 | Yes | 0 | 0 | 0 |
| ARID1A | 8289 | CHR1 | 27022521 | 27108601 | Yes | 0 | 0 | 0 |
| ARID1B | 57492 | CHR6 | 157099063 | 157531913 | Yes | 0 | 0 | 0 |
| ARID2 | 196528 | CHR12 | 46123619 | 46301819 | Yes | 0 | 0 | 0 |
| ASXL1 | 171023 | CHR20 | 30946146 | 31027122 | Yes | 0 | 0 | 0 |
| ATM | 472 | CHR11 | 108093558 | 108239826 | Yes | 0 | 0 | 0 |
| ATRX | 546 | CHRX | 76760355 | 77041755 | Yes | 0 | 0 | 0 |
| AXIN1 | 8312 | CHR16 | 337439 | 402676 | Yes | 0 | 0 | 0 |
| B2M | 567 | CHR15 | 45003684 | 45010357 | Yes | 0 | 0 | 0 |
| BAP1 | 8314 | CHR3 | 52435019 | 52444121 | Yes | 0 | 0 | 0 |
| BCL2 | 596 | CHR18 | 60790578 | 60986613 | Yes | 0 | 0 | 0 |
| BCOR | 54880 | CHRX | 39910498 | 40036582 | Yes | 0 | 0 | 0 |
| BRAF | 673 | CHR7 | 140433812 | 140624564 | Yes | 0 | 0 | 0 |
| BRCA1 | 672 | CHR17 | 41196311 | 41277500 | Yes | 0 | 0 | 0 |
| BRCA2 | 675 | CHR13 | 32889616 | 32973809 | Yes | 0 | 0 | 0 |
| CARD11 | 84433 | CHR7 | 2945709 | 3083579 | Yes | 0 | 0 | 0 |
| CBL | 867 | CHR11 | 119076985 | 119178859 | Yes | 0 | 0 | 0 |
| CDC73 | 79577 | CHR1 | 193091087 | 193223942 | Yes | 0 | 0 | 0 |
| CDH1 | 999 | CHR16 | 68771194 | 68869444 | Yes | 0 | 0 | 0 |
| CDKN2A | 1029 | CHR9 | 21967750 | 21994490 | Yes | 0 | 0 | 0 |
| CDKN2C | 1031 | CHR1 | 51434366 | 51440309 | Yes | 0 | 0 | 0 |
| CEBPA | 1050 | CHR19 | 33790839 | 33793470 | Yes | 0 | 0 | 0 |
| CHEK2 | 11200 | CHR22 | 29083730 | 29137822 | Yes | 0 | 0 | 0 |
| CIC | 23152 | CHR19 | 42772688 | 42799948 | Yes | 0 | 0 | 0 |
| CREBBP | 1387 | CHR16 | 3775055 | 3930121 | Yes | 0 | 0 | 0 |
| CSF1R | 1436 | CHR5 | 149432853 | 149492935 | Yes | 0 | 0 | 0 |
| CTNNB1 | 1499 | CHR3 | 41240941 | 41281939 | Yes | 0 | 0 | 0 |
| CYLD | 1540 | CHR16 | 50775960 | 50835846 | Yes | 0 | 0 | 0 |
| DAXX | 1616 | CHR6 | 33286334 | 33290793 | Yes | 0 | 0 | 0 |
| DNMT3A | 1788 | CHR2 | 25455829 | 25565459 | Yes | 0 | 0 | 0 |
| EP300 | 2033 | CHR22 | 41488613 | 41576081 | Yes | 0 | 0 | 0 |
| ERBB2 | 2064 | CHR17 | 37844336 | 37884915 | Yes | 0 | 0 | 0 |
| EZH2 | 2146 | CHR7 | 148504463 | 148581441 | Yes | 0 | 0 | 0 |
| FBXW7 | 55294 | CHR4 | 153242409 | 153456393 | Yes | 0 | 0 | 0 |
| FGFR2 | 2263 | CHR10 | 123237843 | 123357972 | Yes | 0 | 0 | 0 |
| FLT3 | 2322 | CHR13 | 28577410 | 28674729 | Yes | 0 | 0 | 0 |
| FOXL2 | 668 | CHR3 | 138663065 | 138665982 | Yes | 0 | 0 | 0 |
| FUBP1 | 8880 | CHR1 | 78412166 | 78444889 | Yes | 0 | 0 | 0 |
| GATA1 | 2623 | CHRX | 48644981 | 48652717 | Yes | 0 | 0 | 0 |
| GATA2 | 2624 | CHR3 | 128198264 | 128212030 | Yes | 0 | 0 | 0 |
| GATA3 | 2625 | CHR10 | 8096666 | 8117164 | Yes | 0 | 0 | 0 |
| GNA11 | 2767 | CHR19 | 3094407 | 3124000 | Yes | 0 | 0 | 0 |
| GNAQ | 2776 | CHR9 | 80331189 | 80646365 | Yes | 0 | 0 | 0 |
| GNAS | 2778 | CHR20 | 57414794 | 57486250 | Yes | 0 | 0 | 0 |
| H3F3A | 3020 | CHR1 | 226250407 | 226259703 | Yes | 0 | 0 | 0 |
| HNF1A | 6927 | CHR12 | 121416548 | 121440314 | Yes | 0 | 0 | 0 |
| HRAS | 3265 | CHR11 | 532241 | 535550 | Yes | 0 | 0 | 0 |
| IDH1 | 3417 | CHR2 | 209100950 | 209119867 | Yes | 0 | 0 | 0 |
| IDH2 | 3418 | CHR15 | 90627210 | 90645786 | Yes | 0 | 0 | 0 |
| JAK1 | 3716 | CHR1 | 65298905 | 65432187 | Yes | 0 | 0 | 0 |
| JAK2 | 3717 | CHR9 | 4985244 | 5128183 | Yes | 0 | 0 | 0 |
| JAK3 | 3718 | CHR19 | 17935592 | 17958841 | Yes | 0 | 0 | 0 |
| KDM5C | 8242 | CHRX | 53220502 | 53254604 | Yes | 0 | 0 | 0 |
| KDM6A | 7403 | CHRX | 44732420 | 44971857 | Yes | 0 | 0 | 0 |
| KIT | 3815 | CHR4 | 55524094 | 55606881 | Yes | 0 | 0 | 0 |
| KLF4 | 9314 | CHR9 | 110247132 | 110252047 | Yes | 0 | 0 | 0 |
| KMT2C | 58508 | CHR7 | 151832009 | 152133090 | Yes | 0 | 0 | 0 |
| KMT2D | 8085 | CHR12 | 49412757 | 49449107 | Yes | 0 | 0 | 0 |
| KRAS | 3845 | CHR12 | 25357722 | 25403865 | Yes | 0 | 0 | 0 |
| MAP2K1 | 5604 | CHR15 | 66679210 | 66783882 | Yes | 0 | 0 | 0 |
| MAP3K1 | 4214 | CHR5 | 56110899 | 56191978 | Yes | 0 | 0 | 0 |
| MED12 | 9968 | CHRX | 70338405 | 70362304 | Yes | 0 | 0 | 0 |
| MEN1 | 4221 | CHR11 | 64570985 | 64578766 | Yes | 0 | 0 | 0 |
| MET | 4233 | CHR7 | 116312458 | 116438440 | Yes | 0 | 0 | 0 |
| MLH1 | 4292 | CHR3 | 37034840 | 37092337 | Yes | 0 | 0 | 0 |
| MPL | 4352 | CHR1 | 43803474 | 43820135 | Yes | 0 | 0 | 0 |
| MSH2 | 4436 | CHR2 | 47630205 | 47710367 | Yes | 0 | 0 | 0 |
| MSH6 | 2956 | CHR2 | 48010220 | 48034092 | Yes | 0 | 0 | 0 |
| MYD88 | 4615 | CHR3 | 38179968 | 38184512 | Yes | 0 | 0 | 0 |
| NCOR1 | 9611 | CHR17 | 15933407 | 16118874 | Yes | 0 | 0 | 0 |
| NF1 | 4763 | CHR17 | 29421944 | 29704695 | Yes | 0 | 0 | 0 |
| NF2 | 4771 | CHR22 | 29999544 | 30094589 | Yes | 0 | 0 | 0 |
| NFE2L2 | 4780 | CHR2 | 178095030 | 178129859 | Yes | 0 | 0 | 0 |
| NOTCH1 | 4851 | CHR9 | 139388895 | 139440238 | Yes | 0 | 0 | 0 |
| NOTCH2 | 4853 | CHR1 | 120454175 | 120612317 | Yes | 0 | 0 | 0 |
| NPM1 | 4869 | CHR5 | 170814707 | 170837888 | Yes | 0 | 0 | 0 |
| NRAS | 4893 | CHR1 | 115247084 | 115259515 | Yes | 0 | 0 | 0 |
| PAX5 | 5079 | CHR9 | 36833271 | 37034476 | Yes | 0 | 0 | 0 |
| PBRM1 | 55193 | CHR3 | 52579367 | 52719866 | Yes | 0 | 0 | 0 |
| PDGFRA | 5156 | CHR4 | 55095263 | 55164412 | Yes | 0 | 0 | 0 |
| PHF6 | 84295 | CHRX | 133507341 | 133562822 | Yes | 0 | 0 | 0 |
| PIK3CA | 5290 | CHR3 | 178866310 | 178952497 | Yes | 0 | 0 | 0 |
| PIK3R1 | 5295 | CHR5 | 67511583 | 67597649 | Yes | 0 | 0 | 0 |
| PPP2R1A | 5518 | CHR19 | 52693054 | 52729678 | Yes | 0 | 0 | 0 |
| PRDM1 | 639 | CHR6 | 106534194 | 106557814 | Yes | 0 | 0 | 0 |
| PTCH1 | 5727 | CHR9 | 98205263 | 98279247 | Yes | 0 | 0 | 0 |
| PTEN | 5728 | CHR10 | 89623194 | 89731687 | Yes | 0 | 0 | 0 |
| PTPN11 | 5781 | CHR12 | 112856535 | 112947717 | Yes | 0 | 0 | 0 |
| RB1 | 5925 | CHR13 | 48877882 | 49056026 | Yes | 0 | 0 | 0 |
| RET | 5979 | CHR10 | 43572516 | 43625797 | Yes | 0 | 0 | 0 |
| RNF43 | 54894 | CHR17 | 56429860 | 56494943 | Yes | 0 | 0 | 0 |
| RPL5 | 6125 | CHR1 | 93297593 | 93307481 | Yes | 0 | 0 | 0 |
| RUNX1 | 861; | CHR21 | 36160097 | 36421595 | Yes | 0 | 0 | 0 |
| | 100506403 | | | | | | | |
| SETBP1 | 26040 | CHR18 | 42260137 | 42648475 | Yes | 0 | 0 | 0 |
| SETD2 | 29072 | CHR3 | 47057897 | 47205467 | Yes | 0 | 0 | 0 |
| SF3B1 | 23451 | CHR2 | 198256697 | 198299771 | Yes | 0 | 0 | 0 |
| SMAD2 | 4087 | CHR18 | 45359465 | 45457517 | Yes | 0 | 0 | 0 |
| SMAD4 | 4089 | CHR18 | 48556582 | 48611411 | Yes | 0 | 0 | 0 |
| SMARCA4 | 6597 | CHR19 | 11071597 | 11172958 | Yes | 0 | 0 | 0 |
| SMARCB1 | 6598 | CHR22 | 24129149 | 24176705 | Yes | 0 | 0 | 0 |
| SMO | 6608 | CHR7 | 128828712 | 128853385 | Yes | 0 | 0 | 0 |
| SOCS1 | 8651 | CHR16 | 11348273 | 11350039 | Yes | 0 | 0 | 0 |
| SOX9 | 6662 | CHR17 | 70117160 | 70122560 | Yes | 0 | 0 | 0 |
| SPOP | 8405 | CHR17 | 47676245 | 47755525 | Yes | 0 | 0 | 0 |
| SRSF2 | 6427 | CHR17 | 74730196 | 74733493 | Yes | 0 | 0 | 0 |
| STAG2 | 10735 | CHRX | 123094409 | 123236505 | Yes | 0 | 0 | 0 |
| STK11 | 6794 | CHR19 | 1205797 | 1228434 | Yes | 0 | 0 | 0 |
| TET2 | 54790 | CHR4 | 106067031 | 106200960 | Yes | 0 | 0 | 0 |
| TNFAIP3 | 7128 | CHR6 | 138188324 | 138204451 | Yes | 0 | 0 | 0 |
| TP53 | 7157 | CHR17 | 7571719 | 7590868 | Yes | 0 | 0 | 0 |
| TRAF7 | 84231 | CHR16 | 2205798 | 2228130 | Yes | 0 | 0 | 0 |
| TSC1 | 7248 | CHR9 | 135766734 | 135820020 | Yes | 0 | 0 | 0 |
| TSHR | 7253 | CHR14 | 81421868 | 81612646 | Yes | 0 | 0 | 0 |
| U2AF1 | 7307; | CHR21 | 44513065 | 44527688 | Yes | 0 | 0 | 0 |
| | 102724594 | | | | | | | |
| VHL | 7428 | CHR3 | 10183318 | 10195354 | Yes | 0 | 0 | 0 |
| WT1 | 7490 | CHR11 | 32409321 | 32457081 | Yes | 0 | 0 | 0 |
| DLL3 | 10683 | CHR19 | 39989556 | 39999121 | No | 1 | 0 | 1 |
| AKT2 | 208 | CHR19 | 40736223 | 40791302 | No | 0 | 0 | 1 |
| CASP5 | 838 | CHR11 | 104864966 | 104893895 | No | 0 | 0 | 1 |
| CHI3L1 | 1116 | CHR1 | 203148058 | 203155922 | No | 0 | 0 | 1 |
| ERBB3 | 2065 | CHR12 | 56473808 | 56497291 | No | 0 | 0 | 1 |
| FBXO3 | 26273 | CHR11 | 33762489 | 33796071 | No | 0 | 0 | 1 |
| GABRB2 | 2561 | CHR5 | 160715435 | 160975130 | No | 0 | 0 | 1 |
| MBP | 4155 | CHR18 | 74690788 | 74844774 | No | 0 | 0 | 1 |
| NES | 10763 | CHR1 | 156638555 | 156647189 | No | 0 | 0 | 1 |
| OLIG2 | 10215 | CHR21 | 34398215 | 34401503 | No | 0 | 0 | 1 |
| PDGFA | 5154 | CHR7 | 536896 | 559481 | No | 0 | 0 | 1 |
| RELB | 5971 | CHR19 | 45504706 | 45541456 | No | 0 | 0 | 1 |
| SNCG | 6623 | CHR10 | 88718287 | 88723017 | No | 0 | 0 | 1 |
| SOX2 | 6657 | CHR3 | 181429711 | 181432223 | No | 0 | 0 | 1 |
| TLR2 | 7097 | CHR4 | 154605440 | 154627242 | No | 0 | 0 | 1 |
| TLR4 | 7099 | CHR9 | 120466452 | 120479769 | No | 0 | 0 | 1 |
| TOP1 | 7150 | CHR20 | 39657461 | 39753126 | No | 0 | 0 | 1 |
| TRADD | 8717 | CHR16 | 67188088 | 67193812 | No | 0 | 0 | 1 |
| IGFBP6 | 3489 | CHR12 | 53491435 | 53496128 | No | 1 | 1 | 0 |
| AQP9 | 366 | CHR15 | 58430407 | 58478110 | No | 0 | 1 | 0 |
| BATF | 10538 | CHR14 | 75988783 | 76013334 | No | 0 | 1 | 0 |
| CD68 | 968 | CHR17 | 7482804 | 7485429 | No | 0 | 1 | 0 |
| DMRTA2 | 63950 | CHR1 | 50883222 | 50889119 | No | 0 | 1 | 0 |
| DSCAML1 | 57453 | CHR11 | 117298487 | 117667976 | No | 0 | 1 | 0 |
| EN1 | 2019 | CHR2 | 119599746 | 119605759 | No | 0 | 1 | 0 |
| FCGR2B | 2213 | CHR1 | 161632904 | 161648444 | No | 0 | 1 | 0 |
| FPR2 | 2358 | CHR19 | 52264452 | 52273779 | No | 0 | 1 | 0 |
| GLYATL2 | 219970 | CHR11 | 58601539 | 58611997 | No | 0 | 1 | 0 |
| HK3 | 3101 | CHR5 | 176307869 | 176326333 | No | 0 | 1 | 0 |
| IFI30 | 10437 | CHR19 | 18284589 | 18288934 | No | 0 | 1 | 0 |
| LGi3 | 203190 | CHR8 | 22004342 | 22014344 | No | 0 | 1 | 0 |
| LILRB2 | 10288 | CHR19 | 54777674 | 54785033 | No | 0 | 1 | 0 |
| LYVE1 | 10894 | CHR11 | 10579412 | 10590365 | No | 0 | 1 | 0 |
| SGCD | 6444 | CHR5 | 155753766 | 156194798 | No | 0 | 1 | 0 |
| SLC17A7 | 57030 | CHR19 | 49932654 | 49944808 | No | 0 | 1 | 0 |
| SOX10 | 6663 | CHR22 | 38368318 | 38380539 | No | 0 | 1 | 0 |
| SPHK1 | 8877 | CHR17 | 74380689 | 74383941 | No | 0 | 1 | 0 |
| VIPR2 | 7434 | CHR7 | 158820865 | 158937649 | No | 0 | 1 | 0 |
| ZIC2 | 7546 | CHR13 | 100634025 | 100639019 | No | 0 | 1 | 0 |
| ZNF676 | 163223 | CHR19 | 22361902 | 22379753 | No | 0 | 1 | 0 |
| ACSS3 | 79611 | CHR12 | 81471808 | 81649582 | No | 1 | 0 | 0 |
| ASXL3 | 80816 | CHR18 | 31158540 | 31327399 | No | 1 | 0 | 0 |
| BCAT1 | 586 | CHR12 | 24962957 | 25102393 | No | 1 | 0 | 0 |
| CA12 | 771 | CHR15 | 63615729 | 63674309 | No | 1 | 0 | 0 |
| CD163 | 9332 | CHR12 | 7623411 | 7656414 | No | 1 | 0 | 0 |
| CD177 | 57126 | CHR19 | 43857810 | 43867324 | No | 1 | 0 | 0 |
| FGF17 | 8822 | CHR8 | 21900263 | 21906319 | No | 1 | 0 | 0 |
| FGF9 | 2254 | CHR13 | 22245214 | 22278640 | No | 1 | 0 | 0 |
| GDF15 | 9518 | CHR19 | 18496967 | 18499986 | No | 1 | 0 | 0 |
| GRIA4 | 2893 | CHR11 | 105480799 | 105852819 | No | 1 | 0 | 0 |
| GRID2 | 2895 | CHR4 | 93225549 | 94695706 | No | 1 | 0 | 0 |
| LIF | 3976 | CHR22 | 30636435 | 30642840 | No | 1 | 0 | 0 |

### Example 2: Enhancers and Silencers are Co-Distributed Along Gene Domains

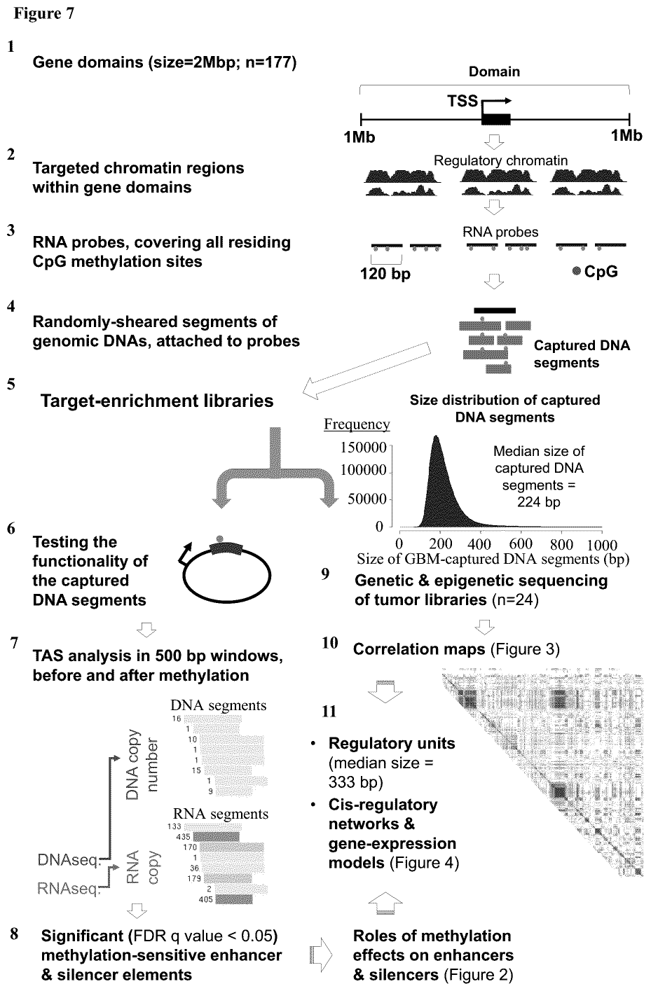

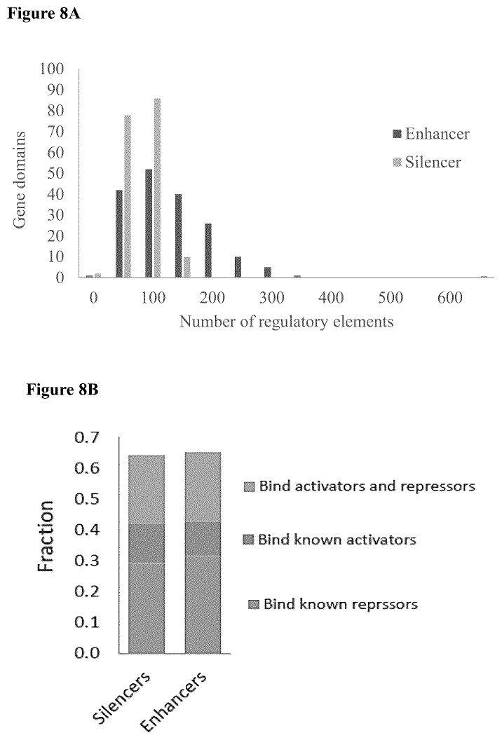

Functionality of the captured regulatory elements was examined in GBM cells, using a massively paralleled reporter assay adapted for detection of silencers and enhancers (see Materials and Methods). Transcriptional activity score (TAS) analysis revealed 26,152 significant (q<0.05) regulatory elements along the targeted gene domains, of them 9,204 silencers and 16,948 enhancers (FIG. 2A-C). An additional 16,030 targeted genomic elements showed no significant functions. Analysis of the chromatin around the annotated elements in a variety of other cell types, showed that the loci annotated as silencers or as enhancers in GBM cells shared the characteristics of open, TF-bound regulatory chromatin (FIG. 2D). In most (176 of 177) of the analyzed gene domains multiple (11-693) functional regulatory elements were observed. Of these domains, 175 contained both enhancers and silencers (FIG. 8A). It was concluded that regulatory elements are similarly distributed between enhancer and silencer functionalities across regulatory gene domains of GBM cells.

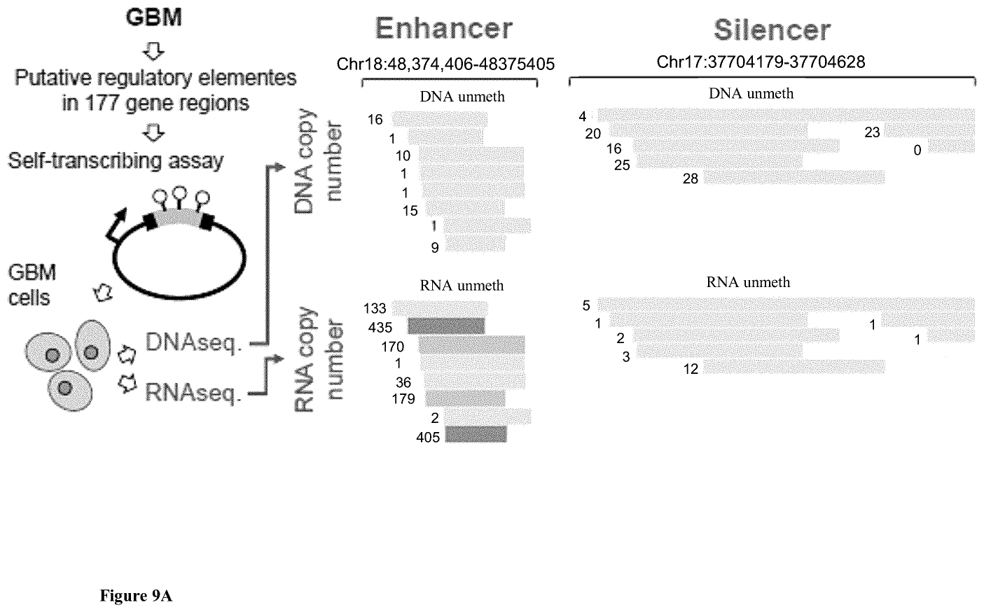

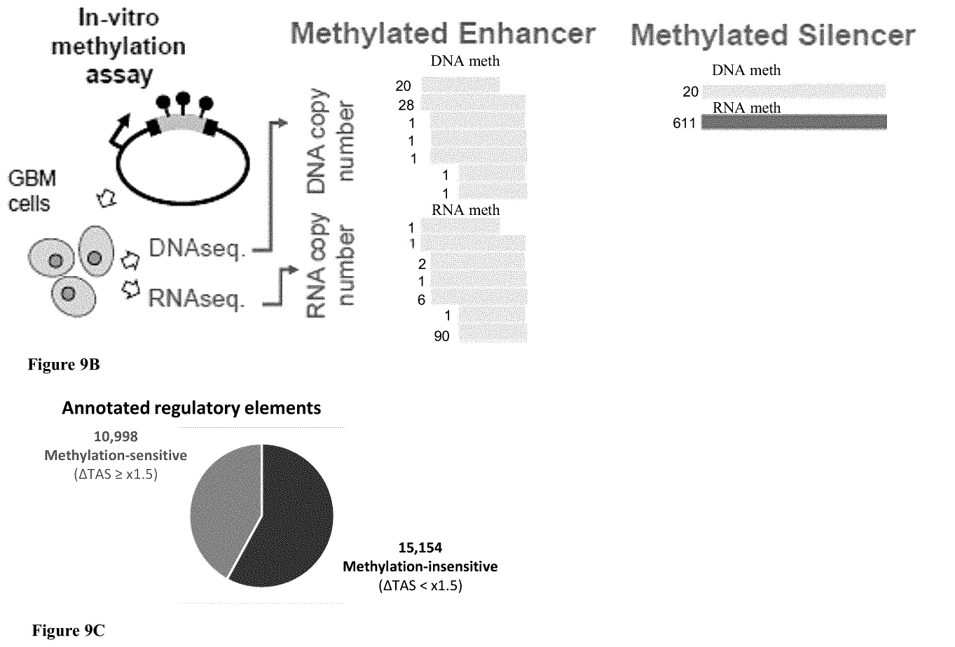

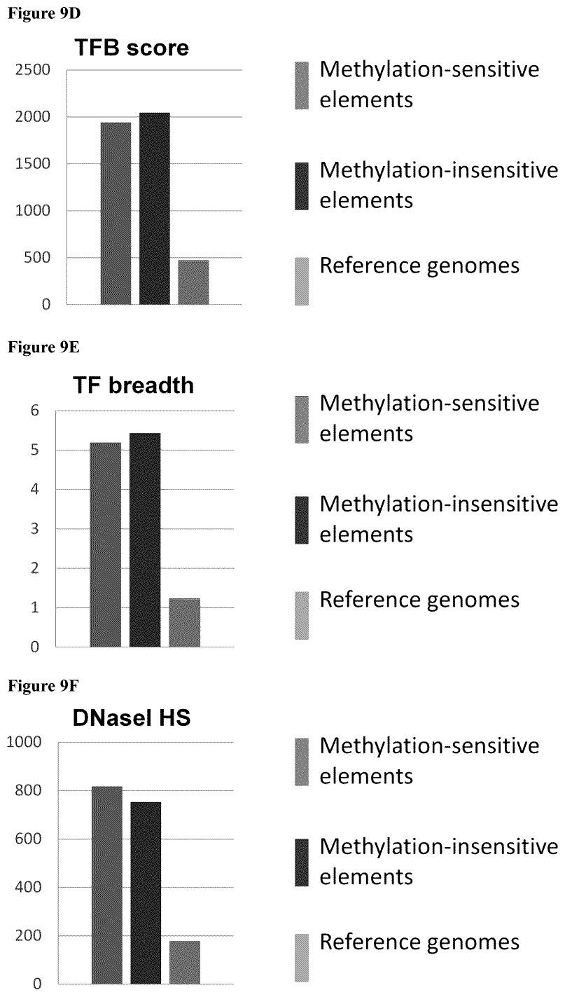

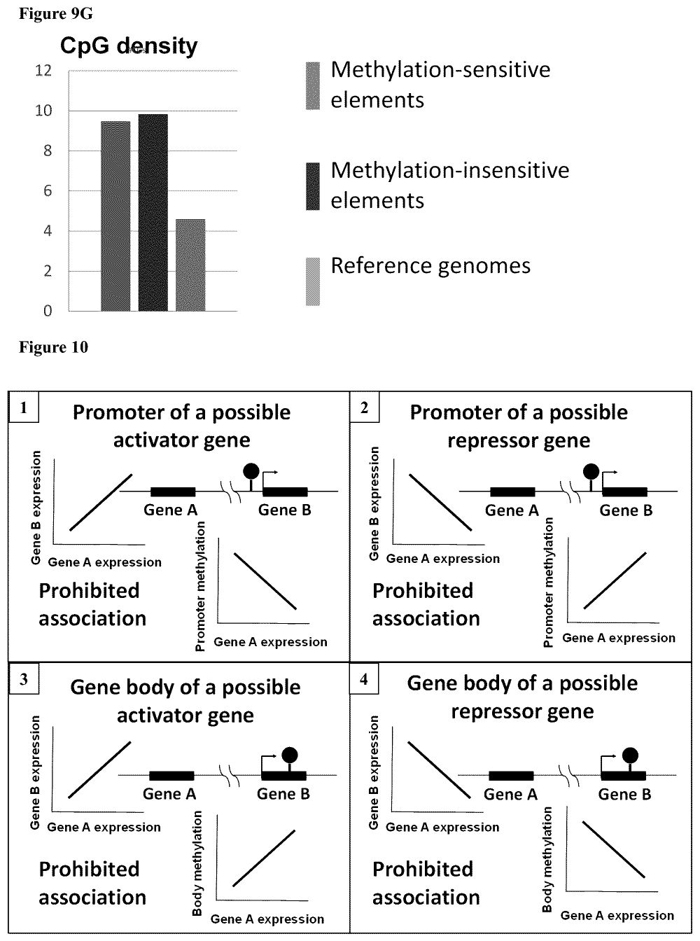

Example 3: DNA methylation induces enhancers and silencers to acquire new activity set points Across cell types, the analyzed regulatory elements bind both activators and repressors, regardless of their functional annotation in GBM (FIG. 8B), indicating the potential of these elements to mediate transcriptional enhancing or silencing, at different cellular conditions. It was explored whether DNA methylation directs their specific functioning in GBM. Instructive effects of methylation were examined by comparing the transcriptional outputs of reporter genes, driven by un-methylated or methylated cis-regulatory elements (FIG. 9A-B). Of the 26,152 annotated regulatory elements, 10,998 displayed ≥1.5-fold TAS differences between methylated and un-methylated states (FIG. 9C). The other 15,154 (57.9%) elements may be insensitive to methylation or affected below the detection threshold of the assay. Overall, DNA methylation generally reduced the activity levels of both enhancers and silencers (FIG. 2E). Of the methylation-sensitive silencers and enhancers, the majority (83.7%) reduced their original activities, so enhancers were shifted to lower enhancing activities upon methylation, and silencers were shifted to lower silencing effects, while 16.3% of the methylation-responding elements showed the opposite effect, i.e., increased regulatory activity upon DNA methylation (FIG. 2F). Interestingly, many elements were shifted to the opposing functionality (i.e., enhancers were turned to silencers, and vice versa), upon methylation (FIG. 2G). However, the effect of methylation was not restricted to complete switching between full enhancing and full silencing functionalities. Rather, it allowed silencers and enhancers to adopt new activity set points within ranges of enhancing to silencing effects, possibly by affecting the balance between bound activators and repressors. Interestingly, methylation-sensitive and -insensitive sites shared the characteristics of regulatory chromatin (FIG. 9D-G), suggesting that more specific differences underlie their distinguished responses to methylation (e.g., deferential binding of particular methylation-sensitive or methylation-resistant transcription factors). It was concluded that core regulatory sequences may be retuned on their operative scales, between enhancing and silencing inputs to the transcriptional machinery. DNA methylation is apparently required and sufficient to induce these effects in GBM cells.

### Example 4: Methylation Data Reveals the Cis-Regulatory Circuits of GBM Genes

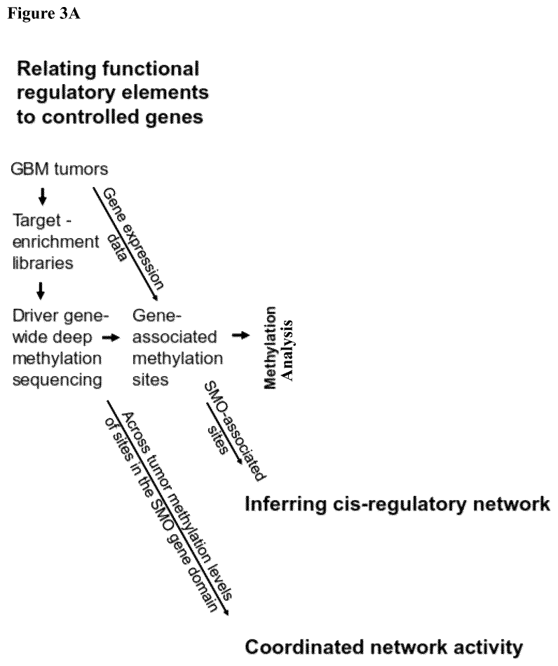

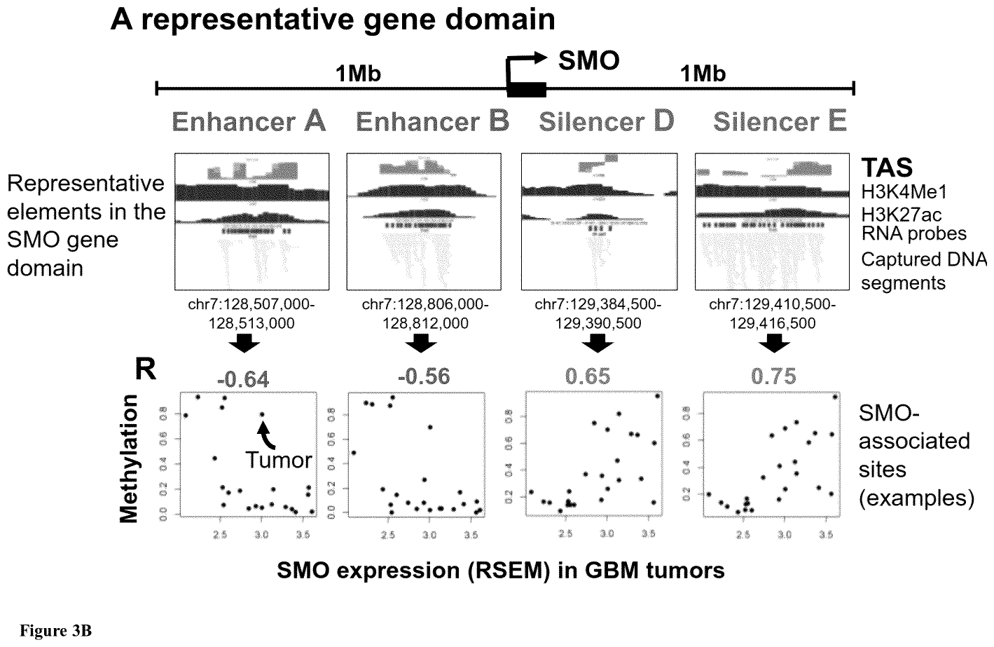

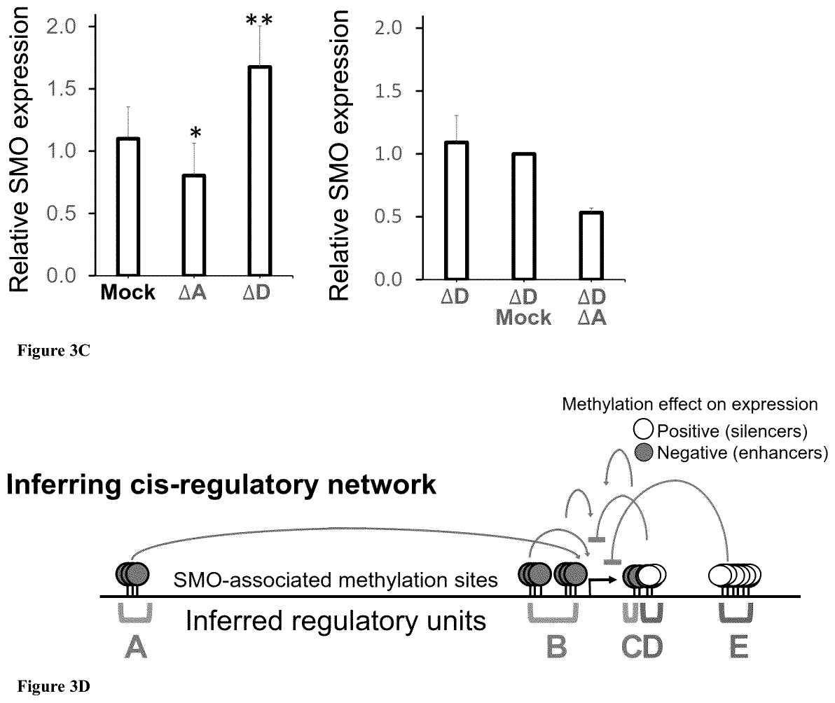

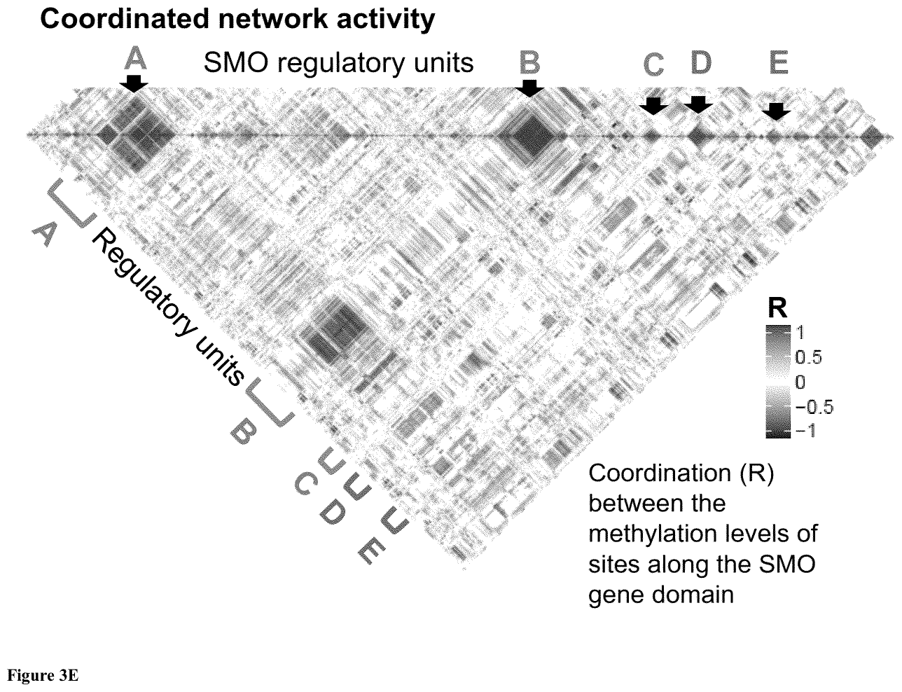

The above experiments detect the effect of methylation on core regulatory sequences at simplified genetic structure and under extreme, fully-methylated or fully-unmethylated conditions. These experiments revealed principal rules of methylation effect on enhancers and silencers (FIG. 2A-G). Since the conditions in actual GBM chromatin may be essentially different, next methylation-expression associations in intact GBM genomes was studied. Utilizing the same capturing libraries that were used for the functional assays, the correlation between the methylation levels of the captured sites and expression levels of the targeted genes were analyzed among 24 GBM samples (Table 3), applying the herein described method (FIG. 3A). To avoid possible indirect effects, gene-body and promoter sites (n=232), which may display methylation-expression associations due to secondary interactions, were excluded from the analysis (FIG. 10). The resultant significant correlations between methylation and expression levels across the GBM samples, revealed associations between certain regulatory sites and controlled genes (n=1,154; q<0.05; R2 >0.3, Table 4). These associations between regulatory sites and gene expression were termed the cis-regulatory circuits of the genes.

TABLE 4
Gene-associated regulatory units

| Gene | Unit ID | Chr. | Start | End | Span (bp) | Sites | Association |
|---|---|---|---|---|---|---|---|
| ABL1 | 1 | CHR9 | 132958046 | 132958649 | 603 | 4 | 1 |
| ABL1 | 2 | CHR9 | 132982490 | 132982643 | 153 | 2 | 1 |
| ABL1 | 3 | CHR9 | 133327005 | 133327821 | 816 | 2 | 1 |
| ABL1 | 4 | CHR9 | 133346631 | 133350389 | 3758 | 2 | 1 |
| AKT1 | 6 | CHR14 | 105636925 | 105637327 | 402 | 2 | 1 |
| AKT2 | 1 | CHR19 | 39993313 | 39994770 | 1457 | 13 | 1 |
| ASXL1 | 1 | CHR20 | 30429763 | 30431256 | 1493 | 2 | 1 |
| AXIN1 | 3 | CHR16 | 722369 | 724645 | 2276 | 2 | 1 |
| AXIN1 | 5 | CHR16 | 1088005 | 1088438 | 433 | 2 | 1 |
| AXIN1 | 7 | CHR16 | 1204532 | 1204751 | 219 | 2 | 1 |
| AXIN1 | 8 | CHR16 | 1381813 | 1382207 | 394 | 7 | 1 |
| BCOR | 3 | CHRX | 39343643 | 39344585 | 942 | 2 | −1 |
| BRCA2 | 1 | CHR13 | 33760688 | 33760693 | 5 | 2 | 1 |
| CA12 | 2 | CHR15 | 63254573 | 63255038 | 465 | 6 | 1 |
| CA12 | 4 | CHR15 | 64189128 | 64189197 | 69 | 3 | −1 |
| CDKN2A | 2 | CHR9 | 21576533 | 21576558 | 25 | 2 | −1 |
| CDKN2A | 3 | CHR9 | 21811216 | 21812891 | 1675 | 3 | −1 |
| CDKN2A | 4 | CHR9 | 22052216 | 22053197 | 981 | 4 | −1 |
| CDKN2A | 5 | CHR9 | 22079791 | 22080476 | 685 | 7 | −1 |
| CHEK2 | 1 | CHR22 | 29540086 | 29540489 | 403 | 4 | −1 |
| CHEK2 | 3 | CHR22 | 30091748 | 30091780 | 32 | 2 | −1 |
| CHEK2 | 4 | CHR22 | 30097763 | 30098062 | 299 | 2 | −1 |
| CHI3L1 | 1 | CHR1 | 203016451 | 203016480 | 29 | 3 | −1 |
| CHI3L1 | 2 | CHR1 | 203105193 | 203105354 | 161 | 2 | −1 |
| CHI3L1 | 3 | CHR1 | 203135787 | 203136651 | 864 | 5 | −1 |
| CHI3L1 | 6 | CHR1 | 203632398 | 203632511 | 113 | 2 | −1 |
| CHI3L1 | 7 | CHR1 | 204120492 | 204121836 | 1344 | 5 | −1 |
| CIC | 1 | CHR19 | 42569945 | 42570265 | 320 | 4 | 1 |
| CIC | 2 | CHR19 | 42656665 | 42656734 | 69 | 2 | 1 |
| CREBBP | 2 | CHR16 | 3238942 | 3239089 | 147 | 3 | 1 |
| DAXX | 4 | CHR6 | 33738809 | 33739114 | 305 | 2 | 1 |
| DAXX | 6 | CHR6 | 34032938 | 34033076 | 138 | 2 | −1 |
| DLL3 | 1 | CHR19 | 39360164 | 39361072 | 908 | 6 | 1 |
| DSCAML1 | 4 | CHR11 | 118186164 | 118186176 | 12 | 2 | 1 |
| EGFR | 1 | CHR7 | 54890403 | 54893102 | 2699 | 4 | 1 |
| EGFR | 2 | CHR7 | 54898637 | 54912505 | 13868 | 8 | 1 |
| EGFR | 3 | CHR7 | 55058032 | 55071675 | 13643 | 10 | 1 |
| EN1 | 1 | CHR2 | 119564489 | 119564855 | 366 | 12 | −1 |
| EN1 | 2 | CHR2 | 119599106 | 119599681 | 575 | 26 | −1 |
| ERBB2 | 2 | CHR17 | 37322124 | 37322310 | 186 | 4 | −1 |
| ERBB2 | 3 | CHR17 | 37752917 | 37757721 | 4804 | 3 | −1 |
| FGF17 | 1 | CHR8 | 21881722 | 21882709 | 987 | 7 | 1 |
| FGF17 | 3 | CHR8 | 22573255 | 22573260 | 5 | 2 | 1 |
| FGF17 | 5 | CHR8 | 22722594 | 22722935 | 341 | 3 | 1 |
| FGFR2 | 1 | CHR10 | 123196281 | 123196864 | 583 | 3 | −1 |
| FGFR3 | 1 | CHR4 | 816568 | 816608 | 40 | 3 | 1 |
| GATA1 | 1 | CHRX | 48326644 | 48326691 | 47 | 3 | 1 |
| GDF15 | 3 | CHR19 | 17790731 | 17791448 | 717 | 31 | −1 |
| GDF15 | 6 | CHR19 | 18210253 | 18210267 | 14 | 3 | −1 |
| GDF15 | 8 | CHR19 | 18342128 | 18342151 | 23 | 2 | −1 |
| GDF15 | 9 | CHR19 | 18412001 | 18412084 | 83 | 4 | −1 |
| GDF15 | 11 | CHR19 | 18906490 | 18906551 | 61 | 2 | −1 |
| GDF15 | 12 | CHR19 | 19221495 | 19221717 | 222 | 19 | −1 |
| GNA11 | 2 | CHR19 | 2722050 | 2722284 | 234 | 2 | 1 |
| GNAS | 1 | CHR20 | 56482663 | 56482712 | 49 | 2 | −1 |
| H3F3A | 4 | CHR1 | 226738547 | 226738917 | 370 | 3 | −1 |
| H3F3A | 5 | CHR1 | 227070288 | 227070967 | 679 | 2 | 1 |
| HK3 | 3 | CHR5 | 176829109 | 176829112 | 3 | 2 | 1 |
| HRAS | 1 | CHR11 | 416293 | 416732 | 439 | 2 | 1 |
| KDM5C | 2 | CHRX | 53034306 | 53034308 | 2 | 2 | 1 |
| KDM5C | 3 | CHRX | 53293024 | 53293044 | 20 | 2 | −1 |
| KLF4 | 1 | CHR9 | 109622425 | 109622770 | 345 | 9 | −1 |
| KMT2D | 3 | CHR12 | 49379024 | 49379309 | 285 | 2 | 1 |
| KMT2D | 4 | CHR12 | 49725964 | 49726144 | 180 | 2 | 1 |
| MBP | 1 | CHR18 | 74069561 | 74070447 | 886 | 2 | −1 |
| MBP | 2 | CHR18 | 74109928 | 74111699 | 1771 | 5 | −1 |
| MBP | 3 | CHR18 | 74155624 | 74155669 | 45 | 2 | −1 |
| MBP | 4 | CHR18 | 74170082 | 74171191 | 1109 | 6 | −1 |
| MBP | 6 | CHR18 | 74597515 | 74598613 | 1098 | 2 | −1 |
| MBP | 7 | CHR18 | 74685615 | 74685931 | 316 | 5 | −1 |
| MEN1 | 2 | CHR11 | 63769728 | 63769763 | 35 | 3 | 1 |
| MEN1 | 4 | CHR11 | 63850967 | 63851074 | 107 | 4 | 1 |
| MEN1 | 5 | CHR11 | 63904407 | 63904790 | 383 | 2 | 1 |
| MEN1 | 6 | CHR11 | 63916745 | 63917131 | 386 | 2 | 1 |
| MEN1 | 8 | CHR11 | 64120728 | 64121094 | 366 | 4 | 1 |
| MEN1 | 11 | CHR11 | 64306320 | 64306586 | 266 | 2 | 1 |
| MEN1 | 12 | CHR11 | 64403763 | 64403849 | 86 | 4 | 1 |
| MEN1 | 13 | CHR11 | 64611748 | 64614814 | 3066 | 2 | 1 |
| MLH1 | 2 | CHR3 | 37735694 | 37735713 | 19 | 2 | −1 |
| MYD88 | 3 | CHR3 | 38035569 | 38035661 | 92 | 2 | −1 |
| MYD88 | 4 | CHR3 | 38070605 | 38070746 | 141 | 12 | −1 |
| NES | 2 | CHR1 | 156594421 | 156595764 | 1343 | 12 | −1 |
| OLIG2 | 3 | CHR21 | 34207131 | 34207141 | 10 | 2 | −1 |
| OLIG2 | 4 | CHR21 | 34584855 | 34584896 | 41 | 2 | −1 |
| OLIG2 | 5 | CHR21 | 34610669 | 34610692 | 23 | 2 | 1 |
| PBRM1 | 7 | CHR3 | 53229676 | 53229827 | 151 | 2 | −1 |
| PDGFA | 1 | CHR7 | 204578 | 207549 | 2971 | 3 | −1 |
| PDGFA | 8 | CHR7 | 947378 | 949295 | 1917 | 17 | −1 |
| PDGFA | 9 | CHR7 | 997854 | 997865 | 11 | 2 | −1 |
| PDGFA | 10 | CHR7 | 1004681 | 1004748 | 67 | 2 | −1 |
| PDGFA | 12 | CHR7 | 1363132 | 1363196 | 64 | 3 | −1 |
| PDGFRA | 1 | CHR4 | 54179652 | 54180336 | 684 | 4 | −1 |
| PDGFRA | 4 | CHR4 | 55199007 | 55200197 | 1190 | 2 | −1 |
| PRDM1 | 3 | CHR6 | 107397800 | 107397809 | 9 | 2 | 1 |
| RELB | 2 | CHR19 | 46318566 | 46319244 | 678 | 5 | −1 |
| SGCD | 1 | CHR5 | 155108749 | 155109126 | 377 | 3 | −1 |
| SMAD2 | 2 | CHR18 | 45792196 | 45792274 | 78 | 3 | −1 |
| SMAD2 | 3 | CHR18 | 45837031 | 45837122 | 91 | 2 | −1 |
| SMAD2 | 5 | CHR18 | 46100503 | 46101057 | 554 | 5 | −1 |
| SMAD2 | 9 | CHR18 | 46258911 | 46259158 | 247 | 4 | −1 |
| SMAD2 | 10 | CHR18 | 46363532 | 46363764 | 232 | 2 | −1 |
| SMAD2 | 12 | CHR18 | 46446963 | 46448862 | 1899 | 2 | −1 |
| SMAD4 | 1 | CHR18 | 48179928 | 48181583 | 1655 | 2 | 1 |
| SMARCB1 | 1 | CHR22 | 23744655 | 23744863 | 208 | 5 | 1 |
| SMO | 1 | CHR7 | 128510136 | 128510159 | 23 | 4 | −1 |
| SMO | 2 | CHR7 | 128809090 | 128809500 | 410 | 9 | −1 |
| SMO | 3 | CHR7 | 129257134 | 129257460 | 326 | 2 | −1 |
| SMO | 4 | CHR7 | 129387084 | 129387304 | 220 | 2 | 1 |
| SMO | 5 | CHR7 | 129414098 | 129414746 | 648 | 12 | 1 |
| SOCS1 | 2 | CHR16 | 11327291 | 11327385 | 94 | 5 | −1 |
| SOX10 | 2 | CHR22 | 38846250 | 38849206 | 2956 | 9 | −1 |
| SOX10 | 3 | CHR22 | 39110893 | 39113018 | 2125 | 2 | −1 |
| SOX10 | 4 | CHR22 | 39125019 | 39126882 | 1863 | 8 | −1 |
| SOX10 | 6 | CHR22 | 39171695 | 39172892 | 1197 | 8 | −1 |
| SOX10 | 7 | CHR22 | 39225028 | 39226394 | 1366 | 3 | −1 |
| SOX9 | 2 | CHR17 | 70267379 | 70267410 | 31 | 2 | 1 |
| SOX9 | 3 | CHR17 | 70492916 | 70493349 | 433 | 2 | 1 |
| SOX9 | 5 | CHR17 | 70619853 | 70619923 | 70 | 3 | 1 |
| SRSF2 | 9 | CHR17 | 75653246 | 75653373 | 127 | 2 | −1 |
| STK11 | 1 | CHR19 | 583581 | 584951 | 1370 | 3 | 1 |
| STK11 | 2 | CHR19 | 591261 | 592783 | 1522 | 4 | 1 |
| STK11 | 4 | CHR19 | 676269 | 676739 | 470 | 3 | 1 |
| STK11 | 9 | CHR19 | 1285161 | 1285346 | 185 | 4 | 1 |
| STK11 | 11 | CHR19 | 1377927 | 1378043 | 116 | 5 | 1 |
| STK11 | 12 | CHR19 | 1396211 | 1399839 | 3628 | 5 | 1 |
| STK11 | 14 | CHR19 | 1667339 | 1667551 | 212 | 5 | 1 |
| TNFAIP3 | 2 | CHR6 | 138072762 | 138073229 | 467 | 2 | 1 |
| TNFAIP3 | 3 | CHR6 | 138833429 | 138833586 | 157 | 6 | 1 |
| TNFAIP3 | 4 | CHR6 | 138876257 | 138876305 | 48 | 3 | −1 |
| TNFAIP3 | 5 | CHR6 | 138975000 | 138976656 | 1656 | 5 | −1 |
| TRAF7 | 1 | CHR16 | 1381813 | 1382188 | 375 | 5 | 1 |
| TRAF7 | 2 | CHR16 | 1681574 | 1682480 | 906 | 2 | 1 |
| TRAF7 | 3 | CHR16 | 2075970 | 2077768 | 1798 | 2 | 1 |
| TRAF7 | 4 | CHR16 | 2106729 | 2106989 | 260 | 2 | 1 |
| VHL | 4 | CHR3 | 10545002 | 10545134 | 132 | 3 | −1 |
| VIPR2 | 5 | CHR7 | 158710580 | 158711458 | 878 | 6 | −1 |
| ZIC2 | 1 | CHR13 | 100619840 | 100620283 | 443 | 10 | −1 |
| ZIC2 | 2 | CHR13 | 100640027 | 100640092 | 65 | 9 | −1 |

Example 5: genomic editing experiments verify regulatory inputs in GBM chromatin The experimentally-identified regulatory elements were compared with the cis-regulatory circuits of GBM tumors. Merging of association and functional data revealed alignment of functional enhancers with negatively-associated sites, and of functional silencers with positive associations (FIG. 3B, 11). Genomic manipulation experiments were performed to verify particular predictions of the functional gene-association annotations. The Smoothened, Frizzled Class Receptor (SMO) driver-gene, for example, was abnormally expressed in 23 of the 24 tumors. Three functional enhancers and two functional silencers, consisting of 29 associated methylation sites, were found in the gene domain (Table 4). Indeed, removing a functional, SMO-associated enhancer from the genome of GBM cells reduced SMO expression relative to mock-treated cells, whereas deletion of a silencer unit increased its expression. Moreover, deletion of the enhancer unit has similar effect on the wild-type and silencer-deletion backgrounds (30-50% reduction relative to the background expression levels), suggesting that the enhancer and the silencer units provide additive inputs to the transcriptional machinery (FIG. 3C).

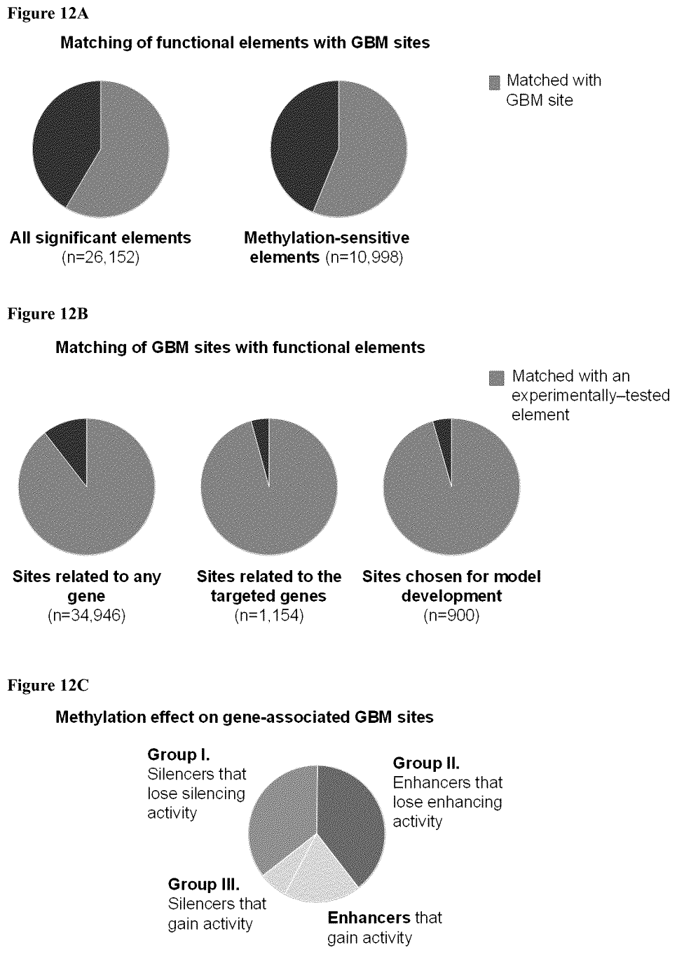

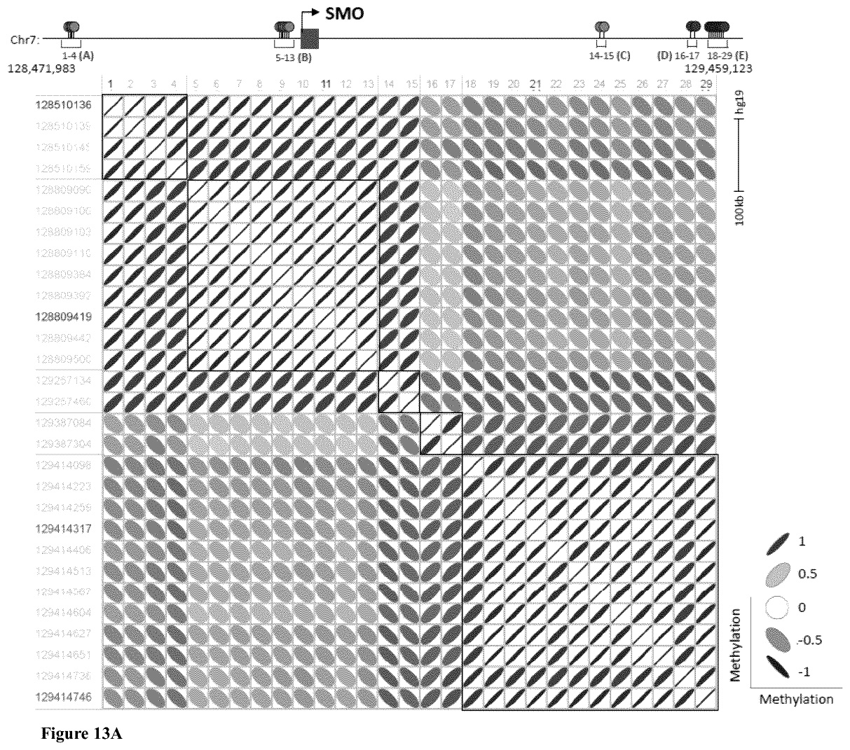

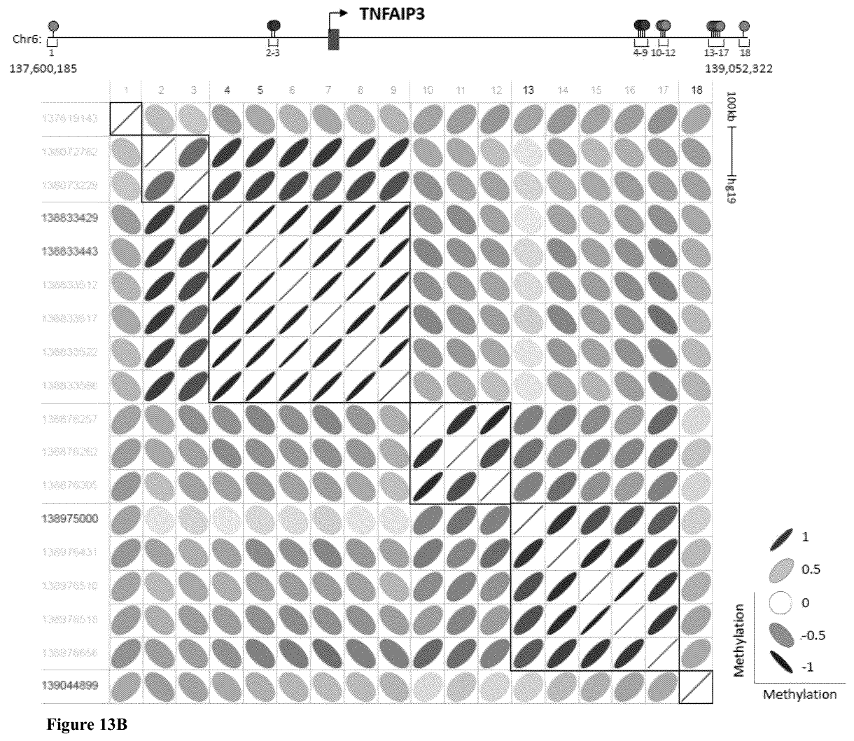

Overall, of the 26, 152 uncovered functional elements, 15,304 (58.5%) were matched with a GBM-associated site, located up to 500 bp from the element (FIG. 12A). The non-matching elements may be regulatory elements which are not functional in GBM cells, or due to the technical noise of the assays. To discern between the possibilities, the matching between GBM sites and functional elements was analyzed. Indeed, 95.7% of the 1,154 gene-associated methylation sites matched with a nearby element found by the experimental assay (FIG. 12B), suggesting that actual GBM-related methylation sites were effectively detected by the experimental assay. Moreover, TAS analyses of the actual gene-associated sites reveled patterns of methylation effects (FIG. 12C), similar to the patterns learned from TAS analysis of the experimentally-defined elements (FIG. 2F). It was concluded that the general rules of methylation effect on gene transcription, which were learned in the experimental assay, may be applied to bona fide GBM tumors.

### Example 6: Deep Methylation Analysis Reveals the Size and Organization of Cis-Regulatory Units

To explore the organization and function of the uncovered GBM circuits, the major groups (groups I and II in FIG. 2F and FIG. 12C) of enhancers and silencers were focused on. Hence, sites that, according to the reporter assays, may not belong to these classes were filtered out. The filter excludes 22% (254 of 1,154) circuits of the targeted genes. Of the remaining 900 regulatory circuits of 109 genes, 42% denoted positive relationships with expression, and 58% negative (Table 4). Most (78%) of the genes had multiple (2-68) circuits, averaging 8.3 (3.5 positive, 4.8 negative) circuits per gene (Table 5). This wide-coverage, high-resolution mapping of gene-associated sites provides a unique opportunity to detect the size and organization of actual regulatory units, embedded within large bodies of regulatory chromatin. It was found that gene-associated sites tend to form defined clusters, spanning tens to thousands (average 834, median 333) bps. Each of these clusters contained up to 31 associated sites, which mediate homogenous (positive or negative) input to the transcription of a particular gene. Since each CpG site was distinctly analyzed, these clusters are true learned features of the genome. Hence, gene regulatory domains contain sets of defined, gene-specific, enhancer and silencer units. They were termed gene-regulatory units.

TABLE 5
Methylation-based tumor profiling models

| | | | | | | | | Signif. | | | | |
|---|---|---|---|---|---|---|---|---|---|---|---|---|
| | | Asso. | Associations | | Best | Best | Possible | multi-site | Best | | Neg. | Pos. |
| Driver | Gene | sites | Neg. | Pos. | Neg. R | Pos. R | Combos | models | R | P-val. | sites | sites |
| Yes | ABL1 | 15 | 1 | 14 | −0.61 | 0.70 | 1925 | 1920 | 0.91 | 0.00038 | 1 | 3 |
| Yes | ACVR1B | 2 | 0 | 2 | 0.60 | 0.79 | 1 | 1 | 0.89 | 6.80E−05 | 0 | 2 |
| Yes | AKT1 | 8 | 0 | 8 | 0.55 | 0.63 | 132 | 12 | 0.76 | 0.00013 | 0 | 3 |
| Yes | BCOR | 5 | 4 | 1 | −0.65 | 0.58 | 25 | 5 | 0.73 | 0.00197 | 2 | 0 |
| Yes | BRCA1 | 3 | 2 | 1 | −0.69 | 0.57 | 2 | 2 | 0.74 | 0.0113 | 1 | 1 |
| Yes | CHEK2 | 9 | 9 | 0 | −0.72 | −0.59 | 246 | 245 | 0.93 | 0.00027 | 3 | 0 |
| Yes | CREBBP | 5 | 0 | 5 | 0.58 | 0.78 | 25 | 25 | 0.85 | 1.72E−05 | 0 | 3 |
| Yes | CTNNB1 | 2 | 2 | 0 | −0.68 | −0.64 | 1 | 1 | 0.71 | 0.00028 | 2 | 0 |
| Yes | DAXX | 12 | 5 | 7 | −0.73 | 0.69 | 781 | 781 | 0.87 | 1.26E−05 | 2 | 2 |
| Yes | DNMT3A | 2 | 2 | 0 | −0.74 | −0.66 | 1 | 1 | 0.74 | 8.05E−05 | 2 | 0 |
| Yes | FBXW7 | 2 | 2 | 0 | −0.62 | −0.59 | 1 | 1 | 0.65 | 0.00127 | 2 | 0 |
| Yes | FGFR2 | 7 | 7 | 0 | −0.81 | −0.57 | 91 | 77 | 0.90 | 0.00041 | 3 | 0 |
| Yes | FUBP1 | 2 | 2 | 0 | −0.70 | −0.58 | 1 | 1 | 0.75 | 5.51E−05 | 2 | 0 |
| Yes | H3F3A | 8 | 5 | 3 | −0.77 | 0.65 | 154 | 154 | 0.91 | 1.57E−07 | 2 | 2 |
| Yes | JAK1 | 2 | 1 | 1 | −0.62 | 0.64 | 1 | 1 | 0.75 | 0.00012 | 1 | 1 |
| Yes | KDM5C | 8 | 4 | 4 | −0.75 | 0.68 | 154 | 154 | 0.79 | 5.02E−05 | 2 | 1 |
| Yes | KMT2D | 10 | 0 | 10 | 0.56 | 0.76 | 246 | 245 | 0.82 | 0.00071 | 0 | 4 |
| Yes | MEN1 | 34 | 1 | 33 | −0.62 | 0.88 | 15092 | 9822 | 0.97 | 5.28E−05 | 0 | 4 |
| Yes | MLH1 | 4 | 4 | 0 | −0.65 | −0.55 | 11 | 11 | 0.69 | 0.0009 | 2 | 0 |
| Yes | MSH2 | 2 | 1 | 1 | −0.69 | 0.61 | 1 | 1 | 0.72 | 0.00018 | 1 | 1 |
| Yes | PBRM1 | 9 | 8 | 1 | −0.67 | 0.64 | 246 | 224 | 0.78 | 5.80E−05 | 2 | 1 |
| Yes | PRDM1 | 6 | 1 | 5 | −0.65 | 0.71 | 50 | 50 | 0.84 | 4.73E−06 | 1 | 2 |
| Yes | RNF43 | 4 | 4 | 0 | −0.83 | −0.58 | 4 | 3 | 0.90 | 8.97E−09 | 2 | 0 |
| Yes | SMAD2 | 24 | 24 | 0 | −0.83 | −0.56 | 10858 | 10858 | 0.98 | 2.10E−06 | 4 | 0 |
| Yes | SMO | 29 | 15 | 14 | −0.75 | 0.75 | 17875 | 17550 | 0.80 | 0.00027 | 2 | 2 |
| Yes | SOCS1 | 10 | 8 | 2 | −0.75 | 0.70 | 375 | 269 | 0.86 | 0.00108 | 4 | 0 |
| Yes | SOX9 | 9 | 0 | 9 | 0.55 | 0.66 | 246 | 246 | 0.73 | 0.00073 | 0 | 4 |
| Yes | SRSF2 | 10 | 9 | 1 | −0.67 | 0.60 | 375 | 291 | 0.90 | 0.00106 | 3 | 1 |
| Yes | TNFAIP3 | 18 | 10 | 8 | −0.72 | 0.71 | 4029 | 4029 | 0.90 | 1.41E−06 | 2 | 2 |
| Yes | TRAF7 | 14 | 0 | 14 | 0.55 | 0.84 | 1012 | 824 | 0.87 | 0.00025 | 0 | 4 |
| Yes | U2AF1 | 2 | 0 | 2 | 0.62 | 0.71 | 1 | 1 | 0.74 | 0.00013 | 0 | 2 |
| Yes | VHL | 8 | 8 | 0 | −0.77 | 0.60 | 154 | 153 | 0.92 | 0.00018 | 4 | 0 |
| Yes | AR | 1 | 1 | 0 | −0.64 | −0.64 | 0 | 0 | 0.00 | 0 | 0 | 0 |
| Yes | CARD11 | 1 | 0 | 1 | 0.63 | 0.63 | 0 | 0 | 0.00 | 0 | 0 | 0 |
| Yes | CASP8 | 1 | 0 | 1 | 0.62 | 0.62 | 0 | 0 | 0.00 | 0 | 0 | 0 |
| Yes | CDKN2C | 1 | 1 | 0 | −0.63 | −0.63 | 0 | 0 | 0.00 | 0 | 0 | 0 |
| Yes | MSH6 | 1 | 0 | 1 | 0.64 | 0.64 | 0 | 0 | 0.00 | 0 | 0 | 0 |
| No | AKT2 | 13 | 0 | 13 | 0.55 | 0.76 | 550 | 548 | 0.95 | 4.28E−08 | 0 | 4 |
| No | CD68 | 2 | 1 | 1 | −0.57 | 0.59 | 1 | 1 | 0.69 | 0.00042 | 1 | 1 |
| No | DSCAML1 | 5 | 1 | 4 | −0.56 | 0.66 | 25 | 25 | 0.84 | 0.0029 | 1 | 2 |
| No | FGF17 | 14 | 0 | 14 | 0.56 | 0.80 | 1079 | 1079 | 0.90 | 3.88E−05 | 0 | 4 |
| No | HK3 | 5 | 1 | 4 | −0.65 | 0.68 | 25 | 25 | 0.92 | 8.97E−05 | 1 | 3 |
| No | IFI30 | 4 | 1 | 3 | −0.55 | 0.68 | 11 | 4 | 0.70 | 0.00031 | 1 | 1 |
| No | RELB | 7 | 5 | 2 | −0.73 | 0.81 | 53 | 38 | 0.92 | 0.0001 | 0 | 2 |
| No | ZIC2 | 19 | 19 | 0 | −0.77 | −0.55 | 5016 | 5011 | 0.86 | 3.34E−05 | 4 | 0 |
| No | TOP1 | 1 | 0 | 1 | 0.58 | 0.58 | 0 | 0 | 0.00 | 0 | 0 | 0 |
| No | TRADD | 1 | 1 | 0 | −0.61 | −0.61 | 0 | 0 | 0.00 | 0 | 0 | 0 |
| Yes | CDKN2A | 17 | 17 | 0 | −0.82 | −0.60 | 3196 | 3196 | 0.89 | 1.00E−06 | 4 | 0 |
| Yes | EGFR | 22 | 0 | 22 | 0.56 | 0.77 | 9086 | 9055 | 0.86 | 1.73E−05 | 0 | 4 |
| Yes | EZH2 | 2 | 2 | 0 | −0.59 | −0.59 | 1 | 1 | 0.59 | 0.0236 | 2 | 0 |
| Yes | G011 | 4 | 1 | 3 | −0.59 | 0.67 | 11 | 11 | 0.81 | 0.01053 | 1 | 3 |
| Yes | GATA1 | 3 | 0 | 3 | 0.78 | 0.81 | 4 | 4 | 0.94 | 0.00027 | 0 | 2 |
| Yes | MYD88 | 16 | 16 | 0 | −0.75 | −0.56 | 2500 | 2391 | 0.85 | 0.00145 | 4 | 0 |
| Yes | RPL5 | 2 | 1 | 1 | −0.60 | 0.66 | 1 | 1 | 0.79 | 0.00287 | 1 | 1 |
| Yes | ALK | 0 | 0 | 0 | 0.00 | 0.00 | 0 | 0 | 0.00 | 0 | 0 | 0 |
| Yes | APC | 0 | 0 | 0 | 0.00 | 0.00 | 0 | 0 | 0.00 | 0 | 0 | 0 |
| Yes | ARID1A | 2 | 0 | 2 | 0.65 | 0.68 | 1 | 1 | 0.64 | 0.00138 | 0 | 2 |
| Yes | ARID1B | 0 | 0 | 0 | 0.00 | 0.00 | 0 | 0 | 0.00 | 0 | 0 | 0 |
| Yes | ARID2 | 0 | 0 | 0 | 0.00 | 0.00 | 0 | 0 | 0.00 | 0 | 0 | 0 |
| Yes | ASXL1 | 4 | 1 | 3 | −0.65 | 0.87 | 4 | 4 | 0.82 | 6.14E−06 | 1 | 1 |
| Yes | ATM | 0 | 0 | 0 | 0.00 | 0.00 | 0 | 0 | 0.00 | 0 | 0 | 0 |
| Yes | ATRX | 0 | 0 | 0 | 0.00 | 0.00 | 0 | 0 | 0.00 | 0 | 0 | 0 |
| Yes | AXIN1 | 18 | 1 | 17 | −0.76 | 0.87 | 2500 | 1818 | 0.79 | 0.00934 | 0 | 4 |
| Yes | B2M | 0 | 0 | 0 | 0.00 | 0.00 | 0 | 0 | 0.00 | 0 | 0 | 0 |
| Yes | BAP1 | 1 | 1 | 0 | −0.57 | −0.57 | 0 | 0 | 0.00 | 0 | 0 | 0 |
| Yes | BCL2 | 1 | 1 | 0 | −0.65 | −0.65 | 0 | 0 | 0.00 | 0 | 0 | 0 |
| Yes | BRAF | 0 | 0 | 0 | 0.00 | 0.00 | 0 | 0 | 0.00 | 0 | 0 | 0 |
| Yes | BRCA2 | 2 | 0 | 2 | 0.57 | 0.60 | 1 | 1 | 0.52 | 0.01977 | 0 | 2 |
| Yes | CBL | 0 | 0 | 0 | 0.00 | 0.00 | 0 | 0 | 0.00 | 0 | 0 | 0 |
| Yes | CDC73 | 0 | 0 | 0 | 0.00 | 0.00 | 0 | 0 | 0.00 | 0 | 0 | 0 |
| Yes | CDH1 | 2 | 1 | 1 | −0.73 | 0.85 | 0 | 0 | 0.00 | 0 | 0 | 0 |
| Yes | CEBPA | 0 | 0 | 0 | 0.00 | 0.00 | 0 | 0 | 0.00 | 0 | 0 | 0 |
| Yes | CIC | 7 | 0 | 7 | 0.55 | 0.85 | 50 | 50 | 0.70 | 0.01021 | 0 | 4 |
| Yes | CSF1R | 1 | 0 | 1 | 0.69 | 0.69 | 0 | 0 | 0.00 | 0 | 0 | 0 |
| Yes | CYLD | 0 | 0 | 0 | 0.00 | 0.00 | 0 | 0 | 0.00 | 0 | 0 | 0 |
| Yes | DNMT1 | 2 | 0 | 2 | 0.61 | 0.79 | 0 | 0 | 0.00 | 0 | 0 | 0 |
| Yes | EP300 | 2 | 1 | 1 | −0.66 | 0.61 | 1 | 1 | 0.64 | 0.0165 | 1 | 1 |
| Yes | ERBB2 | 11 | 10 | 1 | −0.90 | 0.67 | 309 | 207 | 0.87 | 0.00271 | 3 | 1 |
| Yes | FGFR3 | 13 | 5 | 8 | −0.70 | 0.90 | 781 | 751 | 0.89 | 6.74E−05 | 1 | 3 |
| Yes | FLT3 | 0 | 0 | 0 | 0.00 | 0.00 | 0 | 0 | 0.00 | 0 | 0 | 0 |
| Yes | FOXL2 | 0 | 0 | 0 | 0.00 | 0.00 | 0 | 0 | 0.00 | 0 | 0 | 0 |
| Yes | G0Q | 0 | 0 | 0 | 0.00 | 0.00 | 0 | 0 | 0.00 | 0 | 0 | 0 |
| Yes | G0S | 2 | 2 | 0 | −0.68 | −0.58 | 1 | 1 | 0.57 | 0.00591 | 2 | 0 |
| Yes | GATA2 | 0 | 0 | 0 | 0.00 | 0.00 | 0 | 0 | 0.00 | 0 | 0 | 0 |
| Yes | GATA3 | 0 | 0 | 0 | 0.00 | 0.00 | 0 | 0 | 0.00 | 0 | 0 | 0 |
| Yes | HNF1A | 1 | 1 | 0 | −0.57 | −0.57 | 0 | 0 | 0.00 | 0 | 0 | 0 |
| Yes | HRAS | 4 | 0 | 4 | 0.56 | 0.83 | 4 | 4 | 0.68 | 0.01264 | 0 | 2 |
| Yes | IDH1 | 0 | 0 | 0 | 0.00 | 0.00 | 0 | 0 | 0.00 | 0 | 0 | 0 |
| Yes | IDH2 | 2 | 0 | 2 | 0.56 | 0.87 | 0 | 0 | 0.00 | 0 | 0 | 0 |
| Yes | JAK2 | 0 | 0 | 0 | 0.00 | 0.00 | 0 | 0 | 0.00 | 0 | 0 | 0 |
| Yes | JAK3 | 0 | 0 | 0 | 0.00 | 0.00 | 0 | 0 | 0.00 | 0 | 0 | 0 |
| Yes | KDM6A | 0 | 0 | 0 | 0.00 | 0.00 | 0 | 0 | 0.00 | 0 | 0 | 0 |
| Yes | KIT | 0 | 0 | 0 | 0.00 | 0.00 | 0 | 0 | 0.00 | 0 | 0 | 0 |
| Yes | KLF4 | 11 | 10 | 1 | −0.81 | 0.73 | 550 | 550 | 0.78 | 0.00018 | 3 | 1 |
| Yes | KMT2C | 0 | 0 | 0 | 0.00 | 0.00 | 0 | 0 | 0.00 | 0 | 0 | 0 |
| Yes | KRAS | 0 | 0 | 0 | 0.00 | 0.00 | 0 | 0 | 0.00 | 0 | 0 | 0 |
| Yes | MAP2K1 | 0 | 0 | 0 | 0.00 | 0.00 | 0 | 0 | 0.00 | 0 | 0 | 0 |
| Yes | MAP3K1 | 0 | 0 | 0 | 0.00 | 0.00 | 0 | 0 | 0.00 | 0 | 0 | 0 |
| Yes | MED12 | 0 | 0 | 0 | 0.00 | 0.00 | 0 | 0 | 0.00 | 0 | 0 | 0 |
| Yes | MET | 1 | 1 | 0 | 0.74 | −0.74 | 0 | 0 | 0.00 | 0 | 0 | 0 |
| Yes | MPL | 0 | 0 | 0 | 0.00 | 0.00 | 0 | 0 | 0.00 | 0 | 0 | 0 |
| Yes | NCOR1 | 0 | 0 | 0 | 0.00 | 0.00 | 0 | 0 | 0.00 | 0 | 0 | 0 |
| Yes | NF1 | 0 | 0 | 0 | 0.00 | 0.00 | 0 | 0 | 0.00 | 0 | 0 | 0 |
| Yes | NF2 | 1 | 0 | 1 | 0.63 | 0.63 | 0 | 0 | 0.00 | 0 | 0 | 0 |
| Yes | NFE2L2 | 0 | 0 | 0 | 0.00 | 0.00 | 0 | 0 | 0.00 | 0 | 0 | 0 |
| Yes | NOTCH1 | 8 | 1 | 7 | −0.71 | 0.88 | 50 | 50 | 0.86 | 4.03E−05 | 0 | 4 |
| Yes | NOTCH2 | 0 | 0 | 0 | 0.00 | 0.00 | 0 | 0 | 0.00 | 0 | 0 | 0 |
| Yes | NPM1 | 0 | 0 | 0 | 0.00 | 0.00 | 0 | 0 | 0.00 | 0 | 0 | 0 |
| Yes | NRAS | 1 | 1 | 0 | −0.58 | −0.58 | 0 | 0 | 0.00 | 0 | 0 | 0 |
| Yes | PAX5 | 0 | 0 | 0 | 0.00 | 0.00 | 0 | 0 | 0.00 | 0 | 0 | 0 |
| Yes | PDGFRA | 8 | 8 | 0 | −0.82 | −0.58 | 154 | 154 | 0.80 | 0.00022 | 4 | 0 |
| Yes | PHF6 | 0 | 0 | 0 | 0.00 | 0.00 | 0 | 0 | 0.00 | 0 | 0 | 0 |
| Yes | PIK3CA | 1 | 0 | 1 | 0.65 | 0.65 | 0 | 0 | 0.00 | 0 | 0 | 0 |
| Yes | PIK3R1 | 0 | 0 | 0 | 0.00 | 0.00 | 0 | 0 | 0.00 | 0 | 0 | 0 |
| Yes | PPP2R1A | 0 | 0 | 0 | 0.00 | 0.00 | 0 | 0 | 0.00 | 0 | 0 | 0 |
| Yes | PTCH1 | 1 | 1 | 0 | −0.69 | −0.69 | 0 | 0 | 0.00 | 0 | 0 | 0 |
| Yes | PTEN | 2 | 0 | 2 | 0.61 | 0.67 | 1 | 1 | 0.64 | 0.00356 | 0 | 2 |
| Yes | PTPN11 | 0 | 0 | 0 | 0.00 | 0.00 | 0 | 0 | 0.00 | 0 | 0 | 0 |
| Yes | RB1 | 0 | 0 | 0 | 0.00 | 0.00 | 0 | 0 | 0.00 | 0 | 0 | 0 |
| Yes | RET | 1 | 1 | 0 | −0.72 | −0.72 | 0 | 0 | 0.00 | 0 | 0 | 0 |
| Yes | RUNX1 | 0 | 0 | 0 | 0.00 | 0.00 | 0 | 0 | 0.00 | 0 | 0 | 0 |
| Yes | SETBP1 | 0 | 0 | 0 | 0.00 | 0.00 | 0 | 0 | 0.00 | 0 | 0 | 0 |
| Yes | SETD2 | 1 | 0 | 1 | 0.73 | 0.73 | 0 | 0 | 0.00 | 0 | 0 | 0 |
| Yes | SF3B1 | 0 | 0 | 0 | 0.00 | 0.00 | 0 | 0 | 0.00 | 0 | 0 | 0 |
| Yes | SMAD4 | 3 | 0 | 3 | 0.61 | 0.75 | 4 | 4 | 0.63 | 0.00186 | 0 | 2 |
| Yes | SMARCA4 | 2 | 0 | 2 | 0.66 | 0.76 | 0 | 0 | 0.00 | 0 | 0 | 0 |
| Yes | SMARCB1 | 5 | 0 | 5 | 0.57 | 0.83 | 1 | 1 | 0.65 | 0.00666 | 0 | 2 |
| Yes | SPOP | 3 | 3 | 0 | −0.69 | −0.59 | 4 | 4 | 0.66 | 0.00089 | 2 | 0 |
| Yes | STAG2 | 0 | 0 | 0 | 0.00 | 0.00 | 0 | 0 | 0.00 | 0 | 0 | 0 |
| Yes | STK11 | 41 | 2 | 39 | −0.88 | 0.76 | 1925 | 1925 | 0.81 | 4.55E−05 | 0 | 4 |
| Yes | TET2 | 0 | 0 | 0 | 0.00 | 0.00 | 0 | 0 | 0.00 | 0 | 0 | 0 |
| Yes | TP53 | 0 | 0 | 0 | 0.00 | 0.00 | 0 | 0 | 0.00 | 0 | 0 | 0 |
| Yes | TSC1 | 4 | 1 | 3 | −0.67 | 0.95 | 4 | 4 | 0.78 | 0.0085 | 1 | 2 |
| Yes | TSHR | 0 | 0 | 0 | 0.00 | 0.00 | 0 | 0 | 0.00 | 0 | 0 | 0 |
| Yes | WT1 | 0 | 0 | 0 | 0.00 | 0.00 | 0 | 0 | 0.00 | 0 | 0 | 0 |
| No | CHI3L1 | 19 | 18 | 1 | −0.75 | 0.58 | 4983 | 4976 | 0.96 | 0.00017 | 3 | 1 |
| No | DLL3 | 7 | 1 | 6 | −0.65 | 0.76 | 91 | 91 | 0.82 | 3.45E−05 | 1 | 3 |
| No | EN1 | 38 | 38 | 0 | −0.73 | −0.55 | 59500 | 58737 | 0.85 | 6.85E−05 | 4 | 0 |
| No | GDF15 | 68 | 65 | 3 | −0.80 | 0.78 | 92131 | 46116 | 0.90 | 8.11E−06 | 4 | 0 |
| No | IGFBP6 | 6 | 4 | 2 | −0.67 | 0.63 | 50 | 49 | 0.87 | 1.25E−07 | 1 | 1 |
| No | MBP | 23 | 23 | 0 | −0.75 | −0.56 | 10879 | 10879 | 0.85 | 7.89E−06 | 4 | 0 |
| No | NES | 14 | 13 | 1 | −0.76 | 0.62 | 1079 | 1035 | 0.84 | 0.00041 | 4 | 0 |
| No | OLIG2 | 11 | 7 | 4 | −0.77 | 0.82 | 550 | 550 | 0.90 | 1.92E−07 | 2 | 2 |
| No | PDGFA | 35 | 31 | 4 | −0.72 | 0.69 | 41416 | 39485 | 0.91 | 7.58E−07 | 4 | 0 |
| No | SOX10 | 34 | 33 | 1 | −0.76 | 0.61 | 20826 | 20826 | 0.92 | 3.07E−06 | 4 | 0 |
| No | VIPR2 | 23 | 17 | 6 | −0.72 | 0.70 | 10879 | 9544 | 0.85 | 0.00495 | 3 | 1 |
| No | ACSS3 | 1 | 0 | 1 | 0.73 | 0.73 | 0 | 0 | 0.00 | 0 | 0 | 0 |
| No | AQP9 | 1 | 1 | 0 | −0.69 | −0.69 | 0 | 0 | 0.00 | 0 | 0 | 0 |
| No | ASXL3 | 0 | 0 | 0 | 0.00 | 0.00 | 0 | 0 | 0.00 | 0 | 0 | 0 |
| No | BATF | 0 | 0 | 0 | 0.00 | 0.00 | 0 | 0 | 0.00 | 0 | 0 | 0 |
| No | BCAT1 | 0 | 0 | 0 | 0.00 | 0.00 | 0 | 0 | 0.00 | 0 | 0 | 0 |
| No | CA12 | 12 | 5 | 7 | −0.74 | 0.63 | 781 | 779 | 0.72 | 0.00119 | 2 | 2 |
| No | CASP5 | 0 | 0 | 0 | 0.00 | 0.00 | 0 | 0 | 0.00 | 0 | 0 | 0 |
| No | CD163 | 0 | 0 | 0 | 0.00 | 0.00 | 0 | 0 | 0.00 | 0 | 0 | 0 |
| No | CD177 | 0 | 0 | 0 | 0.00 | 0.00 | 0 | 0 | 0.00 | 0 | 0 | 0 |
| No | DMRTA2 | 0 | 0 | 0 | 0.00 | 0.00 | 0 | 0 | 0.00 | 0 | 0 | 0 |
| No | ERBB3 | 2 | 2 | 0 | −0.77 | −0.57 | 1 | 1 | 0.63 | 0.01542 | 2 | 0 |
| No | FBXO3 | 0 | 0 | 0 | 0.00 | 0.00 | 0 | 0 | 0.00 | 0 | 0 | 0 |
| No | FCGR2B | 3 | 2 | 1 | −0.74 | 0.62 | 4 | 4 | 0.68 | 0.00655 | 2 | 0 |
| No | FGF9 | 0 | 0 | 0 | 0.00 | 0.00 | 0 | 0 | 0.00 | 0 | 0 | 0 |
| No | FPR2 | 0 | 0 | 0 | 0.00 | 0.00 | 0 | 0 | 0.00 | 0 | 0 | 0 |
| No | GABRB2 | 0 | 0 | 0 | 0.00 | 0.00 | 0 | 0 | 0.00 | 0 | 0 | 0 |
| No | GLYATL2 | 0 | 0 | 0 | 0.00 | 0.00 | 0 | 0 | 0.00 | 0 | 0 | 0 |
| No | GRIA4 | 0 | 0 | 0 | 0.00 | 0.00 | 0 | 0 | 0.00 | 0 | 0 | 0 |
| No | GRID2 | 0 | 0 | 0 | 0.00 | 0.00 | 0 | 0 | 0.00 | 0 | 0 | 0 |
| No | LGI3 | 0 | 0 | 0 | 0.00 | 0.00 | 0 | 0 | 0.00 | 0 | 0 | 0 |
| No | LIF | 1 | 1 | 0 | −0.62 | −0.62 | 0 | 0 | 0.00 | 0 | 0 | 0 |
| No | LILRB2 | 0 | 0 | 0 | 0.00 | 0.00 | 0 | 0 | 0.00 | 0 | 0 | 0 |
| No | LYVE1 | 0 | 0 | 0 | 0.00 | 0.00 | 0 | 0 | 0.00 | 0 | 0 | 0 |
| No | SGCD | 3 | 3 | 0 | −0.61 | −0.55 | 4 | 4 | 0.58 | 0.00545 | 2 | 0 |
| No | SLC17A7 | 0 | 0 | 0 | 0.00 | 0.00 | 0 | 0 | 0.00 | 0 | 0 | 0 |
| No | SNCG | 1 | 0 | 1 | 0.59 | 0.59 | 0 | 0 | 0.00 | 0 | 0 | 0 |
| No | SOX2 | 0 | 0 | 0 | 0.00 | 0.00 | 0 | 0 | 0.00 | 0 | 0 | 0 |
| No | SPHK1 | 1 | 0 | 1 | 0.59 | 0.59 | 0 | 0 | 0.00 | 0 | 0 | 0 |
| No | TLR2 | 0 | 0 | 0 | 0.00 | 0.00 | 0 | 0 | 0.00 | 0 | 0 | 0 |
| No | TLR4 | 0 | 0 | 0 | 0.00 | 0.00 | 0 | 0 | 0.00 | 0 | 0 | 0 |
| No | ZNF676 | 1 | 0 | 1 | 0.58 | 0.58 | 0 | 0 | 0.00 | 0 | 0 | 0 |

### Example 7: Gene-Regulatory Units Compose Cis-Regulatory Networks

Next, the relationships between gene-regulatory units of given genes were analyzed. Clearly, silencer and enhancer units of the same gene tend to be reversely coordinated across the tumors, so tumors with unmethylated silencers and methylated enhancers display lower expression of the gene, whereas tumors with higher expression of the gene have the opposite arrangements (FIG. 3D-E, 13A-B). Hence, enhancers and silencers of a given gene may be spread over large portions of the gene domain, and yet maintain coordinated levels of activities. These networks of cooperating enhancers and silencers are termed the cis-regulatory network of genes.

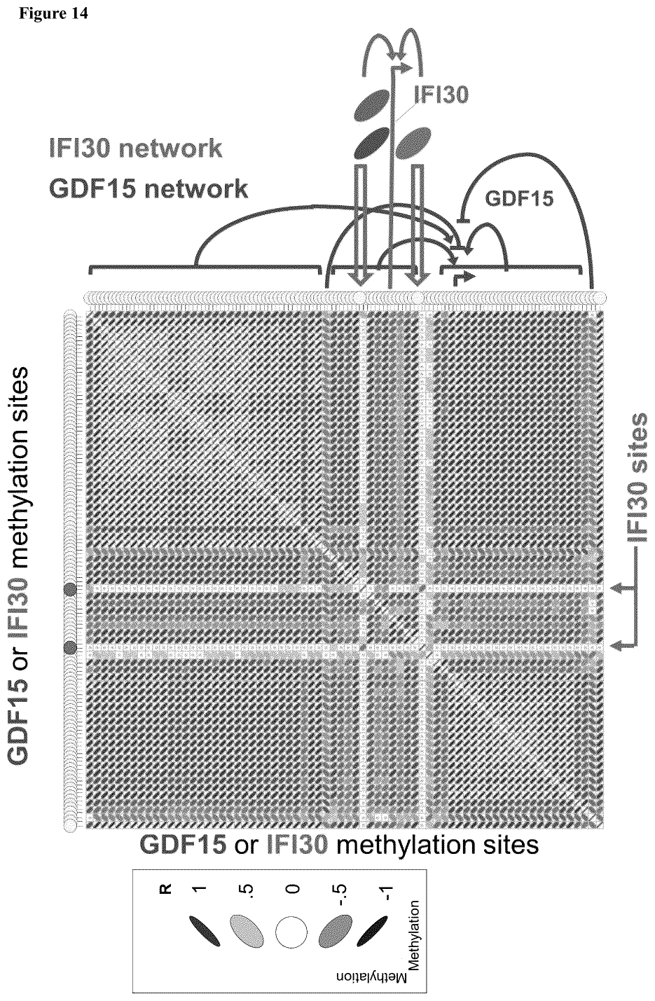

It was previously unclear how different genes within the same regulatory domain maintained independent regulatory profiles. To gain understanding of the issue the relationships between networks of neighboring genes were analyzed. Interestingly, it was found that units of particular genes, even if intermixed with units of other genes, maintain their own inter-network coordination, whereas units of different genes, even when close together, display independent activities (FIG. 14). These structures of spatially intermixed, gene-specific networks allow independent regulation of genes within shared regulatory domains.

### Example 8: Mathematical Modulation Signifies Key Network Sites

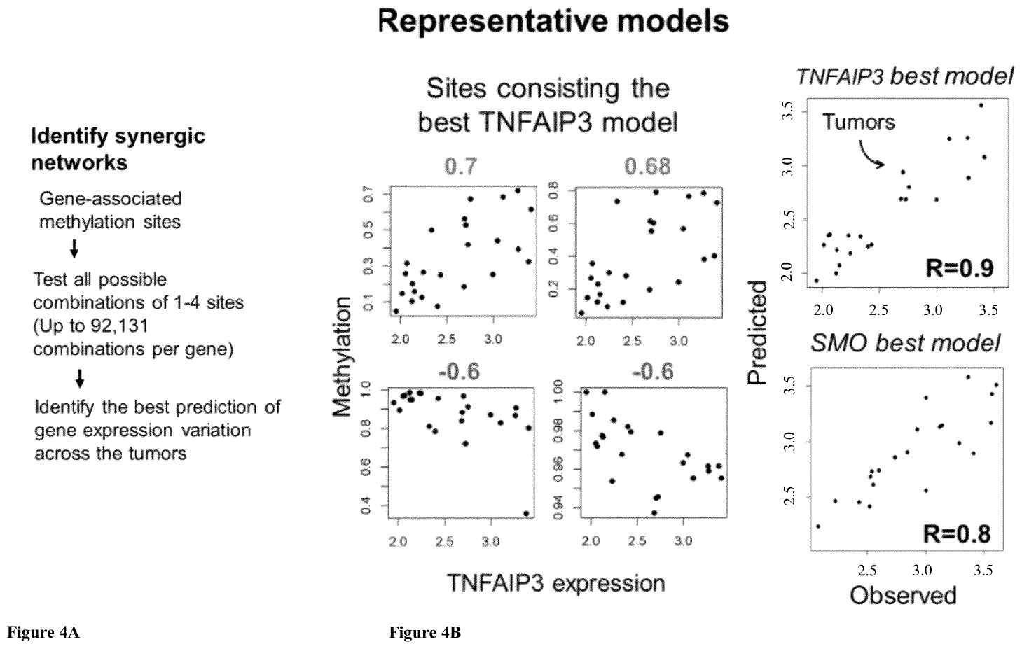

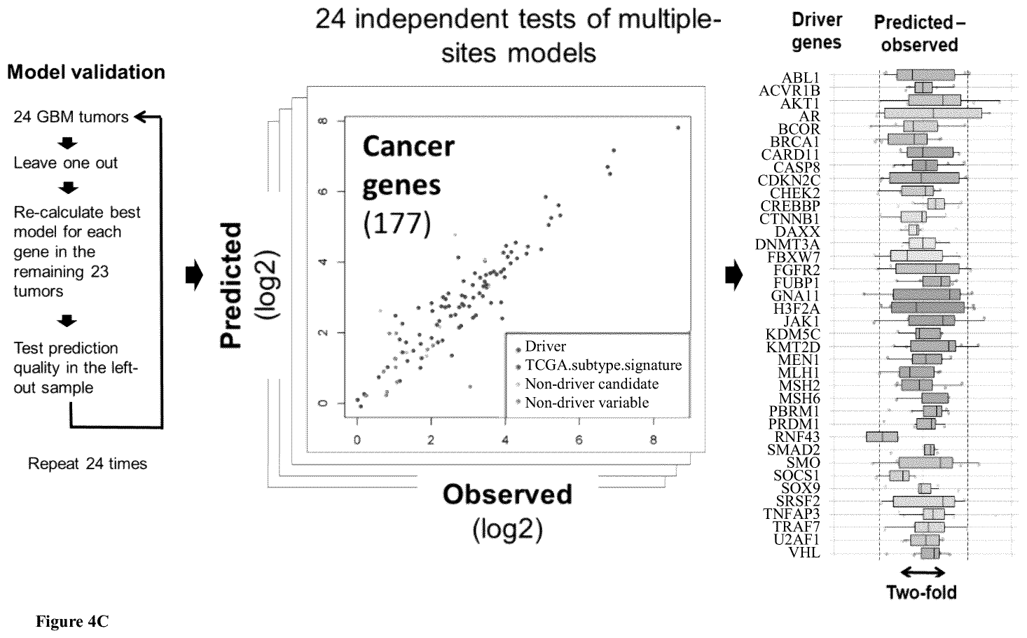

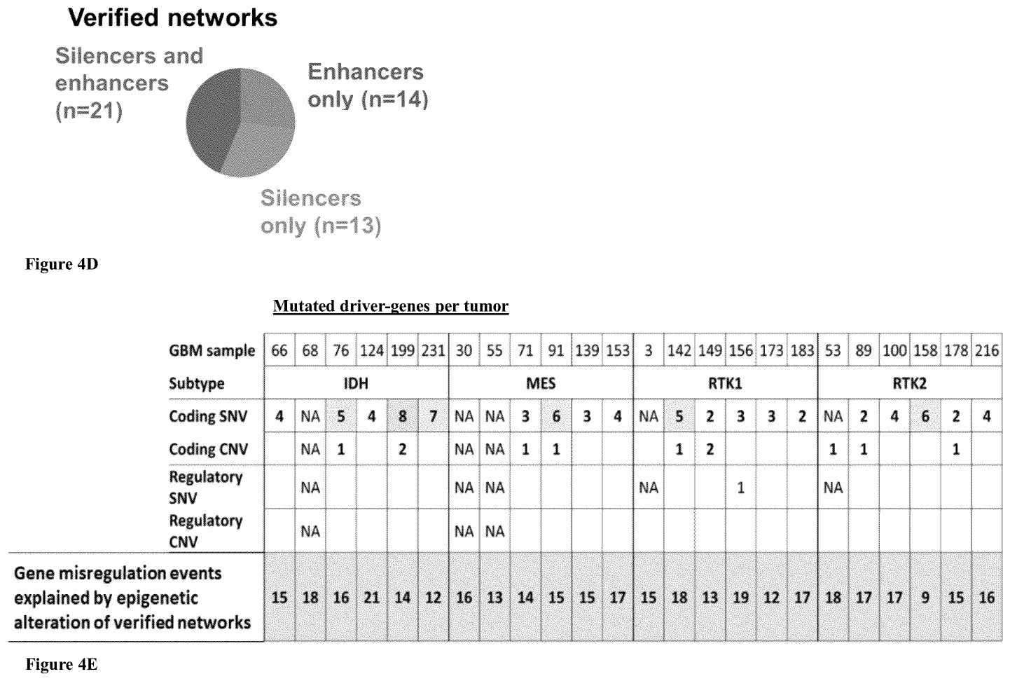

The interaction between networked silencers and enhancers was further explored by examining multiplexed effects on gene expression: Given a certain effect of an arbitrarily selected regulatory site on expression of a controlled gene, it was asked whether multiplexed models that consider additional associated sites provide improved expression prediction. Therefore, redundant regulatory sites should provide no improvement, whereas antagonists or synergistic sites are expected to improve the prediction provided by each of the sites alone. Using stepwise analyses, the best models of possible combinations of up to four sites were identified (FIG. 4A). For example, the eighteen TNFAIP3-associated sites produced predictive R-values ranging between −0.72 and 0.71 for each individual site (Table 4). The tests of the 4,029 possible combinations of one to four sites out of the 18 cis-regulatory circuits, revealed a model that incorporated the methylation levels of two positive and two negative sites, providing better prediction power than each of the sites alone (R=0.9, p=1.41E-06). Hence, the revealed model signifies the methylation sites that provide the best description of the gene expression-variation. By that, it hints to the particular regulatory sites, out of all associated sites, which are most significant to the regulation of the gene. Similarly, the best model for the SMO gene, incorporating the methylation level of two positive and two negative sites, provided better prediction power (R=0.8, p=0.00027) than each of the 29 associated sites alone. As in the case of TNFAIP3, these sites resided within positive and negative regulatory units (FIG. 4B). Note that the model used no preliminary assumptions regarding the nature of the most predictive sites. Therefore, the fact that both positive and negative sites were used by the produced models, suggests that they are jointly responsible to the determination of gene expression level.

Overall, out of 105 genes with significant models, the expression of 58 genes were best predicted by synergic combinations of sites, providing better prediction than each of the sites alone (Table 5). The power of mathematically-significant models was further verified by testing their predictions in tumors that were not used during the model development (FIG. 4C, 15). Of the 48 genes with validated synergic or single-site models, silencers were involved in the regulation of 34 genes (FIG. 4D).

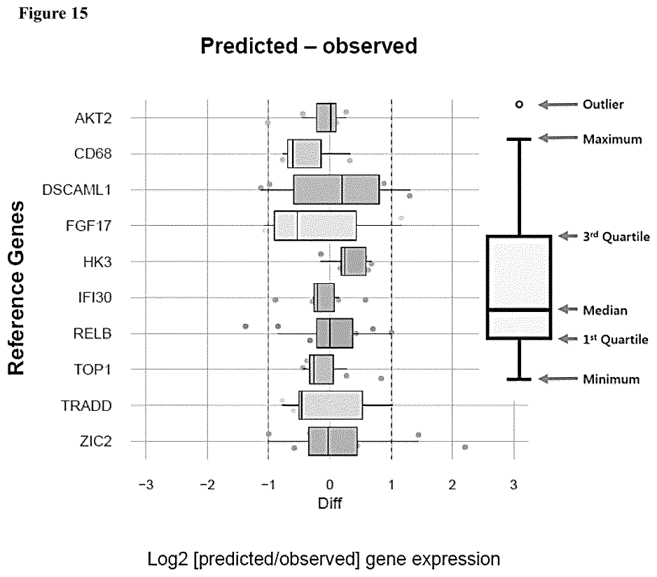

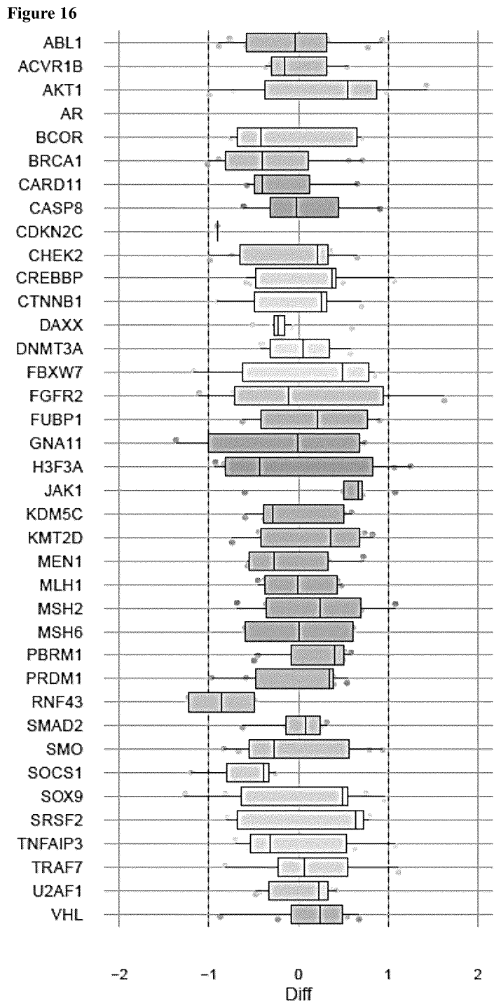

To eliminate possible bias due to the limit of up to four associated sites in the gene-expression models, the models were rebuilt using a different approach in which no limitation on the number of participating sites was applied. This independent analysis yielded very similar results (FIG. 16), with an average of 3.8 contributing sites per gene-model across all genes, thus indicating the robustness of the model-development method.

It was concluded that mathematical modulation of methylation effects provides an efficient way to identify contributing regulatory sites and to explore the organization and function of gene-specific networks. Out of the many gene-associated sites presented in gene regulatory domains, and numerus possible combinations of the associated sites, this approach efficiently identified guiding cis-regulatory sites and networks.

### Example 9: Epigenetically-Retuned Cis-Regulatory Networks Guide Gene Transformation

Finally, the contributions of mutations in silencers, enhancers, or coding sequences to driver gene malfunction were compared. In the majority (68.4%) of the tumors, fewer than five driver genes were affected by nonsynonymous or copy number mutations (FIG. 4E), in line with previous analyses of this cancer. To reveal the effect of regulatory sequence mutations, the uncovered silencers and enhancers in eight of the patients were deep-sequenced, and the effect of sequence variations on expression of the associated genes was analyzed. Notably, only one possible event was revealed, aside from common sequence polymorphisms. As current models of cancer predict a minimum number of five to eight mutated driver genes, regulatory and coding sequence mutations alone cannot explain the appearance of a majority of the GBM tumors. In contrast, all tumors included more than eight abnormally expressed driver genes that associated with methylation-tuned regulatory units and were explained by confirmed methylation-based models of expression variations (FIG. 4E). Silencers were involved, alone or in cooperation with enhancers, in almost two-thirds of these mis-regulation events (Table 6) and were implicated in the malfunction of genes driving a wide range of cancer initiation and progression processes (FIG. 17). It was concluded that epigenetic retuning of networked regulatory elements plays a prime role in the malfunction of cancer driver-genes.

TABLE 6
Genes affected by regulatory or coding mutation.

| | | Fraction of | Fraction of | | |
|---|---|---|---|---|---|
| | | tumors with | tumors with | | |
| Mu- | | coding | abnormal | Expression | |
| tation | Driver | mutations | expression ^{(a)} | variation | Silencer |
| type | gene | (%) | (%) | explained ^{(b)} | involved |
| Reg- | SMO | 0 | 95.8 | Yes | Yes |
| ulatory | SOX9 | 0 | 79.2 | Yes | Yes |
| | CASP8 | 0 | 70.8 | Yes | Yes |
| | TNFAIP3 | 0 | 70.8 | Yes | Yes |
| | H3F3A | 0 | 54.2 | Yes | Yes |
| | ABL1 | 0 | 45.8 | Yes | Yes |
| | DAXX | 0 | 29.2 | Yes | Yes |
| | MSH6 | 0 | 29.2 | Yes | Yes |
| | JAK1 | 0 | 8.3 | Yes | Yes |
| | U2AF1 | 0 | 8.3 | Yes | Yes |
| | SOCS1 | 0 | 4.2 | Yes | Yes |
| | SRSF2 | 0 | 4.2 | Yes | Yes |
| | FBXW7 | 0 | 100 | Yes | No |
| | FGFR2 | 0 | 79.2 | Yes | No |
| | AR | 0 | 70.8 | Yes | No |
| | ZIC2 | 0 | 12.5 | Yes | No |
| | CHEK2 | 0 | 66.7 | Yes | No |
| | CTNNB1 | 0 | 8.3 | Yes | No |
| | MLH1 | 0 | 8.3 | Yes | No |
| | SMAD2 | 0 | 4.2 | Yes | No |
| | VHL | 0 | 4.2 | Yes | No |
| Reg- | BRCA1 | 21.1 | 83.3 | Yes | Yes |
| ulatory | TRAF7 | 5.3 | 41.7 | Yes | Yes |
| and | AKT1 | 5.3 | 20.8 | Yes | Yes |
| coding | PRDM1 | 10.5 | 0.8 | Yes | Yes |
| | PBRM1 | 5.3 | 12.5 | Yes | Yes |
| | MSH2 | 10.5 | 8.3 | Yes | Yes |
| | MEN1 | 5.3 | 4.2 | Yes | Yes |
| | CREBBP | 10.5 | 4.2 | Yes | Yes |
| | CDKN2C | 5.3 | 100 | Yes | No |
| | FUBP1 | 5.3 | 8.3 | Yes | No |
| Coding | TP53 | 47 | 100 | No | — |

^{(a)} Two-fold or more expression differences from normal brain samples.
^{(b)} By verified methylation-based models of expression variation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for determining a driver gene of a pathological condition in a subject in need thereof, the method comprising:
a. receiving measurements of DNA methylation within a plurality of non-promoter cis-regulatory sequences from said subject;
b. determining from said received measurements a total regulatory effect of non-promoter cis-regulatory sequences upon at least one potential driver gene of said pathological condition; and
c. selecting said at least one potential driver gene as a driver of said pathological condition in said subject when said total regulatory effect is beyond a predetermined threshold;
thereby determining a driver of a pathological condition in a subject.

2. The method of claim 1, wherein said measurements of DNA methylation are obtained by:
a. obtaining DNA from a biological sample from said subject;
b. isolating a plurality of cis-regulatory sequences from said obtained DNA; and
c. measuring DNA methylation within said plurality of isolated cis-regulatory sequences.

3. The method of claim 2, wherein at least one of:
a. said measuring DNA methylation comprises bisulfite sequencing of said plurality of isolated sequences;
b. said biological sample is selected from: tissue, blood, lymph, cerebral spinal fluid, urine, breast milk, feces, saliva, tumor tissue and tumor fluid;
c. said biological sample is a tumor biopsy; and
d. said isolating comprises binding probes to said cis-regulatory sequences and isolating said hybridized probes.

4. (canceled)

5. (canceled)

6. (canceled)

7. (canceled)

8. The method of claim 73, wherein said isolating comprises binding probes to said cis-regulatory sequences and isolating said hybridized probes and said probes binds histone 3 lysine 4 monomethylated (H3K4me1) chromatin.

9. The method of claim 37, wherein said isolating comprises binding probes to said cis-regulatory sequences and isolating said hybridized probes and said probe is a nucleic acid probe that hybridizes to said cis-regulatory sequence and comprises a non-nucleic acid capture moiety and wherein said isolating comprises capturing said capture moiety to a capturing molecule.

10. (canceled)

11. The method of claim 9 or 10, wherein said nucleic acid probe comprises a sequence selected from SEQ ID NO: 28-38077.

12. The method of claim 1, wherein
a. said plurality of non-promoter cis-regulatory sequences are located within 1 megabase upstream or downstream of a transcriptional start site of said at least one potential driver gene;
b. the regulatory effect of each cis-regulatory sequence is determined independently or is determined in combination with at least one other cis-regulatory sequence;
c. at least one measured cis-regulatory sequence comprises more than one CpG dinucleotide and wherein a measurement from at least one of said more than one CpG dinucleotides within said cis-regulatory sequence is received;
d. a regulatory effect of each non-promoter cis-regulatory sequence is determined separately and summed to produce said total regulatory effect, or wherein total regulatory effect for at least two non-promoter cis-regulatory sequences is determined simultaneously;
e. said non-promoter cis-regulatory sequences are selected from sequences located between genomic positions provided in Table 4; or
f. measurements of DNA methylation within non-promoter cis-regulatory sequences of a panel of potential driver genes are received.

13. The method of claim 1, wherein said plurality of non-promoter cis-regulatory sequences are selected from enhancer and repressor elements, comprise at least one repressor element, comprise at least 4 distinct cis-regulatory sequences or a combination thereof.

14. (canceled)

15. (canceled)

16. (canceled)

17. (canceled)

18. The method of claim 1, wherein said determining comprises at least one of:
a. testing each of said plurality of non-promoter cis-regulatory sequences in an expression assay, wherein said assay measures the regulatory effect of a non-promoter cis-regulatory sequence on expression of a coding sequence and wherein said testing comprises testing methylated and unmethylated copies of each of said plurality of non-promoter cis-regulatory sequences;
b. comparing said received measurements to a database comprising potential driver genes, methylation status of non-promoter cis-regulatory sequences of said database genes, and regulatory effects of said non-promoter cis regulatory sequences on said database genes; and
c. applying a machine learning algorithm to said received measurements, wherein said machine learning algorithm has been trained on non-promoter cis-regulatory sequences with known methylation status and known regulatory effect on a driver gene.

19. (canceled)

20. The method of claim 18 or 19, wherein said determining comprises applying a machine learning model and wherein said machine learning algorithm has been trained on:
a. single non-promoter cis-regulatory sequences;
b. genes and at least one of each gene's non-promoter cis-regulatory sequences;
c. genes and a plurality of each gene's non-promoter cis-regulatory sequences; or
d. genes and all of each gene's non-promoter cis-regulatory sequences.

21. The method of claim 1, wherein said predetermined threshold is derived from a predetermined standard regulatory effect for said non-promoter cis-regulatory sequences of said at least one potential driver gene, and wherein said predetermined standard regulatory effect is determined in any one of:
a. cells grown in culture;
b. cells from a healthy subject; and
c. cells from a subject suffering from a pathological condition.

22. (canceled)

23. The method of claim 1, further comprising confirming aberrant expression of said selected driver gene in a sample from said subject.

24. The method of claim 1, wherein said pathological condition is cancer.

25. The method of claim 24, wherein said cancer is glioblastoma.

26. The method of claim 24, wherein a potential driver gene is any one of the driver genes provided in Table 3 or any of the genes provided in Table 6 or wherein total regulatory effect on a panel of driver genes are determined, and said panel is selected from the genes provided in Table 6.

27. (canceled)

28. (canceled)

29. The method of claim 1, for diagnosing a pathological condition or increased risk of developing a pathological condition.

30. The method of claim 1, further comprising administering a medicament that targets said driver, DNA methylation, or DNA methylation machinery.

31. A kit, comprising nucleotide probes that hybridize to non-promoter cis-regulatory sequences of a plurality of genes selected from genes provided in Table 3, Table 4 or Table 6.

32. The kit of claim 31, wherein at least one of:
a. said plurality of genes is selected from the genes provided in Table 6;
b. said non-promoter cis-regulatory sequences are located between genomic positions provided in Table 4; and
c, wherein said probes are selected from SEQ ID NO: 28-38077.

33. (canceled)

34. (canceled)

35. (canceled)

36. A computer program product for determining a driver gene for a pathological condition, comprising a non-transitory computer-readable storage medium having program code embodied thereon, the program code executable by at least one hardware processor to:
a. receive measurements of DNA methylation within a plurality of non-promoter cis-regulatory sequences;
b. determine from said received measurements a total regulatory effect of non-promoter cis-regulatory sequences upon at least one potential driver gene of said pathological condition; and
c. select said at least one potential driver gene as a driver of said pathological condition when said total regulatory effect is beyond a predetermined threshold.
